SEQUENCE LISTING (118 sequences)

SEQ ID NO: 1 - protein, 305 aa, Streptococcus agalactiae
MGQKSKISLATNIRIWIFRLIFLAGFLVLAFPIVSQVMYFQASHANINAFKEAVTKIDRVEINRRLELAYAYNASIAGAKTNGEYPALKDPYSAEQKQAGVVEYARMLEVKEQIGHVIIPRINQDIPIYAGSAEENLQRGVGHLEGTSLPVGGESTHAVLTAHRGLPTAKLFTNLDKVTVGDRFYIEHIGGKIAYQVDQIKVIAPDQLEDLYVIQGEDHVTLLTCTPYMINSHRLLVRGKRIPYVEKTVQKDSKTFRQQQYLTYAMWVVVGLILLSLLIWFKKTKQKKRRKNEKAASQNSHNNSK

SEQ ID NO: 2 - protein, 283 aa, Streptococcus agalactiae
  GBS Sortase C2 of PI-1
MKKRLVKIVTIIRNNKIRTLIFVMGSLILLFPIVSQVSYYLASHQNINQFKREVAKIDTNTVERRIALANAYNETLSRNPLLIDPFTSKQKEGLREYARMLEVHEQIGHVAIPSIGVDIPIYAGTSETVLQKGSGHLEGTSLPVGGLSTHSVLTAHRGLPTARLFTDLNKVKKGQIFYVTNIKETLAYKVVSIKVVDPTALSEVKIVNGKDYITLLTCTPYMINSHRLLVKGERIPYDSTEAEKHKEQTVQDYRLSLVLKILLVLLIGLFIVIMMRRWMQHRQ

SEQ ID NO: 3 - protein, 292 aa, Streptococcus agalactiae
  GBS Sortas C1 of PI-2a
MKTKKIIKKTKKKKSNLPFIILFLIGLSILLYPVVSRFYYTIESNNQTQDFERAAKKLSQKEINRRMALAQAYNDSLNNVHLEDPYEKKRIQKGIAEYARMLEVSEKIGIISVPKIGQKLPIFAGSSQEVLSKGAGHLEGTSLPIGGNSTHTVITAHSGIPDKELFSNLKKLKKGDKFYIQNIKETIAYQVDQIKVVTPDNFSDLLVVPGHDYATLLTCTPIMVNTHRLLVRGHRIPYKGPIDEKLIKDGHLNTIYRYLFYISLVIIAWLLWLIKRQRQKNRLSSVRKGIES

SEQ ID NO: 4 - protein, 294 aa, Streptococcus agalactiae
  GBS Sortase C2 of PI-2a
MRGKFQKNLKKSVVLNRWMNIGLILLFLVGLLITSYPFISNWYYNIKANNQVTNFDNQTQKLNAKEINRRFELAKAYNRTLDPSRLSDPYTEKEKKGIAEYAHMLEITEMIGYIDIPSIKQKLPIYAGTTSSVLEKGSGHLEGTSLPIGGKSSHTVITAHRGLPKAKLFTDLDKLKKGKIFYIHNIKEVLAYKVDQISVVKPDNFSKLLVVKGKDYATLLTCTPYSINSHRLLVRGHRIKYVPPVKEKNYLMKELQTHYKLYFLLSILVILILVALLLYLKRKFKERKRKGNQK

SEQ ID NO: 5 - protein, 268 aa, Streptococcus agalactiae
  GBS Sortase C1 of PI-2b
MAYPSLANYWNSFHQSRAIMDYQDRVTHMDENDYKKIINRAKEYNKQFKTSGMKWHMTSQERLDYNSQLAIDKTGNMGYISIPKINIKLPLYHGTSEKVLQTSIGHLEGSSLPIGGDSTHSILSGHRGLPSSRLFSDLDKLKVGDHWTVSILNETYTYQVDQIRTVKPDDLRDLQIVKGKDYQTLVTCTPYGVNTHRLLVRGHRVPNDNGNALVVAEAIQIEPIYIAPFIAIFLTLILLLISLEVTRRARQRKKILKQAMRKEENNDL

SEQ ID NO: 6 - protein, 279 aa, Streptococcus pneumoniae
  Pneumococcus Sortase C1
MAVMAYPLVSRLYYRVESNQQIADFDKEKATLDEADIDERMKLAQAFNDSLNNVVSGDPWSEEMKKKGRAEYARMLEIHERMGHVEIPVIDVDLPVYAGTAEEVLQQGAGHLEGTSLPIGGNSTHAVITAHTGLPTAKMFTDLTKLKVGDKFYVHNIKEVMAYQVDQVKVIEPTNFDDLLIVPGHDYVTLLTCTPYMINTHRLLVRGHRIPYVAEVEEEFIAANKLSHLYRYLFYVAVGLIVILLWIIRRLRKKKKQPEKALKALKAARKEVKVEDGQQ

SEQ ID NO: 7 - protein, 261 aa, Streptococcus pneumoniae
  Pneumococcus Sortase C2
MSRYYYRIESNEVIKEFDETVSQMDKAELEERWRLAQAFNATLKPSEILDPFTEQEKKKGVSEYANMLKVHERIGYVEIPAIDQEIPMYVGTSEDILQKGAGLLEGASLPVGGENTHTVITAHRGLPTAELFSQLDKMKKGDIFYLHVLDQVLAYQVDQIVTVEPNDFEPVLIQHGEDYATLLTCTPYMINSHRLLVRGKRIPYTAPIAERNRAVRERGQFWLWLLLGAMAVILLLLYRVYRNRRIVKGLEKQLEGRHVKD

SEQ ID NO: 8 - protein, 283 aa, Streptococcus pneumoniae
  Pneumococcus Sortase C3
MSRTKLRALLGYLLMLVACLIPIYCFGQMVLQSLGQVKGHATFVKSMTTEMYQEQQNHSLAYNQRLASQNRIVDPFLAEGYEVNYQVSDDPDAVYGYLSIPSLEIMEPVYLGADYHHLGMGLAHVDGTPLPLDGTGIRSVIAGHRAEPSHVFFRHLDQLKVGDALYYDNGQEIVEYQMMDTEIILPSEWEKLESVSSKNIMTLITCDPIPTFNKRLLVNFERVAVYQKSDPQTAAVARVAFTKEGQSVSRVATSQWLYRGLVVLAFLGILFVLWKLARLLRGK

SEQ ID NO: 9 - protein, 251 aa, Streptococcus pyogenes
  GAS Sortase C1
MECYRDRQLLSTYHKQVTQKKPSEMEEVWQKAKAYNARLGIQPVPDAFSFRDGIHDKNYESLLQIENNDIMGYVEVPSIKVTLPIYHYTTDEVLTKGAGHLFGSALPVGGDGTHTVISAHRGLPSAEMFTNLNLVKKGDTFYFRVLNKVLAYKVDQILTVEPDQVTSLSGVMGKDYATLVTCTPYGVNTKRLLVRGHRIAYHYKKYQQAKKAMKLVDKSRMWAEVVCAAFGVVIAIILVFMYSRVSAKKSK

SEQ ID NO: 10 - protein, 256 aa, Streptococcus agalactiae
  GBS Sortase C1 of PI-1 deletion mutant
IVSQVMYFQASHANINAFKEAVTKIDRVEINRRLELAYAYNASIAGAKTNGEYEYARMLEVKEQIGHVIIPRINQDIPIYAGSAEENLQRGVGHLEGTSLPVGGESTHAVLTAHRGLPTAKLFTNLDKVTVGDRFYIEHIGGKIAYQVDQIKVIAPDQLEDLYVIQGEDHVTLLTCTPYMINSHRLLVRGKRIPYVEKTVQKDSKTFRQQQYLTYAMWVVVGLILLSLLIWFKKTKQKKRRKNEKAASQNSHNNSK

SEQ ID NO: 11 - protein, 225 aa, Streptococcus agalactiae
  GBS Sortase C2 of PI-1 deletion mutant
ASHQNINQFKREVAKIDTNTVERRIALANAYNETLSREYARMLEVHEQIGHVAIPSIGVDIPIYAGTSETVLQKGSGHLEGTSLPVGGLSTHSVLTAHRGLPTARLFTDLNKVKKGQIFYVTNIKETLAYKVVSIKVVDPTALSEVKIVNGKDYITLLTCTPYMINSHRLLVKGERIPYDSTEAEKHKEQTVQDYRLSLVLKILLVLLIGLFIVIMMRRWMQHRQ

SEQ ID NO: 12 - protein, 234 aa, Streptococcus agalactiae
  GBS Sortase C1 of PI-2a deletion mutant
ESNNQTQDFERAAKKLSQKEINRRMALAQAYNDSLNNVEYARMLEVSEKIGIISVPKIGQKLPIFAGSSQEVLSKGAGHLEGTSLPIGGNSTHTVITAHSGIPDKELFSNLKKLKKGDKFYIQNIKETIAYQVDQIKVVTPDNFSDLLVVPGHDYATLLTCTPIMVNTHRLLVRGHRIPYKGPIDEKLIKDGHLNTIYRYLFYISLVIIAWLLWLIKRQRQKNRLSSVRKGIES

SEQ ID NO: 13 - protein, 232 aa, Streptococcus agalactiae
  GBS Sortase C2 of PI-2a deletion mutant
KANNQVTNFDNQTQKLNAKEINRRFELAKAYNRTLDPEYAHMLEITEMIGYIDIPSIKQKLPIYAGTTSSVLEKGSGHLEGTSLPIGGKSSHTVITAHRGLPKAKLFTDLDKLKKGKIFYIHNIKEVLAYKVDQISVVKPDNFSKLLVVKGKDYATLLTCTPYSINSHRLLVRGHRIKYVPPVKEKNYLMKELQTHYKLYFLLSILVILILVALLLYLKRKFKERKRKGNQK

SEQ ID NO: 14 - protein, 238 aa, Streptococcus agalactiae
  GBS Sortase C1 of PI-2b deletion mutant
HQSRAIMDYQDRVTHMDENDYKKIINRAKEYNKQFNSQLAIDKTGNMGYISIPKINIKLPLYHGTSEKVLQTSIGHLEGSSLPIGGDSTHSILSGHRGLPSSRLFSDLDKLKVGDHWTVSILNETYTYQVDQIRTVKPDDLRDLQIVKGKDYQTLVTCTPYGVNTHRLLVRGHRVPNDNGNALVVAEAIQIEPIYIAPFIAIFLTLILLLISLEVTRRARQRKKILKQAMRKEENNDL

SEQ ID NO: 15 - protein, 244 aa, Streptococcus pneumoniae
  Pneumococcus sortase C2 deletion mutant
ESNQQIADFDKEKATLDEADIDERMKLAQAFNDSLEYARMLEIHERMGHVEIPVIDVDLPVYAGTAEEVLQQGAGHLEGTSLPIGGNSTHAVITAHTGLPTAKMFTDLTKLKVGDKFYVHNIKEVMAYQVDQVKVIEPTNFDDLLIVPGHDYVTLLTCTPYMINTHRLLVRGHRIPYVAEVEEEFIAANKLSHLYRYLFYVAVGLIVILLWIIRRLRKKKKQPEKALKALKAARKEVKVEDGQQ

SEQ ID NO: 16 - protein, 235 aa, Streptococcus pneumoniae
  Pneumococcus sortase C2 deletion mutant
ESNEVIKEFDETVSQMDKAELEERWRLAQAFNATLKEYANMLKVHERIGYVEIPAIDQEIPMYVGTSEDILQKGAGLLEGASLPVGGENTHTVITAHRGLPTAELFSQLDKMKKGDIFYLHVLDQVLAYQVDQIVTVEPNDFEPVLIQHGEDYATLLTCTPYMINSHRLLVRGKRIPYTAPIAERNRAVRERGQFWLWLLLGAMAVILLLLYRVYRNRRIVKGLEKQLEGRHVKD

SEQ ID NO: 17 - protein, 240 aa, Streptococcus pneumoniae
  Pneumococcus sortase C3 deletion mutant
QSLGQVKGHATFVKSMTTEMYQEQQNHSLAYNQRLASQEVNYQVSDDPDAVYGYLSIPSLEIMEPVYLGADYHHLGMGLAHVDGTPLPLDGTGIRSVIAGHRAEPSHVFFRHLDQLKVGDALYYDNGQEIVEYQMMDTEIILPSEWEKLESVSSKNIMTLITCDPIPTFNKRLLVNFERVAVYQKSDPQTAAVARVAFTKEGQSVSRVATSQWLYRGLVVLAFLGILFVLWKLARLLRGK

SEQ ID NO: 18 - protein, 229 aa, Streptococcus pyogenes
  GAS Sortase C1 deletion mutant
RDRQLLSTYHKQVTQKKPSEMEEVWQKAKAYNARLNYESLLQIENNDIMGYVEVPSIKVTLPIYHYTTDEVLTKGAGHLFGSALPVGGDGTHTVISAHRGLPSAEMFTNLNLVKKGDTFYFRVLNKVLAYKVDQILTVEPDQVTSLSGVMGKDYATLVTCTPYGVNTKRLLVRGHRIAYHYKKYQQAKKAMKLVDKSRMWAEVVCAAFGVVIAIILVFMYSRVSAKKSK

SEQ ID NO: 19 - protein, 203 aa, Streptococcus agalactiae
  GBS Sortase C1 of PI-1 deletion mutant
EYARMLEVKEQIGHVIIPRINQDIPIYAGSAEENLQRGVGHLEGTSLPVGGESTHAVLTAHRGLPTAKLFTNLDKVTVGDRFYIEHIGGKIAYQVDQIKVIAPDQLEDLYVIQGEDHVTLLTCTPYMINSHRLLVRGKRIPYVEKTVQKDSKTFRQQQYLTYAMWVVVGLILLSLLIWFKKTKQKKRRKNEKAASQNSHNNSK

SEQ ID NO: 20 - protein, 188 aa, Streptococcus agalactiae
  GBS sortase C2 of PI-1 deletion mutant
EYARMLEVHEQIGHVAIPSIGVDIPIYAGTSETVLQKGSGHLEGTSLPVGGLSTHSVLTAHRGLPTARLFTDLNKVKKGQIFYVTNIKETLAYKVVSIKVVDPTALSEVKIVNGKDYITLLTCTPYMINSHRLLVKGERIPYDSTEAEKHKEQTVQDYRLSLVLKILLVLLIGLFIVIMMRRWMQHRQ

SEQ ID NO: 21 - protein, 196 aa, Streptococcus agalactiae
  GBS sortase C1 of PI-2a deletion mutant
EYARMLEVSEKIGIISVPKIGQKLPIFAGSSQEVLSKGAGHLEGTSLPIGGNSTHTVITAHSGIPDKELFSNLKKLKKGDKFYIQNIKETIAYQVDQIKVVTPDNFSDLLVVPGHDYATLLTCTPIMVNTHRLLVRGHRIPYKGPIDEKLIKDGHLNTIYRYLFYISLVIIAWLLWLIKRQRQKNRLSSVRKGIES

SEQ ID NO: 22 - protein, 195 aa, Streptococcus agalactiae
  GBS sortase C2 of PI-2a deletion mutant
EYAHMLEITEMIGYIDIPSIKQKLPIYAGTTSSVLEKGSGHLEGTSLPIGGKSSHTVITAHRGLPKAKLFTDLDKLKKGKIFYIHNIKEVLAYKVDQISVVKPDNFSKLLVVKGKDYATLLTCTPYSINSHRLLVRGHRIKYVPPVKEKNYLMKELQTHYKLYFLLSILVILILVALLLYLKRKFKERKRKGNQK

SEQ ID NO: 23 - protein, 203 aa, Streptococcus agalactiae
  GBS sortase C1 of PI-2b deletion mutant
NSQLAIDKTGNMGYISIPKINIKLPLYHGTSEKVLQTSIGHLEGSSLPIGGDSTHSILSGHRGLPSSRLFSDLDKLKVGDHWTVSILNETYTYQVDQIRTVKPDDLRDLQIVKGKDYQTLVTCTPYGVNTHRLLVRGHRVPNDNGNALVVAEAIQIEPIYIAPFIAIFLTLILLLISLEVTRRARQRKKILKQAMRKEENNDL

SEQ ID NO: 24 - protein, 209 aa, Streptococcus pneumoniae
  Pneumococcus sortase C1 deletion mutant
EYARMLEIHERMGHVEIPVIDVDLPVYAGTAEEVLQQGAGHLEGTSLPIGGNSTHAVITAHTGLPTAKMFTDLTKLKVGDKFYVHNIKEVMAYQVDQVKVIEPTNFDDLLIVPGHDYVTLLTCTPYMINTHRLLVRGHRIPYVAEVEEEFIAANKLSHLYRYLFYVAVGLIVILLWIIRRLRKKKKQPEKALKALKAARKEVKVEDGQQ

SEQ ID NO: 25 - protein, 199 aa, Streptococcus pneumoniae
  Pneumococcus sortase C2 deletion mutant
EYANMLKVHERIGYVEIPAIDQEIPMYVGTSEDILQKGAGLLEGASLPVGGENTHTVITAHRGLPTAELFSQLDKMKKGDIFYLHVLDQVLAYQVDQIVTVEPNDFEPVLIQHGEDYATLLTCTPYMINSHRLLVRGKRIPYTAPIAERNRAVRERGQFWLWLLLGAMAVILLLLYRVYRNRRIVKGLEKQLEGRHVKD

SEQ ID NO: 26 - protein, 202 aa, Streptococcus pneumoniae
  Pneumococcus sortase C3 deletion mutant
EVNYQVSDDPDAVYGYLSIPSLEIMEPVYLGADYHHLGMGLAHVDGTPLPLDGTGIRSVIAGHRAEPSHVFFRHLDQLKVGDALYYDNGQEIVEYQMMDTEIILPSEWEKLESVSSKNIMTLITCDPIPTFNKRLLVNFERVAVYQKSDPQTAAVARVAFTKEGQSVSRVATSQWLYRGLVVLAFLGILFVLWKLARLLRGK

SEQ ID NO: 27 - protein, 197 aa, Streptococcus agalactiae
  GAS sortase C1 deletion mutant
HDKNYESLLQIENNDIMGYVEVPSIKVTLPIYHYTTDEVLTKGAGHLFGSALPVGGDGTHTVISAHRGLPSAEMFTNLNLVKKGDTFYFRVLNKVLAYKVDQILTVEPDQVTSLSGVMGKDYATLVTCTPYGVNTKRLLVRGHRIAYHYKKYQQAKKAMKLVDKSRMWAEVVCAAFGVVIAIILVFMYSRVSAKKSK

SEQ ID NO: 28 - protein, 270 aa, Streptococcus pyogenes
  GBS sortase C1 of PI-1 deletion mutant
IVSQVMYFQASHANINAFKEAVTKIDRVEINRRLELAYAYNASIAGAKTNGEYPALKSAEQKQAGVVEYARMLEVKEQIGHVIIPRINQDIPIYAGSAEENLQRGVGHLEGTSLPVGGESTHAVLTAHRGLPTAKLFTNLDKVTVGDRFYIEHIGGKIAYQVDQIKVIAPDQLEDLYVIQGEDHVTLLTCTPYMINSHRLLVRGKRIPYVEKTVQKDSKTFRQQQYLTYAMWVVVGLILLSLLIWFKKTKQKKRRKNEKAASQNSHNNSK

SEQ ID NO: 29 - protein, 239 aa, Streptococcus pyogenes
  GBS sortase C2 of PI-1
ASHQNINQFKREVAKIDTNTVERRIALANAYNETLSRNPLLITSKQKEGLREYARMLEVHEQIGHVAIPSIGVDIPIYAGTSETVLQKGSGHLEGTSLPVGGLSTHSVLTAHRGLPTARLFTDLNKVKKGQIFYVTNIKETLAYKVVSIKVVDPTALSEVKIVNGKDYITLLTCTPYMINSHRLLVKGERIPYDSTEAEKHKEQTVQDYRLSLVLKILLVLLIGLFIVIMMRRWMQHRQ

SEQ ID NO: 30 - protein, 247 aa, Streptococcus pyogenes
  GBS sortase C1 of PI-2a deletion mutant
ESNNQTQDFERAAKKLSQKEINRRMALAQAYNDSLNNVHLEEKKRIQKGIAEYARMLEVSEKIGIISVPKIGQKLPIFAGSSQEVLSKGAGHLEGTSLPIGGNSTHTVITAHSGIPDKELFSNLKKLKKGDKFYIQNIKETIAYQVDQIKVVTPDNFSDLLVVPGHDYATLLTCTPIMVNTHRLLVRGHRIPYKGPIDEKLIKDGHLNTIYRYLFYISLVIIAWLLWLIKRQRQKNRLSSVRKGIES

SEQ ID NO: 31 - protein, 245 aa, Streptococcus pyogenes
  GBS sortase C2 of PI-2a deletion mutant
KANNQVTNFDNQTQKLNAKEINRRFELAKAYNRTLDPSRLSTEKEKKGIAEYAHMLEITEMIGYIDIPSIKQKLPIYAGTTSSVLEKGSGHLEGTSLPIGGKSSHTVITAHRGLPKAKLFTDLDKLKKGKIFYIHNIKEVLAYKVDQISVVKPDNFSKLLVVKGKDYATLLTCTPYSINSHRLLVRGHRIKYVPPVKEKNYLMKELQTHYKLYFLLSILVILILVALLLYLKRKFKERKRKGNQK

SEQ ID NO: 32 - protein, 252 aa, Streptococcus pyogenes
  GBS sortase C1 of PI-2b deletion mutant
HQSRAIMDYQDRVTHMDENDYKKIINRAKEYNKQFKTSGHMTSQERLDYNSQLAIDKTGNMGYISIPKINIKLPLYHGTSEKVLQTSIGHLEGSSLPIGGDSTHSILSGHRGLPSSRLFSDLDKLKVGDHWTVSILNETYTYQVDQIRTVKPDDLRDLQIVKGKDYQTLVTCTPYGVNTHRLLVRGHRVPNDNGNALVVAEAIQIEPIYIAPFIAIFLTLILLLISLEVTRRARQRKKILKQAMRKEENNDL

SEQ ID NO: 33 - protein, 260 aa, Streptococcus pneumoniae
  Pneumococcus sortase C1 deletion mutant
ESNQQIADFDKEKATLDEADIDERMKLAQAFNDSLNNVVSGSEEMKKKGRAEYARMLEIHERMGHVEIPVIDVDLPVYAGTAEEVLQQGAGHLEGTSLPIGGNSTHAVITAHTGLPTAKMFTDLTKLKVGDKFYVHNIKEVMAYQVDQVKVIEPTNFDDLLIVPGHDYVTLLTCTPYMINTHRLLVRGHRIPYVAEVEEEFIAANKLSHLYRYLFYVAVGLIVILLWIIRRLRKKKKQPEKALKALKAARKEVKVEDGQQ

SEQ ID NO: 34 - protein, 250 aa, Streptococcus pneumoniae
  Pneumococcus sortase C2 deletion mutant
ESNEVIKEFDETVSQMDKAELEERWRLAQAFNATLKPSEILTEQEKKKGVSEYANMLKVHERIGYVEIPAIDQEIPMYVGTSEDILQKGAGLLEGASLPVGGENTHTVITAHRGLPTAELFSQLDKMKKGDIFYLHVLDQVLAYQVDQIVTVEPNDFEPVLIQHGEDYATLLTCTPYMINSHRLLVRGKRIPYTAPIAERNRAVRERGQFWLWLLLGAMAVILLLLYRVYRNRRIVKGLEKQLEGRHVKD

SEQ ID NO: 35 - protein, 249 aa, Streptococcus pneumoniae
  Pneumococcus sortase C3
QSLGQVKGHATFVKSMTTEMYQEQQNHSLAYNQRLASQNRIVLAEGYEVNYQVSDDPDAVYGYLSIPSLEIMEPVYLGADYHHLGMGLAHVDGTPLPLDGTGIRSVIAGHRAEPSHVFFRHLDQLKVGDALYYDNGQEIVEYQMMDTEIILPSEWEKLESVSSKNIMTLITCDPIPTFNKRLLVNFERVAVYQKSDPQTAAVARVAFTKEGQSVSRVATSQWLYRGLVVLAFLGILFVLWKLARLLRGK

SEQ ID NO: 36 - protein, 244 aa, Streptococcus agalactiae
  GAS sortase C1 deletion mutant
RDRQLLSTYHKQVTQKKPSEMEEVWQKAKAYNARLGIQPVPSFRDGIHDKNYESLLQIENNDIMGYVEVPSIKVTLPIYHYTTDEVLTKGAGHLFGSALPVGGDGTHTVISAHRGLPSAEMFTNLNLVKKGDTFYFRVLNKVLAYKVDQILTVEPDQVTSLSGVMGKDYATLVTCTPYGVNTKRLLVRGHRIAYHYKKYQQAKKAMKLVDKSRMWAEVVCAAFGVVIAIILVFMYSRVSAKKSK

SEQ ID NO: 37 - protein, 273 aa, Streptococcus pyogenes
  GBS sortase C1 of PI-1 substitution mutant
IVSQVMYFQASHANINAFKEAVTKIDRVEINRRLELAYAYNASIAGAKTNGEYPALKAPYSAEQKQAGVVEYARMLEVKEQIGHVIIPRINQDIPIYAGSAEENLQRGVGHLEGTSLPVGGESTHAVLTAHRGLPTAKLFTNLDKVTVGDRFYIEHIGGKIAYQVDQIKVIAPDQLEDLYVIQGEDHVTLLTCTPYMINSHRLLVRGKRIPYVEKTVQKDSKTFRQQQYLTYAMWVVVGLILLSLLIWFKKTKQKKRRKNEKAASQNSHNNSK

SEQ ID NO: 38 - protein, 242 aa, Streptococcus pyogenes
  GBS sortase C2 of PI-1 substitution mutant
ASHQNINQFKREVAKIDTNTVERRIALANAYNETLSRNPLLIAPFTSKQKEGLREYARMLEVHEQIGHVAIPSIGVDIPIYAGTSETVLQKGSGHLEGTSLPVGGLSTHSVLTAHRGLPTARLFTDLNKVKKGQIFYVTNIKETLAYKVVSIKVVDPTALSEVKIVNGKDYITLLTCTPYMINSHRLLVKGERIPYDSTEAEKHKEQTVQDYRLSLVLKILLVLLIGLFIVIMMRRWMQHRQ

SEQ ID NO: 39 - protein, 250 aa, Streptococcus pyogenes
  GBS sortase C1 of PI-2a substitution mutant
ESNNQTQDFERAAKKLSQKEINRRMALAQAYNDSLNNVHLEAPYEKKRIQKGIAEYARMLEVSEKIGIISVPKIGQKLPIFAGSSQEVLSKGAGHLEGTSLPIGGNSTHTVITAHSGIPDKELFSNLKKLKKGDKFYIQNIKETIAYQVDQIKVVTPDNFSDLLVVPGHDYATLLTCTPIMVNTHRLLVRGHRIPYKGPIDEKLIKDGHLNTIYRYLFYISLVIIAWLLWLIKRQRQKNRLSSVRKGIES

SEQ ID NO: 40 - protein, 248 aa, Streptococcus pyogenes
  GBS sortase C2 of PI-2a substitution mutant
KANNQVTNFDNQTQKLNAKEINRRFELAKAYNRTLDPSRLSAPYTEKEKKGIAEYAHMLEITEMIGYIDIPSIKQKLPIYAGTTSSVLEKGSGHLEGTSLPIGGKSSHTVITAHRGLPKAKLFTDLDKLKKGKIFYIHNIKEVLAYKVDQISVVKPDNFSKLLVVKGKDYATLLTCTPYSINSHRLLVRGHRIKYVPPVKEKNYLMKELQTHYKLYFLLSILVILILVALLLYLKRKFKERKRKGNQK

SEQ ID NO: 41 - protein, 255 aa, Streptococcus pyogenes
  GBS sortase C1 of PI-2b substitution mutant
HQSRAIMDYQDRVTHMDENDYKKIINRAKEYNKQFKTSGAKWHMTSQERLDYNSQLAIDKTGNMGYISIPKINIKLPLYHGTSEKVLQTSIGHLEGSSLPIGGDSTHSILSGHRGLPSSRLFSDLDKLKVGDHWTVSILNETYTYQVDQIRTVKPDDLRDLQIVKGKDYQTLVTCTPYGVNTHRLLVRGHRVPNDNGNALVVAEAIQIEPIYIAPFIAIFLTLILLLISLEVTRRARQRKKILKQAMRKEENNDL

SEQ ID NO: 42 - protein, 263 aa, Streptococcus pneumoniae
  Pneumococcus sortase C1 substitution mutant
ESNQQIADFDKEKATLDEADIDERMKLAQAFNDSLNNVVSGAPWSEEMKKKGRAEYARMLEIHERMGHVEIPVIDVDLPVYAGTAEEVLQQGAGHLEGTSLPIGGNSTHAVITAHTGLPTAKMFTDLTKLKVGDKFYVHNIKEVMAYQVDQVKVIEPTNFDDLLIVPGHDYVTLLTCTPYMINTHRLLVRGHRIPYVAEVEEEFIAANKLSHLYRYLFYVAVGLIVILLWIIRRLRKKKKQPEKALKALKAARKEVKVEDGQQ

SEQ ID NO: 43 - protein, 253 aa, Streptococcus pneumoniae
  Pneumococcus sortase C2 substitution mutant
ESNEVIKEFDETVSQMDKAELEERWRLAQAFNATLKPSEILAPFTEQEKKKGVSEYANMLKVHERIGYVEIPAIDQEIPMYVGTSEDILQKGAGLLEGASLPVGGENTHTVITAHRGLPTAELFSQLDKMKKGDIFYLHVLDQVLAYQVDQIVTVEPNDFEPVLIQHGEDYATLLTCTPYMINSHRLLVRGKRIPYTAPIAERNRAVRERGQFWLWLLLGAMAVILLLLYRVYRNRRIVKGLEKQLEGRHVKD

SEQ ID NO: 44 - protein, 252 aa, Streptococcus pneumoniae
  Pneumococcus sortase C3 substitution mutant
QSLGQVKGHATFVKSMTTEMYQEQQNHSLAYNQRLASQNRIVAPFLAEGYEVNYQVSDDPDAVYGYLSIPSLEIMEPVYLGADYHHLGMGLAHVDGTPLPLDGTGIRSVIAGHRAEPSHVFFRHLDQLKVGDALYYDNGQEIVEYQMMDTEIILPSEWEKLESVSSKNIMTLITCDPIPTFNKRLLVNFERVAVYQKSDPQTAAVARVAFTKEGQSVSRVATSQWLYRGLVVLAFLGILFVLWKLARLLRGK

SEQ ID NO: 45 - protein, 247 aa, Streptococcus agalactiae
  GAS sortase C1 substitution mutant
RDRQLLSTYHKQVTQKKPSEMEEVWQKAKAYNARLGIQPVPAAFSFRDGIHDKNYESLLQIENNDIMGYVEVPSIKVTLPIYHYTTDEVLTKGAGHLFGSALPVGGDGTHTVISAHRGLPSAEMFTNLNLVKKGDTFYFRVLNKVLAYKVDQILTVEPDQVTSLSGVMGKDYATLVTCTPYGVNTKRLLVRGHRIAYHYKKYQQAKKAMKLVDKSRMWAEVVCAAFGVVIAIILVFMYSRVSAKKSK

SEQ ID NO: 46 - protein, 273 aa, Streptococcus agalactiae
  GBS sortase C1 of PI-1 substitution mutant
IVSQVMYFQASHANINAFKEAVTKIDRVEINRRLELAYAYNASIAGAKTNGEYPALKDPASAEQKQAGVVEYARMLEVKEQIGHVIIPRINQDIPIYAGSAEENLQRGVGHLEGTSLPVGGESTHAVLTAHRGLPTAKLFTNLDKVTVGDRFYIEHIGGKIAYQVDQIKVIAPDQLEDLYVIQGEDHVTLLTCTPYMINSHRLLVRGKRIPYVEKTVQKDSKTFRQQQYLTYAMWVVVGLILLSLLIWFKKTKQKKRRKNEKAASQNSHNNSK

SEQ ID NO: 47 - protein, 242 aa, Streptococcus agalactiae
  GBS sortase C2 of PI-1 substitution mutant
ASHQNINQFKREVAKIDTNTVERRIALANAYNETLSRNPLLIDPATSKQKEGLREYARMLEVHEQIGHVAIPSIGVDIPIYAGTSETVLQKGSGHLEGTSLPVGGLSTHSVLTAHRGLPTARLFTDLNKVKKGQIFYVTNIKETLAYKVVSIKVVDPTALSEVKIVNGKDYITLLTCTPYMINSHRLLVKGERIPYDSTEAEKHKEQTVQDYRLSLVLKILLVLLIGLFIVIMMRRWMQHRQ

SEQ ID NO: 48 - protein, 250 aa, Streptococcus agalactiae
  GBS sortase C1 of PI-2a substitution mutant
ESNNQTQDFERAAKKLSQKEINRRMALAQAYNDSLNNVHLEDPAEKKRIQKGIAEYARMLEVSEKIGIISVPKIGQKLPIFAGSSQEVLSKGAGHLEGTSLPIGGNSTHTVITAHSGIPDKELFSNLKKLKKGDKFYIQNIKETIAYQVDQIKVVTPDNFSDLLVVPGHDYATLLTCTPIMVNTHRLLVRGHRIPYKGPIDEKLIKDGHLNTIYRYLFYISLVIIAWLLWLIKRQRQKNRLSSVRKGIES

SEQ ID NO: 49 - protein, 248 aa, Streptococcus agalactiae
  GBS sortase C2 of PI-2a substitution mutant
KANNQVTNFDNQTQKLNAKEINRRFELAKAYNRTLDPSRLSDPATEKEKKGIAEYAHMLEITEMIGYIDIPSIKQKLPIYAGTTSSVLEKGSGHLEGTSLPIGGKSSHTVITAHRGLPKAKLFTDLDKLKKGKIFYIHNIKEVLAYKVDQISVVKPDNFSKLLVVKGKDYATLLTCTPYSINSHRLLVRGHRIKYVPPVKEKNYLMKELQTHYKLYFLLSILVILILVALLLYLKRKFKERKRKGNQK

SEQ ID NO: 50 - protein, 255 aa, Streptococcus agalactiae
  GBS sortase C1 of PI-2b substitution mutant
HQSRAIMDYQDRVTHMDENDYKKIINRAKEYNKQFKTSGMKAHMTSQERLDYNSQLAIDKTGNMGYISIPKINIKLPLYHGTSEKVLQTSIGHLEGSSLPIGGDSTHSILSGHRGLPSSRLFSDLDKLKVGDHWTVSILNETYTYQVDQIRTVKPDDLRDLQIVKGKDYQTLVTCTPYGVNTHRLLVRGHRVPNDNGNALVVAEAIQIEPIYIAPFIAIFLTLILLLISLEVTRRARQRKKILKQAMRKEENNDL

SEQ ID NO: 51 - protein, 263 aa, Streptococcus pneumoniae
  Pneumococcus sortase C1 substitution mutant
ESNQQIADFDKEKATLDEADIDERMKLAQAFNDSLNNVVSGDPASEEMKKKGRAEYARMLEIHERMGHVEIPVIDVDLPVYAGTAEEVLQQGAGHLEGTSLPIGGNSTHAVITAHTGLPTAKMFTDLTKLKVGDKFYVHNIKEVMAYQVDQVKVIEPTNFDDLLIVPGHDYVTLLTCTPYMINTHRLLVRGHRIPYVAEVEEEFIAANKLSHLYRYLFYVAVGLIVILLWIIRRLRKKKKQPEKALKALKAARKEVKVEDGQQ

SEQ ID NO: 52 - protein, 253 aa, Streptococcus pneumoniae
  Pneumococcus sortase C2 substitution mutant
ESNEVIKEFDETVSQMDKAELEERWRLAQAFNATLKPSEILDPATEQEKKKGVSEYANMLKVHERIGYVEIPAIDQEIPMYVGTSEDILQKGAGLLEGASLPVGGENTHTVITAHRGLPTAELFSQLDKMKKGDIFYLHVLDQVLAYQVDQIVTVEPNDFEPVLIQHGEDYATLLTCTPYMINSHRLLVRGKRIPYTAPIAERNRAVRERGQFWLWLLLGAMAVILLLLYRVYRNRRIVKGLEKQLEGRHVKD

SEQ ID NO: 53 - protein, 252 aa, Streptococcus pneumoniae
  Pneumococcus sortase C3 substitution mutant
QSLGQVKGHATFVKSMTTEMYQEQQNHSLAYNQRLASQNRIVDPALAEGYEVNYQVSDDPDAVYGYLSIPSLEIMEPVYLGADYHHLGMGLAHVDGTPLPLDGTGIRSVIAGHRAEPSHVFFRHLDQLKVGDALYYDNGQEIVEYQMMDTEIILPSEWEKLESVSSKNIMTLITCDPIPTFNKRLLVNFERVAVYQKSDPQTAAVARVAFTKEGQSVSRVATSQWLYRGLVVLAFLGILFVLWKLARLLRGK

SEQ ID NO: 54 - protein, 247 aa, Streptococcus pyogenes
  GAS sortase C1 substitution mutant
RDRQLLSTYHKQVTQKKPSEMEEVWQKAKAYNARLGIQPVPDAASFRDGIHDKNYESLLQIENNDIMGYVEVPSIKVTLPIYHYTTDEVLTKGAGHLFGSALPVGGDGTHTVISAHRGLPSAEMFTNLNLVKKGDTFYFRVLNKVLAYKVDQILTVEPDQVTSLSGVMGKDYATLVTCTPYGVNTKRLLVRGHRIAYHYKKYQQAKKAMKLVDKSRMWAEVVCAAFGVVIAIILVFMYSRVSAKKSK

SEQ ID NO: 55 - protein, 273 aa, Streptococcus agalactiae
  GBS sortase C1 of PI-1 substitution mutant
IVSQVMYFQASHANINAFKEAVTKIDRVEINRRLELAYAYNASIAGAKTNGEYPALKAPASAEQKQAGVVEYARMLEVKEQIGHVIIPRINQDIPIYAGSAEENLQRGVGHLEGTSLPVGGESTHAVLTAHRGLPTAKLFTNLDKVTVGDRFYIEHIGGKIAYQVDQIKVIAPDQLEDLYVIQGEDHVTLLTCTPYMINSHRLLVRGKRIPYVEKTVQKDSKTFRQQQYLTYAMWVVVGLILLSLLIWFKKTKQKKRRKNEKAASQNSHNNSK

SEQ ID NO: 56 - protein, 242 aa, Streptococcus agalactiae
  GBS sortase C2 of PI-1 substitution mutant
ASHQNINQFKREVAKIDTNTVERRIALANAYNETLSRNPLLIAPATSKQKEGLREYARMLEVHEQIGHVAIPSIGVDIPIYAGTSETVLQKGSGHLEGTSLPVGGLSTHSVLTAHRGLPTARLFTDLNKVKKGQIFYVTNIKETLAYKVVSIKVVDPTALSEVKIVNGKDYITLLTCTPYMINSHRLLVKGERIPYDSTEAEKHKEQTVQDYRLSLVLKILLVLLIGLFIVIMMRRWMQHRQ

SEQ ID NO: 57 - protein, 250 aa, Streptococcus agalactiae
  GBS sortase C1 of PI-2a substitution mutant
ESNNQTQDFERAAKKLSQKEINRRMALAQAYNDSLNNVHLEAPAEKKRIQKGIAEYARMLEVSEKIGIISVPKIGQKLPIFAGSSQEVLSKGAGHLEGTSLPIGGNSTHTVITAHSGIPDKELFSNLKKLKKGDKFYIQNIKETIAYQVDQIKVVTPDNFSDLLVVPGHDYATLLTCTPIMVNTHRLLVRGHRIPYKGPIDEKLIKDGHLNTIYRYLFYISLVIIAWLLWLIKRQRQKNRLSSVRKGIES

SEQ ID NO: 58 - protein, 248 aa, Streptococcus agalactiae
  GBS sortase C2 of PI-2a substitution mutant
KANNQVTNFDNQTQKLNAKEINRRFELAKAYNRTLDPSRLSAPATEKEKKGIAEYAHMLEITEMIGYIDIPSIKQKLPIYAGTTSSVLEKGSGHLEGTSLPIGGKSSHTVITAHRGLPKAKLFTDLDKLKKGKIFYIHNIKEVLAYKVDQISVVKPDNFSKLLVVKGKDYATLLTCTPYSINSHRLLVRGHRIKYVPPVKEKNYLMKELQTHYKLYFLLSILVILILVALLLYLKRKFKERKRKGNQK

SEQ ID NO: 59 - protein, 255 aa, Streptococcus agalactiae
  GBS sortase C1 of PI-2b substitution mutant
HQSRAIMDYQDRVTHMDENDYKKIINRAKEYNKQFKTSGAKAHMTSQERLDYNSQLAIDKTGNMGYISIPKINIKLPLYHGTSEKVLQTSIGHLEGSSLPIGGDSTHSILSGHRGLPSSRLFSDLDKLKVGDHWTVSILNETYTYQVDQIRTVKPDDLRDLQIVKGKDYQTLVTCTPYGVNTHRLLVRGHRVPNDNGNALVVAEAIQIEPIYIAPFIAIFLTLILLLISLEVTRRARQRKKILKQAMRKEENNDL

SEQ ID NO: 60 - protein, 263 aa, Streptococcus pneumoniae
  Pneumococcus sortase C1 substitution mutant
ESNQQIADFDKEKATLDEADIDERMKLAQAFNDSLNNVVSGAPASEEMKKKGRAEYARMLEIHERMGHVEIPVIDVDLPVYAGTAEEVLQQGAGHLEGTSLPIGGNSTHAVITAHTGLPTAKMFTDLTKLKVGDKFYVHNIKEVMAYQVDQVKVIEPTNFDDLLIVPGHDYVTLLTCTPYMINTHRLLVRGHRIPYVAEVEEEFIAANKLSHLYRYLFYVAVGLIVILLWIIRRLRKKKKQPEKALKALKAARKEVKVEDGQQ

SEQ ID NO: 61 - protein, 253 aa, Streptococcus pneumoniae
  Pneumococcus sortase C2 substitution mutant
ESNEVIKEFDETVSQMDKAELEERWRLAQAFNATLKPSEILAPATEQEKKKGVSEYANMLKVHERIGYVEIPAIDQEIPMYVGTSEDILQKGAGLLEGASLPVGGENTHTVITAHRGLPTAELFSQLDKMKKGDIFYLHVLDQVLAYQVDQIVTVEPNDFEPVLIQHGEDYATLLTCTPYMINSHRLLVRGKRIPYTAPIAERNRAVRERGQFWLWLLLGAMAVILLLLYRVYRNRRIVKGLEKQLEGRHVKD

SEQ ID NO: 62 - protein, 252 aa, Streptococcus pneumoniae
  Pneumococcus sortase C3 substitution mutant
QSLGQVKGHATFVKSMTTEMYQEQQNHSLAYNQRLASQNRIVAPALAEGYEVNYQVSDDPDAVYGYLSIPSLEIMEPVYLGADYHHLGMGLAHVDGTPLPLDGTGIRSVIAGHRAEPSHVFFRHLDQLKVGDALYYDNGQEIVEYQMMDTEIILPSEWEKLESVSSKNIMTLITCDPIPTFNKRLLVNFERVAVYQKSDPQTAAVARVAFTKEGQSVSRVATSQWLYRGLVVLAFLGILFVLWKLARLLRGK

SEQ ID NO: 63 - protein, 247 aa, Streptococcus pyogenes
  GAS sortase C1 substitution mutant
RDRQLLSTYHKQVTQKKPSEMEEVWQKAKAYNARLGIQPVPAAASFRDGIHDKNYESLLQIENNDIMGYVEVPSIKVTLPIYHYTTDEVLTKGAGHLFGSALPVGGDGTHTVISAHRGLPSAEMFTNLNLVKKGDTFYFRVLNKVLAYKVDQILTVEPDQVTSLSGVMGKDYATLVTCTPYGVNTKRLLVRGHRIAYHYKKYQQAKKAMKLVDKSRMWAEVVCAAFGVVIAIILVFMYSRVSAKKSK

SEQ ID NO: 64 - protein, 273 aa, Streptococcus agalactiae
  GBS sortase C1 of PI-1 substitution mutant
IVSQVMYFQASHANINAFKEAVTKIDRVEINRRLELAYAYNASIAGAKTNGEYPALKAAASAEQKQAGVVEYARMLEVKEQIGHVIIPRINQDIPIYAGSAEENLQRGVGHLEGTSLPVGGESTHAVLTAHRGLPTAKLFTNLDKVTVGDRFYIEHIGGKIAYQVDQIKVIAPDQLEDLYVIQGEDHVTLLTCTPYMINSHRLLVRGKRIPYVEKTVQKDSKTFRQQQYLTYAMWVVVGLILLSLLIWFKKTKQKKRRKNEKAASQNSHNNSK

SEQ ID NO: 65 - protein, 242 aa, Streptococcus agalactiae
  GBS sortase C2 of PI-1 substitution mutant
ASHQNINQFKREVAKIDTNTVERRIALANAYNETLSRNPLLIAAATSKQKEGLREYARMLEVHEQIGHVAIPSIGVDIPIYAGTSETVLQKGSGHLEGTSLPVGGLSTHSVLTAHRGLPTARLFTDLNKVKKGQIFYVTNIKETLAYKVVSIKVVDPTALSEVKIVNGKDYITLLTCTPYMINSHRLLVKGERIPYDSTEAEKHKEQTVQDYRLSLVLKILLVLLIGLFIVIMMRRWMQHRQ

SEQ ID NO: 66 - protein, 250 aa, Streptococcus agalactiae
  GBS sortase C1 of PI-2a substitution mutant
ESNNQTQDFERAAKKLSQKEINRRMALAQAYNDSLNNVHLEAAAEKKRIQKGIAEYARMLEVSEKIGIISVPKIGQKLPIFAGSSQEVLSKGAGHLEGTSLPIGGNSTHTVITAHSGIPDKELFSNLKKLKKGDKFYIQNIKETIAYQVDQIKVVTPDNFSDLLVVPGHDYATLLTCTPIMVNTHRLLVRGHRIPYKGPIDEKLIKDGHLNTIYRYLFYISLVIIAWLLWLIKRQRQKNRLSSVRKGIES

SEQ ID NO: 67 - protein, 248 aa, Streptococcus agalactiae
  GBS sortase C2 of PI-2a substitution mutant
KANNQVTNFDNQTQKLNAKEINRRFELAKAYNRTLDPSRLSAAATEKEKKGIAEYAHMLEITEMIGYIDIPSIKQKLPIYAGTTSSVLEKGSGHLEGTSLPIGGKSSHTVITAHRGLPKAKLFTDLDKLKKGKIFYIHNIKEVLAYKVDQISVVKPDNFSKLLVVKGKDYATLLTCTPYSINSHRLLVRGHRIKYVPPVKEKNYLMKELQTHYKLYFLLSILVILILVALLLYLKRKFKERKRKGNQK

SEQ ID NO: 68 - protein, 255 aa, Streptococcus agalactiae
  GBS sortase C1 of PI-2b substitution mutant
HQSRAIMDYQDRVTHMDENDYKKIINRAKEYNKQFKTSGAAAHMTSQERLDYNSQLAIDKTGNMGYISIPKINIKLPLYHGTSEKVLQTSIGHLEGSSLPIGGDSTHSILSGHRGLPSSRLFSDLDKLKVGDHWTVSILNETYTYQVDQIRTVKPDDLRDLQIVKGKDYQTLVTCTPYGVNTHRLLVRGHRVPNDNGNALVVAEAIQIEPIYIAPFIAIFLTLILLLISLEVTRRARQRKKILKQAMRKEENNDL

SEQ ID NO: 69 - protein, 263 aa, Streptococcus pneumoniae
  Pneumococcus sortase C1 substitution mutant
ESNQQIADFDKEKATLDEADIDERMKLAQAFNDSLNNVVSGAAASEEMKKKGRAEYARMLEIHERMGHVEIPVIDVDLPVYAGTAEEVLQQGAGHLEGTSLPIGGNSTHAVITAHTGLPTAKMFTDLTKLKVGDKFYVHNIKEVMAYQVDQVKVIEPTNFDDLLIVPGHDYVTLLTCTPYMINTHRLLVRGHRIPYVAEVEEEFIAANKLSHLYRYLFYVAVGLIVILLWIIRRLRKKKKQPEKALKALKAARKEVKVEDGQQ

SEQ ID NO: 70 - protein, 253 aa, Streptococcus pneumoniae
  Pneumococcus sortase C2 substitution mutant
ESNEVIKEFDETVSQMDKAELEERWRLAQAFNATLKPSEILAAATEQEKKKGVSEYANMLKVHERIGYVEIPAIDQEIPMYVGTSEDILQKGAGLLEGASLPVGGENTHTVITAHRGLPTAELFSQLDKMKKGDIFYLHVLDQVLAYQVDQIVTVEPNDFEPVLIQHGEDYATLLTCTPYMINSHRLLVRGKRIPYTAPIAERNRAVRERGQFWLWLLLGAMAVILLLLYRVYRNRRIVKGLEKQLEGRHVKD

SEQ ID NO: 71 - protein, 252 aa, Streptococcus pneumoniae
  Pneumococcus sortase C3 substitution mutant
QSLGQVKGHATFVKSMTTEMYQEQQNHSLAYNQRLASQNRIVAAALAEGYEVNYQVSDDPDAVYGYLSIPSLEIMEPVYLGADYHHLGMGLAHVDGTPLPLDGTGIRSVIAGHRAEPSHVFFRHLDQLKVGDALYYDNGQEIVEYQMMDTEIILPSEWEKLESVSSKNIMTLITCDPIPTFNKRLLVNFERVAVYQKSDPQTAAVARVAFTKEGQSVSRVATSQWLYRGLVVLAFLGILFVLWKLARLLRGK

SEQ ID NO: 72 - protein, 554 aa, Streptococcus agalactiae 2603V/R
  GBS80 full length
MKLSKKLLFSAAVLTMVAGSTVEPVAQFATGMSIVRAAEVSQERPAKTTVNIYKLQADSYKSEITSNGGIENKDGEVISNYAKLGDNVKGLQGVQFKRYKVKTDISVDELKKLTTVEAADAKVGTILEEGVSLPQKTNAQGLVVDALDSKSNVRYLYVEDLKNSPSNITKAYAVPFVLELPVANSTGTGFLSEINIYPKNVVTDEPKTDKDVKKLGQDDAGYTIGEEFKWFLKSTIPANLGDYEKFEITDKFADGLTYKSVGKIKIGSKTLNRDEHYTIDEPTVDNQNTLKITFKPEKFKEIAELLKGMTLVKNQDALDKATANTDDAAFLEIPVASTINEKAVLGKAIENTFELQYDHTPDKADNPKPSNPPRKPEVHTGGKRFVKKDSTETQTLGGAEFDLLASDGTAVKWTDALIKANTNKNYIAGEAVTGQPIKLKSHTDGTFEIKGLAYAVDANAEGTAVTYKLKETKAPEGYVIPDKEIEFTVSQTSYNTKPTDITVDSADATPDTIKNNKRPSIPNTGGIGTAIFVAIGAAVMAFAVKGMKRRTKDN

SEQ ID NO: 73 - protein, 517 aa, Streptococcus agalactiae 2603V/R
  GBS80 Leader/Signal sequence removed
AEVSQERPAKTTVNIYKLQADSYKSEITSNGGIENKDGEVISNYAKLGDNVKGLQGVQFKRYKVKTDISVDELKKLTTVEAADAKVGTILEEGVSLPQKTNAQGLVVDALDSKSNVRYLYVEDLKNSPSNITKAYAVPFVLELPVANSTGTGFLSEINIYPKNVVTDEPKTDKDVKKLGQDDAGYTIGEEFKWFLKSTIPANLGDYEKFEITDKFADGLTYKSVGKIKIGSKTLNRDEHYTIDEPTVDNQNTLKITFKPEKFKEIAELLKGMTLVKNQDALDKATANTDDAAFLEIPVASTINEKAVLGKAIENTFELQYDHTPDKADNPKPSNPPRKPEVHTGGKRFVKKDSTETQTLGGAEFDLLASDGTAVKWTDALIKANTNKNYIAGEAVTGQPIKLKSHTDGTFEIKGLAYAVDANAEGTAVTYKLKETKAPEGYVIPDKEIEFTVSQTSYNTKPTDITVDSADATPDTIKNNKRPSIPNTGGIGTAIFVAIGAAVMAFAVKGMKRRTKDN

SEQ ID NO: 74 - protein, 705 aa, Streptococcus agalactiae 2603V/R
  GBS59 (BP from GBS PI-2a) full length
MKRINKYFAMFSALLLTLTSLLSVAPAFADEATTNTVTLHKILQTESNLNKSNFPGTTGLNGKDYKGGAISDLAGYFGEGSKEIEGAFFALALKEDKSGKVQYVKAKEGNKLTPALINKDGTPEITVNIDEAVSGLTPEGDTGLVFNTKGLKGEFKIVEVKSKSTYNNNGSLLAASKAVPVNITLPLVNEDGVVADAHVYPKNTEEKPEIDKNFAKTNDLTALTDVNRLLTAGANYGNYARDKATATAEIGKVVPYEVKTKIHKGSKYENLVWTDIMSNGLTMGSTVSLKASGTTETFAKDTDYELSIDARGFTLKFTADGLGKLEKAAKTADIEFTLTYSATVNGQAIIDNPESNDIKLSYGNKPGKDLTELPVTPSKGEVTVAKTWSDGIAPDGVNVVYTLKDKDKTVASVSLTKTSKGTIDLGNGIKFEVSGNFSGKFTGLENKSYMISERVSGYGSAINLENGKVTITNTKDSDNPTPLNPTEPKVETHGKKFVKTNEQGDRLAGAQFVVKNSAGKYLALKADQSEGQKTLAAKKIALDEAIAAYNKLSATDQKGEKGITAKELIKTKQADYDAAFIEARTAYEWITDKARAITYTSNDQGQFEVTGLADGTYNLEETLAPAGFAKLAGNIKFVVNQGSYITGGNIDYVANSNQKDATRVENKKVTIPQTGGIGTILFTIIGLSIMLGAVVIMKRRQSKEA

SEQ ID NO: 75 - protein, 675 aa, Streptococcus agalactiae H36B
  GBS59
MKKINKYFAVFSALLLTVTSLFSVAPVFAEEAKTTDTVTLHKIVMPRTAFDGFTAGTKGKDNTDYVGKQIEDLKTYFGSGEAKEIAGAYFAFKNEAGTKYITENGEEVDTLDTTDAKGCAVLKGLTTDNGFKFNTSKLTGTYQIVELKEKSTYNNDGSILADSKAVPVKITLPLVNDNGVVKDAHVYPKNTETKPQVDKNFADKELDYANNKKDKGTVSASVGDVKKYHVGTKILKGSDYKKLIWTDSMTKGLTFNNDIAVTLDGATLDATNYKLVADDQGFRLVLTDKGLEAVAKAAKTKDVEIKITYSATLNGSAVVEVLETNDVKLDYGNNPTIENEPKEGIPVDKKITVNKTWAVDGNEVNKADETVDAVFTLQVKDGDKWVNVDSAKATAATSFKHTFENLDNAKTYRVIERVSGYAPEYVSFVNGVVTIKNNKDSNEPTPINPSEPKVVTYGRKFVKTNKDGKERLAGATFLVKKDGKYLARKSGVATDAEKAAVDSTKSALDAAVKAYNDLTKEKQEGQDGKSALATVSEKQKAYNDAFVKANYSYEWVEDKNAKNVVKLISNDKGQFEITGLTEGQYSLEETQAPTGYAKLSGDVSFNVNATSYSKGSAQDIEYTQGSKTKDAQQVINKKVTIPQTGGIGTIFFTIIGLSIMLGAVVIMKRRQSEEV

SEQ ID NO: 76 - protein, 674 aa, Streptococcus agalactiae 515
  GBS59
MKKINKCLTMFSTLLLILTSLFSVAPAFADDATTDTVTLHKIVMPQAAFDNFTEGTKGKNDSDYVGKQINDLKSYFGSTDAKEIKGAFFVFKNETGTKFITENGKEVDTLEAKDAEGGAVLSGLTKDNGFVFNTAKLKGIYQIVELKEKSNYDNNGSILADSKAVPVKITLPLVNNQGVVKDAHIYPKNTETKPQVDKNFADKDLDYTDNRKDKGVVSATVGDKKEYIVGTKILKGSDYKKLVWTDSMTKGLTFNNNVKVTLDGEDFPVLNYKLVTDDQGFRLALNATGLAAVAAAAKDKDVEIKITYSATVNGSTTVEIPETNDVKLDYGNNPTEESEPQEGTPANQEIKVIKDWAVDGTITDANVAVKAIFTLQEKQTDGTWVNVASHEATKPSRFEHTFTGLDNAKTYRVVERVSGYTPEYVSFKNGVVTIKNNKNSNDPTPINPSEPKVVTYGRKFVKTNQANTERLAGATFLVKKEGKYLARKAGAATAEAKAAVKTAKLALDEAVKAYNDLTKEKQEGQEGKTALATVDQKQKAYNDAFVKANYSYEWVADKKADNVVKLISNAGGQFEITGLDKGTYGLEETQAPAGYATLSGDVNFEVTATSYSKGATTDIAYDKGSVKKDAQQVQNKKVTIPQTGGIGTILFTIIGLSIMLGAVVIMKKRQSEEA

SEQ ID NO: 77 - protein, 693 aa, Streptococcus agalactiae CJB111
  GBS59
MKRINKYFAMFSALLLILTSLLSVAPVFAAEMGNITKTVTLHKIVQTSDNLAKPNFPGINGLNGTKYMGQKLTDISGYFGQGSKEIAGAFFAVMNESQTKYITESGTEVESIDAAGVLKGLTTENGITFNTANLKGTYQIVELLDKSNYKNGDKVLADSKAVPVKITLPLYNEEGIVVDAEVYPKNTEEAPQIDKNFAKANKLLNDSDNSAIAGGADYDKYQAEKAKATAEIGQEIPYEVKTKIQKGSKYKNLAWVDTMSNGLTMGNTVNLEASSGSFVEGTDYNVERDDRGFTLKFTDTGLTKLQKEAETQAVEFTLTYSATVNGAAIDDKPESNDIKLQYGNKPGKKVKEIPVTPSNGEITVSKTWDKGSDLENANVVYTLKDGGTAVASVSLTKTTPNGEINLGNGIKFTVTGAFAGKFSGLTDSKTYMISERIAGYGNTITTGAGSAAITNTPDSDNPTPLNPTEPKVVTHGKKFVKTSSTETERLQGAQFVVKDSAGKYLALKSSATISAQTTAYTNAKTALDAKIAAYNKLSADDQKGTKGETAKAEIKTAQDAYNAAFIVARTAYEWVTNKEDANVVKVTSNADGQFEVSGLATGDYKLEETQAPAGYAKLAGDVDFKVGNSSKADDSGNIDYTASSNKKDAQRIENKKVTIPQTGGIGTILFTIIGLSIMLGAVIIMKRRQSEEA

SEQ ID NO: 78 - protein, 704 aa, Streptococcus agalactiae CJB110
  GBS59
MKKINKYFAVFSALLLTVTSLLSVAPAFADEATTNTVTLHKILQTESNLNKSNFPGTTGLNGDDYKGESISDLAEYFGSGSKEIDGAFFALALEEEKDGVVQYVKAKANDKLTPDLITKGTPATTTKVEEAVGGLTTGTGIVFNTAGLKGNFKIIELKDKSTYNNNGSLLAASKAVPVKITLPLVSKDGVVKDAHVYPKNTETKPEVDKNFAKTNDLTALKDATLLKAGADYKNYSATKATVTAEIGKVIPYEVKTKVLKGSKYEKLVWTDTMSNGLTMGDDVNLAVSGTTTTFIKDIDYTLSIDDRGFTLKFKATGLDKLEEAAKASDVEFTLTYKATVNGQAIIDNPEVNDIKLDYGNKPGTDLSEQPVTPEDGEVKVTKTWAAGANKADAKVVYTLKNATKQVVASVALTAADTKGTINLGKGMTFEITGAFSGTFKGLQNKAYTVSERVAGYTNAINVTGNAVAITNTPDSDNPTPLNPTQPKVETHGKKFVKVGDADARLAGAQFVVKNSAGKFLALKEDAAVSGAQTELATAKTDLDNAIKAYNGLTKAQQEGADGTSAKELINTKQSAYDAAFIKARTAYTWVDEKTKAITFTSNNQGQFEVTGLEVGSYKLEETLAPAGYAKLSGDIEFTVGHDSYTSGDIKYKTDDASNNAQKVFNKKVTIPQTGGIGTILFTIIGLSIMLGAVVIMKRRQSEEA

SEQ ID NO: 79 - protein, 682 aa, Streptococcus agalactiae DK21
  GBS59
MKKINKFFVAFSALLLILTSLLSVAPAFAEEERTTETVTLHKILQTETNLKNSAFPGTKGLDGTEYDGKAIDKLDSYFGNDSKDIGGAYFILANSKGEYIKANDKNKLKPEFSGNTPKTTLNISEAVGGLTEENAGIKFETTGLRGDFQIIELKDKSTYNNGGAILADSKAVPVKITLPLINKDGVVKDAHVYPKNTETKPQIDKNFADKNLDYINNQKDKGTISATVGDVKKYTVGTKILKGSDYKKLVWTDSMTKGLTFNNDVTVTLDGANFEQSNYTLVADDQGFRLVLNATGLSKVAEAAKTKDVEIKINYSATVNGSTVVEKSENNDVKLDYGNNPTTENEPQTGNPVNKEITVRKTWAVDGNEVNKGDEKVDAVFTLQVKDSDKWVNVDSATATAATDFKYTFKNLDNAKTYRVVERVSGYAPAYVSFVGGVVTIKNNKNSNDPTPINPSEPKVVTYGRKFVKTNQDGSERLAGATFLVKNSQSQYLARKSGVATNEAHKAVTDAKVQLDEAVKAYNKLTKEQQESQDGKAALNLIDEKQTAYNEAFAKANYSYEWVVDKNAANVVKLISNTAGKFEITGLNAGEYSLEETQAPTGYAKLSSDVSFKVNDTSYSEGASNDIAYDKDSGKTDAQKVVNKKVTIPQTGGIGTILFTIIGLSIMLGAVVIMKRRQSEEA

SEQ ID NO: 80 - protein, 502 aa, Streptococcus agalactiae COH1
  spb1
MKKKMIQSLLVASLAFGMAVSPVTPIAFAAETGTITVQDTQKGATYKAYKVFDAEIDNANVSDSNKDGASYLIPQGKEAEYKASTDFNSLFTTTTNGGRTYVTKKDTASANEIATWAKSISANTTPVSTVTESNNDGTEVINVSQYGYYYVSSTVNNGAVIMVTSVTPNATIHEKNTDATWGDGGGKTVDQKTYSVGDTVKYTITYKNAVNYHGTEKVYQYVIKDTMPSASVVDLNEGSYEVTITDGSGNITTLTQGSEKATGKYNLLEENNNFTITIPWAATNTPTGNTQNGANDDFFYKGINTITVTYTGVLKSGAKPGSADLPENTNIATINPNTSNDDPGQKVTVRDGQITIKKIDGSTKASLQGAIFVLKNATGQFLNFNDTNNVEWGTEANATEYTTGADGIITITGLKEGTYYLVEKKAPLGYNLLDNSQKVILGDGATDTTNSDNLLVNPTVENNKGTELPSTGGIGTTIFYIIGAILVIGAGIVLVARRRLRS

SEQ ID NO: 81 - protein, 438 aa, Streptococcus agalactiae COH1
  Spb1 Leader/Signal sequence removed
AETGTITVQDTQKGATYKAYKVFDAEIDNANVSDSNKDGASYLIPQGKEAEYKASTDFNSLFTTTTNGGRTYVTKKDTASANEIATWAKSISANTTPVSTVTESNNDGTEVINVSQYGYYYVSSTVNNGAVIMVTSVTPNATIHEKNTDATWGDGGGKTVDQKTYSVGDTVKYTITYKNAVNYHGTEKVYQYVIKDTMPSASVVDLNEGSYEVTITDGSGNITTLTQGSEKATGKYNLLEENNNFTITIPWAATNTPTGNTQNGANDDFFYKGINTITVTYTGVLKSGAKPGSADLPENTNIATINPNTSNDDPGQKVTVRDGQITIKKIDGSTKASLQGAIFVLKNATGQFLNFNDTNNVEWGTEANATEYTTGADGIITITGLKEGTYYLVEKKAPLGYNLLDNSQKVILGDGATDTTNSDNLLVNPTVENNKGTE

SEQ ID NO: 82 - protein, 665 aa, Streptococcus pneumoniae
  RrgB
MKSINKFLTMLAALLLTASSLFSAATVFAAGTTTTSVTVHKLLATDGDMDKIANELETGNYAGNKVGVLPANAKEIAGVMFVWTNTNNEIIDENGQTLGVNIDPQTFKLSGAMPATAMKKLTEAEGAKFNTANLPAAKYKIYEIHSLSTYVGEDGATLTGSKAVPIEIELPLNDVVDAHVYPKNTEAKPKIDKDFKGKANPDTPRVDKDTPVNHQVGDVVEYEIVTKIPALANYATANWSDRMTEGLAFNKGTVKVTVDDVALEAGDYALTEVATGFDLKLTDAGLAKVNDQNAEKTVKITYSATLNDKAIVEVPESNDVTFNYGNNPDHGNTPKPNKPNENGDLTLTKTWVDATGAPIPAGAEATFDLVNAQTGKVVQTVTLTTDKNTVTVNGLDKNTEYKFVERSIKGYSADYQEITTAGEIAVKNWKDENPKPLDPTEPKVVTYGKKFVKVNDKDNRLAGAEFVIANADNAGQYLARKADKVSQEEKQLVVTTKDALDRAVAAYNALTAQQQTQQEKEKVDKAQAAYNAAVIAANNAFEWVADKDNENVVKLVSDAQGRFEITGLLAGTYYLEETKQPAGYALLTSRQKFEVTATSYSATGQGIEYTAGSGKDDATKVVNKKITIPQTGGIGTIIFAVAGAAIMGIAVYAYVKNNKDEDQLA

SEQ ID NO: 83 - protein, 644 aa, Streptococcus pneumoniae
  RrgB
MKSINKFLTMLAALLLTASSLFSAATVFAADNVSTAPDAVTKTLTIHKLLLSEDDLKTWDTNGPKGYDGTQSSLKDLTGVVAEEIPNVYFELQKYNLTDGKEKENLKDDSKWTTVHGGLTTKDGLKIETSTLKGVYRIREDRTKTTYVGPNGQVLTGSKAVPALVTLPLVNNNGTVIDAHVFPKNSYNKPVVDKRIADTLNYNDQNGLSIGTKIPYVVNTTIPSNATFATSFWSDEMTEGLTYNEDVTITLNNVAMDQADYEVTKGNNGFNLKLTEAGLAKINGKDADQKIQITYSATLNSLAVADIPESNDITYHYGNHQDHGNTPKPTKPNNGQITVTKTWDSQPAPEGVKATVQLVNAKTGEKVGAPVELSENNWTYTWSGLDNSIEYKVEEEYNGYSAEYTVESKGKLGVKNWKDNNPAPINPEEPRVKTYGKKFVKVDQKDTRLENAQFVVKKADSNKYIAFKSTAQQAADEKAAATAKQKLDAAVAAYTNAADKQAAQALVDQAQQEYNVAYKEAKFGYVEVAGKDEAMVLTSNTDGQFQISGLAAGTYKLEEIKAPEGFAKIDDVEFVVGAGSWNQGEFNYLKDVQKNDATKVVNKKITIPQTGGIGTIIFAVAGAAIMGIAVYAYVKNNKDEDQLA

SEQ ID NO: 84 - protein, 654 aa, Streptococcus pneumoniae
  RrgB
MKSINKFLTILAALLLTVSSLFSAATVFAAEQKTKTLTVHKLLMTDQELDAWNSDAITTAGYDGSQNFEQFKQLQGVPQGVTEISGVAFELQSYTGPQGKEQENLTNDAVWTAVNKGVTTETGVKFDTEVLQGTYRLVEVRKESTYVGPNGKVLTGMKAVPALITLPLVNQNGVVENAHVYPKNSEDKPTATKTFDTAAGFVDPGEKGLAIGTKVPYIVTTTIPKNSTLATAFWSDEMTEGLDYNGDVVVNYNGQPLDNSHYTLEAGHNGFILKLNEKGLEAINGKDAEATITLKYTATLNALAVADVPEANDVTFHYGNNPGHGNTPKPNKPKNGELTITKTWADAKDAPIAGVEVTFDLVNAQTGEVVKVPGHETGIVLNQTNNWTFTATGLDNNTEYKFVERTIKGYSADYQTITETGKIAVKNWKDENPEPINPEEPRVKTYGKKFVKVDQKDERLKEAQFVVKNEQGKYLALKSAAQQAVNEKAAAEAKQALDAAIAAYTNAADKNAAQAVVDAAQKTYNDNYRAARFGYVEVERKEDALVLTSNTDGQFQISGLAAGSYTLEETKAPEGFAKLGDVKFEVGAGSWNQGDFNYLKDVQKNDATKVVNKKITIPQTGGIGTIIFAVAGAVIMGIAVYAYVKNNKDEDQLA

SEQ ID NO: 85 - protein, 890 aa, Streptococcus agalactiae 2603V/R
  GBS104/SAG0649
MKKRQKIWRGLSVTLLILSQIPFGILVQGETQDTNQALGKVIVKKTGDNATPLGKATFVLKNDNDKSETSHETVEGSGEATFENIKPGDYTLREETAPIGYKKTDKTWKVKVADNGATIIEGMDADKAEKRKEVLNAQYPKSAIYEDTKENYPLVNVEGSKVGEQYKALNPINGKDGRREIAEGWLSKKITGVNDLDKNKYKIELTVEGKTTVETKELNQPLDVVVLLDNSNSMNNERANNSQRALKAGEAVEKLIDKITSNKDNRVALVTYASTIFDGTEATVSKGVADQNGKALNDSVSWDYHKTTFTATTHNYSYLNLTNDANEVNILKSRIPKEAEHINGDRTLYQFGATFTQKALMKANEILETQSSNARKKLIFHVTDGVPTMSYAINFNPYISTSYQNQFNSFLNKIPDRSGILQEDFIINGDDYQIVKGDGESFKLFSDRKVPVTGGTTQAAYRVPQNQLSVMSNEGYAINSGYIYLYWRDYNWVYPFDPKTKKVSATKQIKTHGEPTTLYFNGNIRPKGYDIFTVGIGVNGDPGATPLEAEKFMQSISSKTENYTNVDDTNKIYDELNKYFKTIVEEKHSIVDGNVTDPMGEMIEFQLKNGQSFTHDDYVLVGNDGSQLKNGVALGGPNSDGGILKDVTVTYDKTSQTIKINHLNLGSGQKVVLTYDVRLKDNYISNKFYNTNNRTTLSPKSEKEPNTIRDFPIPKIRDVREFPVLTISNQKKMGEVEFIKVNKDKHSESLLGAKFQLQIEKDFSGYKQFVPEGSDVTTKNDGKIYFKALQDGNYKLYEISSPDGYIEVKTKPVVTFTIQNGEVTNLKADPNANKNQIGYLEGNGKHLITNTPKRPPGVFPKTGGIGTIVYILVGSTFMILTICSFRRKQL

SEQ ID NO: 86 - protein, 901 aa, Streptococcus agalactiae 2603V/R
  GBS67
MRKYQKFSKILTLSLFCLSQIPLNTNVLGESTVPENGAKGKLVVKKTDDQNKPLSKATFVLKTTAHPESKIEKVTAELTGEATFDNLIPGDYTLSEETAPEGYKKTNQTWQVKVESNGKTTIQNSGDKNSTIGQNQEELDKQYPPTGIYEDTKESYKLEHVKGSVPNGKSEAKAVNPYSSEGEHIREIPEGTLSKRISEVGDLAHNKYKIELTVSGKTIVKPVDKQKPLDVVFVLDNSNSMNNDGPNFQRHNKAKKAAEALGTAVKDILGANSDNRVALVTYGSDIFDGRSVDVVKGFKEDDKYYGLQTKFTIQTENYSHKQLTNNAEEIIKRIPTEAPKAKWGSTTNGLTPEQQKEYYLSKVGETFTMKAFMEADDILSQVNRNSQKIIVHVTDGVPTRSYAINNFKLGASYESQFEQMKKNGYLNKSNFLLTDKPEDIKGNGESYFLFPLDSYQTQIISGNLQKLHYLDLNLNYPKGTIYRNGPVKEHGTPTKLYINSLKQKNYDIFNFGIDISGFRQVYNEEYKKNQDGTFQKLKEEAFKLSDGEITELMRSFSSKPEYYTPIVTSADTSNNEILSKIQQQFETILTKENSIVNGTIEDPMGDKINLQLGNGQTLQPSDYTLQGNDGSVMKDGIATGGPNNDGGILKGVKLEYIGNKLYVRGLNLGEGQKVTLTYDVKLDDSFISNKFYDTNGRTTLNPKSEDPNTLRDFPIPKIRDVREYPTITIKNEKKLGEIEFIKVDKDNNKLLLKGATFELQEFNEDYKLYLPIKNNNSKVVTGENGKISYKDLKDGKYQLIEAVSPEDYQKITNKPILTFEVVKGSIKNIIAVNKQISEYHEEGDKHLITNTHIPPKGIIPMTGGKGILSFILIGGAMMSIAGGIYIWKRYKKSSDMSIKKD

SEQ ID NO: 87 - protein, 896 aa, Streptococcus agalactiae H36B
  GBS67
MRKYQKFSKILTLSLFCLSQIPLNTNVLGESTVPENGAKGKLVVKKTDDQNKPLSKATFVLKPTSHSESKVEKVTTEVTGEATFDNLTPGDYTLSEETAPEGYKKTTQTWQVKVESNGKTTIQNSDDKKSIIEQRQEELDKQYPLTGAYEDTKESYNLEHVKNSIPNGKLEAKAVNPYSSEGEHIREIQEGTLSKRISEVNDLDHNKYKIELTVSGKSIIKTINKDEPLDVVFVLDNSNSMKNNGKNNKAKKAGEAVETIIKDVLGANVENRAALVTYGSDIFDGRTVKVIKGFKEDPYYGLETSFTVQTNDYSYKKFTNIAADIIKKIPKEAPEAKWGGTSLGLTPEKKREYDLSKVGETFTMKAFMEADTLLSSIQRKSRKIIVHLTDGVPTRSYAINSFVKGSTYANQFERIKEKGYLDKNNYFITDDPEKIKGNGESYFLFPLDSYQTQIISGNLQKLHYLDLNLNYPKGTIYRNGPVREHGTPTKLYINSLKQKNYDIFNFGIDISGFRQVYNEDYKKNQDGTFQKLKEEAFELSDGEITELMNSFSSKPEYYTPIVTSADVSNNEILSKIQQQFEKILTKENSIVNGTIEDPMGDKINLHLGNGQTLQPSDYTLQGNDGSIMKDSIATGGPNNDGGILKGVKLEYIKNKLYVRGLNLGEGQKVTLTYDVKLDDSFISNKFYDTNGRTTLNPKSEEPDTLRDFPIPKIRDVREYPTITIKNEKKLGEIEFTKVDKDNNKLLLKGATFELQEFNEDYKLYLPIKNNNSKVVTGENGKISYKDLKDGKYQLIEAVSPKDYQKITNKPILTFEVVKGSIQNIIAVNKQISEYHEEGDKHLITNTHIPPKGIIPMTGGKGILSFILIGGAMMSIAGGIYIWKRHKKSSDASIEKD

SEQ ID NO: 88 - protein, 901 aa, Streptococcus agalactiae CJB111
  GBS67
MRKYQKFSKILTLSLFCLSQIPLNTNVLGESTVPENGAKGKLVVKKTDDQNKPLSKATFVLKTTAHPESKIEKVTAELTGEATFDNLIPGDYTLSEETAPEGYKKTNQTWQVKVESNGKTTIQNSGDKNSTIGQNQEELDKQYPPTGIYEDTKESYKLEHVKGSVPNGKSEAKAVNPYSSEGEHIREIPEGTLSKRISEVGDLAHNKYKIELTVSGKTIVKPVDKQKPLDVVFVLDNSNSMNNDGPNFQRHNKAKKAAEALGTAVKDILGANSDNRVALVTYGSDIFDGRSVDVVKGFKEDDKYYGLQTKFTIQTENYSHKQLTNNAEEIIKRIPTEAPKAKWGSTTNGLTPEQQKEYYLSKVGETFTMKAFMEADDILSQVNRNSQKIIVHVTDGVPTRSYAINNFKLGASYESQFEQMKKNGYLNKSNFLLTDKPEDIKGNGESYFLFPLDSYQTQIISGNLQKLHYLDLNLNYPKGTFYRNGPVREHGTPTKLYINSLKQKNYDIFNFGIDISGFRQVYNEDYKKNQDGTFQKLKEEAFELSDGEITELMKSFSSKPEYYTPIVTSSDASNNEILSKIQQQFEKILTKENSIVNGTIEDPMGDKINLQLGNGQTLQPSDYTLQGNDGSIMKDSIATGGPNNDGGILKGVKLEYIKNKLYVRGLNLGEGQKVTLTYDVKLDDSFISNKFYDTNGRTTLNPKSEDPNTLRDFPIPKIRDVREYPTITIKNEKKLGEIEFTKVDKDNNKLLLKGATFELQEFNEDYKLYLPIKNNNSKVVTGENGKISYKDLKDGKYQLIEAVSPKDYQKITNKPILTFEVVKGSIQNIIAVNKQISEYHEEGDKHLITNTHIPPKGIIPMTGGKGILSFILIGGSMMSIAGGIYIWKRYKKSSDISREKD

SEQ ID NO: 89 - protein, 901 aa, Streptococcus agalactiae 515
  GBS67
MRKYQKFSKILTLSLFCLSQIPLNTNVLGESTVPENGAKGKLVVKKTDDQNKPLSKATFVLKTTAHPESKIEKVTAELTGEATFDNLIPGDYTLSEETAPEGYKKTNQTWQVKVESNGKTTIQNSGDKNSTIGQNQEELDKQYPPTGIYEDTKESYKLEHVKGSVPNGKSEAKAVNPYSSEGEHIREIPEGTLSKRISEVGDLAHNKYKIELTVSGKTIVKPVDKQKPLDVVFVLDNSNSMNNDGPNFQRHNKAKKAAEALGTAVKDILGANSDNRVALVTYGSDIFDGRSVDVVKGFKEDDKYYGLQTKFTIQTENYSHKQLTNNAEEIIKRIPTEAPKAKWGSTTNGLTPEQQKEYYLSKVGETFTMKAFMEADDILSQVNRNSQKIIVHVTDGVPTRSYAINNFKLGASYESQFEQMKKNGYLNKSNFLLTDKPDDIKGNGESYFLFPLDSYQTQIISGNLQKLHYLDLNLNYPKGTIYRNGPVKEHGTPTKLYINSLKQKNYDIFNFGIDISGFRQVYNEEYKKNQDGTFQKLKEEAFKLSDGEITELMRSFSSKPEYYTPIVTSADTSNNEILSKIQQQFETILTKENSIVNGTIEDPMGDKINLQLGNGQILQPSDYTLQGNDGSVMKDGIATGGPNNDGGILKGVKLEYIGNKLYVRGLNLGEGQKVTLTYDVKLDDSFISNKFYDTNGRTTLNPKSEDPNTLRDFPIPKIRDVREYPTITIKNEKKLGEIEFIKVDKDNNKLLLKGATFELQEFNEDYKLYLPIKNNNSKVVTGENGKISYKDLKDGKYQLIEAVSPEDYQKITNKPILTFEVVKGSIKNIIAVNKQISEYHEEGDKHLITNTHIPPKGIIPKTGGKGILSFILIGGAMMSIAGGIYIWKRYKKSSDMSIKKD

SEQ ID NO: 90 - protein, 901 aa, Streptococcus agalactiae NEM316
  GBS67
MRKYQKFSKILTLSLFCLSQIPLNTNVLGESTVPENGAKGKLVVKKTDDQNKPLSKATFVLKTTAHPESKIEKVTAELTGEATFDNLIPGDYTLSEETAPEGYKKTNQTWQVKVESNGKTTIQNSGDKNSTIGQNHEELDKQYPPTGIYEDTKESYKLEHVKGSVPNGKSEAKAVNPYSSEGEHIREIPEGTLSKRISEVGDLAHNKYKIELTVSGKTIVKPVDKQKPLDVVFVLDNSNSMNNDGPNFQRHNKAKKAAEALGTAVKDILGANSDNRVALVTYGSDIFDGRSVDVVKGFKEDDKYYGLQTKFTIQTENYSHKQLTNNAEEIIKRIPTEAPRAKWGSTTNGLTPEQQKQYYLSKVGETFTMKAFMEADDILSQVDRNSQKIIVHITDGVPTRSYAINNFKLGASYESQFEQMKKNGYLNKSNFLLTDKPEDIKGNGESYFLFPLDSYQTQIISGNLQKLHYLDLNLNYPKGTIYRNGPVREHGTPTKLYINSLKQKNYDIFNFGIDISAFRQVYNEDYKKNQDGTFQKLKEEAFELSDGEITELMKSFSSKPEYYTPIVTSSDASNNEILSKIQQQFEKVLTKENSIVNGTIEDPMGDKINLQLGNGQTLQPSDYTLQGNDGSIMKDSIATGGPNNDGGILKGVKLEYIKNKLYVRGLNLGEGQKVTLTYDVKLDDSFISNKFYDTNGRTTLNPKSEDPNTLRDFPIPKIRDVREYPTITIKNEKKLGEIEFTKVDKDNNKLLLKGATFELQEFNEDYKLYLPIKNNNSKVVTGENGKISYKDLKDGKYQLIEAVSPKDYQKITNKPILTFEVVKGSIQNIIAVNKQISEYHEEGDKHLITNTHIPPKGIIPMTGGKGILSFILIGGSMMSIAGGIYIWKRYKKSSDISREKD

SEQ ID NO: 91 - protein, 896 aa, Streptococcus agalactiae DK21
  GBS67
MRKYQKFSKILTLSLFCLSQIPLNTNVLGESTVPENGAKGKLVVKKTDDQNKPLSKATFVLKPTSHSESKVEKVTTEVTGEATFDNLTPGDYTLSEETAPEGYKKTTQTWQVKVESNGKTTIQNSDDKKSIIEQRQEELDKQYPLTGAYEDTKESYNLEHVKNSIPNGKLEAKAVNPYSSEGEHIREIQEGTLSKRISEVNDLDHNKYKIELTVSGKSIIKTINKDEPLDVVFVLDNSNSMKNNGKNNKAKKAGEAVETIIKDVLGANVENRAALVTYGSDIFDGRTVKVIKGFKEDPYYGLETSFTVQTNDYSYKKFTNIAADIIKKIPKEAPEAKWGGTSLGLTPEKKREYDLSKVGETFTMKAFMEADTLLSSIQRKSRKIIVHLTDGVPTRSYAINSFVTGSTYANQFERIKEKGYLDKNNYFITDDPEKIKGNGESYFLFPLDSYQTQIISGNLQKLHYLDLNLNYPKGTIYRNGPVREHGTPTKLYINSLKQKNYDIFNFGIDISGFRQVYNEDYKKNQDGTFQKLKEEAFELSDGEITELMNSFSSKPEYYTPIVTSADVSNNEILSKIQQQFEKILTKENSIVNGTIEDPMGDKINLQLGNGQTLQPSDYTLQGNDGSIMKDSIATGGPNNDGGILKGVKLEYIKNKLYVRGLNLGEGQKVTLTYDVKLDDSFISNKFYDTNGRTTLNPKSEEPDTLRDFPIPKIRDVREYPTITIKNEKKLGEIEFTKVDKDNNKLLLKGATFELQEFNEDYKLYLPIKNNNSKVVTGENGKISYKDLKDGKYQLIEAVSPKDYQKITNKPILTFEVVKGSIQNIIAVNKQISEYHEEGDKHLITNTHIPPKGIIPMTGGKGILSFILIGGAMMSIAGGIYIWKRHKKSSDASIEKD

SEQ ID NO: 92 - protein, 896 aa, Streptococcus agalactiae CJB110
  GBS67
MRKYQKFSKILTLSLFCLSQIPLNTNVLGESTVPENGAKGKLVVKKTDDQNKPLSKATFVLKTTAHPESKIEKVTAEVTGEATFDNLTPGDYTLSEETAPEGYKKTTQTWQVKVESNGKTTIQNSDDKKSIIEQRQEELDKQYPLTGAYEDTKESYNLEHVKNSIPNGKLEAKAVNPYSSEGEHIREIQEGTLSKRISEVNDLDHNKYKIELTVSGKSIIKTINKDEPLDVVFVLDNSNSMKNNGKNNKAKKAGEAVETIIKDVLGANVENRAALVTYGSDIFDGRTVKVIKGFKEDPYHGLETSFTVQTNDYSYKKFTNIAADIIKKIPKEAPEAKWGGTSLGLTPEKKREYDLSKVGETFTMKAFMEADTLLSSIQRKSRKIIVHLTDGVPTRSYAINSFVTGSTYANQFERIKEKGYLDKNNYFITDDPEKIKGNGESYFLFPLDSYQTQIISGNLQKLHYLDLNLNYPKGTIYRNGPVREHGTPTKLYINSLKQKNYDIFNFGIDISGFRQVYNEDYKKNQDGTFQKLKEEAFELSGGEITELMKSFSSKPEYYTPIVTSADVSNNEILSKIQQQFEKILTKENSIVNGTIEDPMGDKINLQLGNGQTLQPSDYTLQGNDGSIMKDSIATGGPNNDGGILKGVKLEYIKNKLYVRGLNLGEGQKVTLTYDVKLDDSFISNKFYDTNGRTTLNPKSEEPDTLRDFPIPKIRDVREYPTITIKNEKKLGEIEFTKVDKDNNKLLLKGATFELQEFNEDYKLYLPIKNNNSKVVTGENGKISYKDLKDGKYQLIEAVSPKDYQKITNKPILTFEVVKGSIQNIIAVNKQISEYHEEGDKHLITNTHIPPKGIIPMTGGKGILSFILIGGAMMSIAGGIYIWKKHKKSSDASIEKD

SEQ ID NO: 93 - protein, 1434 aa, Streptococcus agalactiae COH1
  GBS1524/SAN1519
MLKKCQTFIIESLKKKKHPKEWKIIMWSLMILTTFLTTYFLILPAITVEETKTDDVGITLENKNSSQVTSSTSSSQSSVEQSKPQTPASSVTETSSSEEAAYREEPLMFRGADYTVTVTLTKEAKIPKNADLKVTELKDNSATFKDYKKKALTEVAKQDSEIKNFKLYDITIESNGKEAEPQAPVKVEVNYDKPLEASDENLKVVHFKDDGQTEVLKSKDTAETKNTSSDVAFKTDSFSIYAIVQEDNTEVPRLTYHFQNNDGTDYDFLTASGMQVHHQIIKDGESLGEVGIPTIKAGEHFNGWYTYDPTTGKYGDPVKFGEPITVTETKEICVRPFMSKVATVTLYDDSAGKSILERYQVPLDSSGNGTADLSSFKVSPPTSTLLFVGWSKTQNGAPLSESEIQALPVSSDISLYPVFKESYGVEFNTGDLSTGVTYIAPRRVLTGQPASTIKPNDPTRPGYTFAGWYTAASGGAAFDFNQVLTKDTTLYAHWSPAQTTYTINYWQQSATDNKNATDAQKTYEYAGQVTRSGLSLSNQTLTQQDINDKLPTGFKVNNTRTETSVMIKDDGSSVVNVYYDRKLITIKFAKYGGYSLPEYYYSYNWSSDADTYTGLYGTTLAANGYQWKTGAWGYLANVGNNQVGTYGMSYLGEFILPNDTVDSDVIKLFPKGNIVQTYRFFKQGLDGTYSLADTGGGAGADEFTFTEKYLGFNVKYYQRLYPDNYLFDQYASQTSAGVKVPISDEYYDRYGAYHKDYLNLVVWYERNSYKIKYLDPLDNTELPNFPVKDVLYEQNLSSYAPDTTTVQPKPSRPGYVWDGKWYKDQAQTQVFDFNTTMPPHDVKVYAGWQKVTYRVNIDPNGGRLSKTDDTYLDLHYGDRIPDYTDITRDYIQDPSGTYYYKYDSRDKDPDSTKDAYYTTDTSLSNVDTTTKYKYVKDAYKLVGWYYVNPDGSIRPYNFSGAVTQDINLRAIWRKAGDYHIIYSNDAVGTDGKPALDASGQQLQTSNEPTDPDSYDDGSHSALLRRPTMPDGYRFRGWWYNGKIYNPYDSIDIDAHLADANKNITIKPVIIPVGDIKLEDTSIKYNGNGGTRVENGNVVTQVETPRMELNSTTTIPENQYFTRTGYNLIGWHHDKDLADTGRVEFTAGQSIGIDNNPDATNTLYAVWQPKEYTVRVSKTVVGLDEDKTKDFLFNPSETLQQENFPLRDGQTKEFKVPYGTSISIDEQAYDEFKVSESITEKNLATGEADKTYDATGLQSLTVSGDVDISFTNTRIKQKVRLQKVNVENDNNFLAGAVFDIYESDANGNKASHPMYSGLVTNDKGLLLVDANNYLSLPVGKYYLTETKAPPGYLLPKNDISVLVISTGVTFEQNGNNATPIKENLVDGSTVYTFKITNSKGTELPSTGGIGTHIYILVGLALALPSGLILYYRKKI

SEQ ID NO: 94 - protein, 886 aa, Streptococcus pneumoniae
  RrgA
MKKVRKIFQKAVAGLCCISQLTAFSSIVALAETPETSPAIGKVVIKETGEGGALLGDAVFELKNNTDGTTVSQRTEAQTGEAIFSNIKPGTYTLTEAQPPVGYKPSTKQWTVEVEKNGRTTVQGEQVENREEALSDQYPQTGTYPDVQTPYQIIKVDGSEKNGQHKALNPNPYERVIPEGTLSKRIYQVNNLDDNQYGIELTVSGKTVYEQKDKSVPLDVVILLDNSNSMSNIRNKNARRAERAGEATRSLIDKITSDPENRVALVTYASTIFDGTEFTVEKGVADKNGKRLNDSLFWNYDQTSFTTNTKDYSYLKLTNDKNDIVELKNKVPTEAEDHDGNRLMYQFGATFTQKALMKADEILTQQARQNSQKVIFHITDGVPTMSYPINFNHATFAPSYQNQLNAFFSKSPNKDGILLSDFITQATSGEHTIVRGDGQSYQMFTDKTVYEKGAPAAFPVKPEKYSEMKAAGYAVIGDPINGGYIWLNWRESILAYPFNSNTAKITNHGDPTRWYYNGNIAPDGYDVFTVGIGINGDPGTDEATATSFMQSISSKPENYTNVTDTTKILEQLNRYFHTIVTEKKSIENGTITDPMGELIDLQLGTDGRFDPADYTLTANDGSRLENGQAVGGPQNDGGLLKNAKVLYDTTEKRIRVTGLYLGTDEKVTLTYNVRLNDEFVSNKFYDTNGRTTLHPKEVEQNTVRDFPIPKIRDVRKYPEITISKEKKLGDIEFIKVNKNDKKPLRDAVFSLQKQHPDYPDIYGAIDQNGTYQNVRTGEDGKLTFKNLSDGKYRLFENSEPAGYKPVQNKPIVAFQIVNGEVRDVTSIVPQDIPAGYEFTNDKHYITNEPIPPKREYPRTGGIGMLPFYLIGCMMMGGVLLYTRKHP

SEQ ID NO: 95 - protein, 307 aa, Streptococcus agalactiae 2603V/R
  GBS052/SAG0646
MKQTLKLMFSFLLMLGTMFGISQTVLAQETHQLTIVHLEARDIDRPNPQLEIAPKEGTPIEGVLYQLYQLKSTEDGDLLAHWNSLTITELKKQAQQVFEATTNQQGKATFNQLPDGIYYGLAVKAGEKNRNVSAFLVDLSEDKVIYPKIIWSTGELDLLKVGVDGDTKKPLAGVVFELYEKNGRTPIRVKNGVHSQDIDAAKHLETDSSGHIRISGLIHGDYVLKEIETQSGYQIGQAETAVTIEKSKTVTVTIENKKVPTPKVPSRGGLIPKTGEQQAMALVIIGGILIALALRLLSKHRKHQNKD

SEQ ID NO: 96 - protein, 308 aa, Streptococcus agalactiae 2603V/R
  GBS150/SAG1404
MKKIRKSLGLLLCCFLGLVQLAFFSVASVNADTPNQLTITQIGLQPNTTEEGISYRLWTVTDNLKVDLLSQMTDSELNQKYKSILTSPTDTNGQTKIALPNGSYFGRAYKADQSVSTIVPFYIELPDDKLSNQLQINPKRKVETGRLKLIKYTKEGKIKKRLSGVIFVLYDNQNQPVRFKNGRFTTDQDGITSLVTDDKGEIEVEGLLPGKYIFREAKALTGYRISMKDAVVAVVANKTQEVEVENEKETPPPTNPKPSQPLFPQSFLPKTGMIIGGGLTILGCIILGILFIFLRKTKNSKSERNDTV

SEQ ID NO: 97 - protein, 401 aa, Streptococcus pneumoniae
  RrgC
MTMQKMQKMISRIFFVMALCFSLVWGAHAVQAQEDHTLVLQLENYQEVVSQLPSRDGHRLQVWKLDDSYSYDDRVQIVRDLHSWDENKLSSFKKTSFEMTFLENQIEVSHIPNGLYYVRSIIQTDAVSYPAEFLFEMTDQTVEPLVIVAKKTDTMTTKVKLIKVDQDHNRLEGVGFKLVSVARDGSEKEVPLIGEYRYSSSGQVGRTLYTDKNGEIFVTNLPLGNYRFKEVEPLAGYAVTTLDTDVQLVDHQLVTITVVNQKLPRGNVDFMKVDGRTNTSLQGAMFKVMKEESGHYTPVLQNGKEVVVTSGKDGRFRVEGLEYGTYYLWELQAPTGYVQLTSPVSFTIGKDTRKELVTVVKNNKRPRIDVPDTGEETLYILMLVAILLFGSGYYLTKKPNN

SEQ ID NO: 98 - protein, 255 aa, Streptococcus agalactiae
  SrtC1 lacking N-terminal transmembrane domain
HQSRAIMDYQDRVTHMDENDYKKIINRAKEYNKQFKTSGMKWHMTSQERLDYNSQLAIDKTGNMGYISIPKINIKLPLYHGTSEKVLQTSIGHLEGSSLPIGGDSTHSILSGHRGLPSSRLFSDLDKLKVGDHWTVSILNETYTYQVDQIRTVKPDDLRDLQIVKGKDYQTLVTCTPYGVNTHRLLVRGHRVPNDNGNALVVAEAIQIEPIYIAPFIAIFLTLILLLISLEVTRRARQRKKILKQAMRKEENNDL

SEQ ID NO: 99 - protein, 203 aa, Streptococcus agalactiae; identical to SEQ ID NO: 23
  SrtC1

SEQ ID NO: 100 - protein, 254 aa, Streptococcus agalactiae
  srtC1
QSRAIMDYQDRVTHMDENDYKKIINRAKEYNKQFKTSGMKWHMTSQERLDYNSQLAIDKTGNMGYISIPKINIKLPLYHGTSEKVLQTSIGHLEGSSLPIGGDSTHSILSGHRGLPSSRLFSDLDKLKVGDHWTVSILNETYTYQVDQIRTVKPDDLRDLQIVKGKDYQTLVTCTPYGVNTHRLLVRGHRVPNDNGNALVVAEAIQIEPIYIAPFIAIFLTLILLLISLEVTRRARQRKKILKQAMRKEENNDL

SEQ ID NO: 101 - protein, 197 aa, Streptococcus agalactiae
  SrtC1
QSRAIMDYQDRVTHMDENDYKKIINRAKEYNKQFKTSGMKWHMTSQERLDYNSQLAIDKTGNMGYISIPKINIKLPLYHGTSEKVLQTSIGHLEGSSLPIGGDSTHSILSGHRGLPSSRLFSDLDKLKVGDHWTVSILNETYTYQVDQIRTVKPDDLRDLQIVKGKDYQTLVTCTPYGVNTHRLLVRGHRVPNDNGN

SEQ ID NO: 102 - protein, 254 aa, Streptococcus agalactiae
  SrtC1
QSRAIMDYQDRVTHMDENDYKKIINRAKEYNKQFKTSGMKAHMTSQERLDYNSQLAIDKTGNMGYISIPKINIKLPLYHGTSEKVLQTSIGHLEGSSLPIGGDSTHSILSGHRGLPSSRLFSDLDKLKVGDHWTVSILNETYTYQVDQIRTVKPDDLRDLQIVKGKDYQTLVTCTPYGVNTHRLLVRGHRVPNDNGNALVVAEAIQIEPIYIAPFIAIFLTLILLLISLEVTRRARQRKKILKQAMRKEENNDL

SEQ ID NO: 103 - protein, 254 aa, Streptococcus agalactiae
  SrtC1 C188A mutant
QSRAIMDYQDRVTHMDENDYKKIINRAKEYNKQFKTSGMKWHMTSQERLDYNSQLAIDKTGNMGYISIPKINIKLPLYHGTSEKVLQTSIGHLEGSSLPIGGDSTHSILSGHRGLPSSRLFSDLDKLKVGDHWTVSILNETYTYQVDQIRTVKPDDLRDLQIVKGKDYQTLVTATPYGVNTHRLLVRGHRVPNDNGNALVVAEAIQIEPIYIAPFIAIFLTLILLLISLEVTRRARQRKKILKQAMRKEENNDL

SEQ ID NO: 104 - DNA, 807 bp, Streptococcus agalactiae
  source 1..807: /organism=“Streptococcus agalactiae” /note=“SrtC1” /mol_type=“unassigned DNA”
atggcttatccttcacttgctaattattggaattcatttcaccaatctcgagcgattatggattaccaagaccgcgtaacgcatatggatgaaaacgattataaaaaaattattaaccgagccaaagaatataataagcaatttaaaacttcaggaatgaagtggcacatgactagccaagagcgtttggattataattcacaactggctatcgataaaacgggtaatatgggttatatttcaattccaaagataaacataaaattaccactttatcatggtacaagtgaaaaagtgcttcaaacttctattggtcatttagaaggaagtagtcttccaattggaggagactcaactcattctattttatcaggacatagaggtttaccctcttcaaggcttttttctgatttggataagttaaaagttggagaccactggacagtcagtatcttaaatgaaacatatacttatcaagtggatcaaatcagaacagttaaaccggatgatttgagggatttacaaattgttaaaggtaaagactaccaaactttggtgacgtgtacaccatatggcgttaatacccatcggttactagtgagaggacatcgtgtaccaaacgataatggtaacgctttggtagtagcagaggcaatacaaatagagcctatttatatcgcaccatttatcgctattttccttactttgattttacttttaatctctttagaagtaactaggagagcacgtcaacgtaagaaaattttaaaacaagcaatgagaaaggaagagaacaatgatttataa

SEQ ID NO: 105 - protein, 199 aa, Streptococcus agalactiae
  SrtC2
MIRRYSANFLAILGIILVSSGIYWGWYNINQAHQADLTSQHIVKVLDKSITHQVKGSENGELPVKKLDKTDYLGTLDIPNLKLHLPVAANYSFEQLSKTPTRYYGSYLTNNMVICAHNFPYHFDALKNVDMGTDVYFTTTTGQIYHYKISNREIIEPTAIEKVYKTATSDNDWDLSLFTCTKAGVARVLVRCQLIDVKN

SEQ ID NO: 106 - protein, 199 aa, Streptococcus agalactiae
  SrtC2 Cys substituted with Ala
MIRRYSANFLAILGIILVSSGIYWGWYNINQAHQADLTSQHIVKVLDKSITHQVKGSENGELPVKKLDKTDYLGTLDIPNLKLHLPVAANYSFEQLSKTPTRYYGSYLTNNMVIAAHNFPYHFDALKNVDMGTDVYFTTTTGQIYHYKISNREIIEPTAIEKVYKTATSDNDWDLSLFTATKAGVARVLVRAQLIDVKN

SEQ ID NO: 107 - DNA, 600 bp, Streptococcus agalactiae
  source 1..600: /organism=“Streptococcus agalactiae” /note=“SrtC2” /mol_type=“unassigned DNA”
gtgattagaagatattcagcaaattttttagctatactcggaattattctggtaagttctggaatctattggggttggtataatattaatcaggcgcatcaagctgatttaacttctcagcatattgtcaaggtgcttgataaatctattacgcatcaagtaaagggttcagaaaatggagaattacctgtaaaaaagttggataaaacagattacttgggaactctggatattccgaacttaaaactgcatttaccggtagctgctaattatagttttgaacaactgtctaagacgcctacaaggtattatggttcttatttaactaataacatggtgatttgtgcgcataattttccttatcattttgatgctttaaaaaatgtagatatgggaacggatgtttattttacaactacaacagggcaaatctatcactacaaaatcagtaatagagaaattattgaaccaacagcgattgaaaaagtttataaaactgccacatcagacaatgattgggacttaagcttgtttacttgtacaaaggcaggagtagctagagtattagtgcgctgtcaattaattgatgttaaaaattaa

SEQ ID NO: 108 - protein, 169 aa, Streptococcus agalactiae
  SrtC2 No signal peptide
QAHQADLTSQHIVKVLDKSITHQVKGSENGELPVKKLDKTDYLGTLDIPNLKLHLPVAANYSFEQLSKTPTRYYGSYLTNNMVICAHNFPYHFDALKNVDMGTDVYFTTTTGQIYHYKISNREIIEPTAIEKVYKTATSDNDWDLSLFTCTKAGVARVLVRCQLIDVKN

SEQ ID NO: 109 - protein, 190 aa, Streptococcus agalactiae
  SrtC2 no N-terminal domain
LAILGIILVSSGIYWGWYNINQAHQADLTSQHIVKVLDKSITHQVKGSENGELPVKKLDKTDYLGTLDIPNLKLHLPVAANYSFEQLSKTPTRYYGSYLTNNMVICAHNFPYHFDALKNVDMGTDVYFTTTTGQIYHYKISNREIIEPTAIEKVYKTATSDNDWDLSLFTCTKAGVARVLVRCQLIDVKN

SEQ ID NO: 110 - protein, 502 aa, Streptococcus agalactiae; identical to SEQ ID NO: 80
  GBS1523/SAN1518 Full length

SEQ ID NO: 111 - protein, 438 aa, Streptococcus agalactiae; identical to SEQ ID NO: 81
  GBS1523/SAN1518 Q41K mutation

SEQ ID NO: 112 - protein, 438 aa, Streptococcus agalactiae
  GBS1523/SAN1518 No signal sequence Q41K mutation
AETGTITVQDTKKGATYKAYKVFDAEIDNANVSDSNKDGASYLIPQGKEAEYKASTDFNSLFTTTTNGGRTYVTKKDTASANEIATWAKSISANTTPVSTVTESNNDGTEVINVSQYGYYYVSSTVNNGAVIMVTSVTPNATIHEKNTDATWGDGGGKTVDQKTYSVGDTVKYTITYKNAVNYHGTEKVYQYVIKDTMPSASVVDLNEGSYEVTITDGSGNITTLTQGSEKATGKYNLLEENNNFTITIPWAATNTPTGNTQNGANDDFFYKGINTITVTYTGVLKSGAKPGSADLPENTNIATINPNTSNDDPGQKVTVRDGQITIKKIDGSTKASLQGAIFVLKNATGQFLNFNDTNNVEWGTEANATEYTTGADGIITITGLKEGTYYLVEKKAPLGYNLLDNSQKVILGDGATDTTNSDNLLVNPTVENNKGTE

SEQ ID NO: 113 - DNA, 48 bp, Artificial Sequence
  source 1..48: /organism=“Artificial Sequence” /note=“GFP-LPxTG Forward PCR Primer” /mol_type=“unassigned DNA”
attccacaaacaggtggtattggtacataacgcgacttaattaaacgg

SEQ ID NO: 114 - DNA, 48 bp, Artificial Sequence
  source 1..48: /organism=“Artificial Sequence” /note=“GFP-LPxTG Reverse PCR Primer” /mol_type=“unassigned DNA”
tgtaccaataccacctgtttgtggaatcttgtacagctcgtccatgcc

SEQ ID NO: 115 - DNA, 880 bp, Artificial Sequence
  source 1..880: /organism=“Artificial Sequence” /note=“EGFP DNA sequence” /mol_type=“unassigned DNA”
ctttaagaaggagatatacatacccatgggatctgataaaattcatcatcatcatcatcacgaaaacctgtacttccagggcatggtgagcaagggcgaggagctgttcaccggggtggtgcccatcctggtcgagctggacggcgacgtaaacggccacaagttcagcgtgtccggcgagggcgagggcgatgccacctacggcaagctgaccctgaagttcatctgcaccaccggcaagctgcccgtgccctggcccaccctcgtgaccaccctgacctacggcgtgcagtgcttcagccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcatggccgacaagcagaagaacggcatcaaggtgaacttcaagatccgccacaacatcgaggacggcagcgtgcagctcgccgaccactaccagcagaacacccccatcggcgacggccccgtgctgctgcccgacaaccactacctgagcacccagtccgccctgagcaaagaccccaacgagaagcgcgatcacatggtcctgctggagttcgtgaccgccgccgggatcactctcggcatggacgagctgtacaagtaacgcgacttaattaaacggtctccagcttggctgttttggcggatgagagaagattttcagcctgatacagattaaatc

SEQ ID NO: 116 - protein, 258 aa, Artificial Sequence
  EGFP Amino Acid Sequence
MGSDKIHHHHHHENLYFQGMVSKGEELFTGVVPILVELDGDVNGHKFSVSGEGEGDATYGKLTLKFICTTGKLPVPWPTLVTTLTYGVQCFSRYPDHMKQHDFFKSAMPEGYVQERTIFFKDDGNYKTRAEVKFEGDTLVNRIELKGIDFKEDGNILGHKLEYNYNSHNVYIMADKQKNGIKVNFKIRHNIEDGSVQLADHYQQNTPIGDGPVLLPDNHYLSTQSALSKDPNEKRDHMVLLEFVTAAGITLGMDELYK

SEQ ID NO: 117 - DNA, 907 bp, Artificial Sequence
  source 1..907: /organism=“Artificial Sequence” /note=“GFP comprising LPxTG tag” /mol_type=“unassigned DNA”
ctttaagaaggagatatacatacccatgggatctgataaaattcatcatcatcatcatcacgaaaacctgtacttccagggcatggtgagcaagggcgaggagctgttcaccggggtggtgcccatcctggtcgagctggacggcgacgtaaacggccacaagttcagcgtgtccggcgagggcgagggcgatgccacctacggcaagctgaccctgaagttcatctgcaccaccggcaagctgcccgtgccctggcccaccctcgtgaccaccctgacctacggcgtgcagtgcttcagccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcatggccgacaagcagaagaacggcatcaaggtgaacttcaagatccgccacaacatcgaggacggcagcgtgcagctcgccgaccactaccagcagaacacccccatcggcgacggccccgtgctgctgcccgacaaccactacctgagcacccagtccgccctgagcaaagaccccaacgagaagcgcgatcacatggtcctgctggagttcgtgaccgccgccgggatcactctcggcatggacgagctgtacaagattccacaaacaggtggtattggtacataacgcgacttaattaaacggtctccagcttggctgttttggcggatgagagaagattttcagcctgatacagattaaatc

SEQ ID NO: 118 - protein, 267 aa, Artificial Sequence
  GFP comprising LPxTG sequence tag
MGSDKIHHHHHHENLYFQGMVSKGEELFTGVVPILVELDGDVNGHKFSVSGEGEGDATYGKLTLKFICTTGKLPVPWPTLVTTLTYGVQCFSRYPDHMKQHDFFKSAMPEGYVQERTIFFKDDGNYKTRAEVKFEGDTLVNRIELKGIDFKEDGNILGHKLEYNYNSHNVYIMADKQKNGIKVNFKIRHNIEDGSVQLADHYQQNTPIGDGPVLLPDNHYLSTQSALSKDPNEKRDHMVLLEFVTAAGITLGMDELYKIPQTGGIGT

(19) United States
(12) Patent Application Publication
(10) Pub. No.: US 2015/0273042 A1
(43) Pub. Date: Oct. 1, 2015

(54) PILUS PROTEINS AND COMPOSITIONS

(71) Applicant: NOVARTIS AG, Basel (CH)

(72) Inventors: Domenico Maione, Siena (IT); Immaculada Margarit Y Ros, Siena (IT); Roberta Cozzi, Siena (IT); Cira Daniela Rinaudo, Castelnuovo Berardenga (IT); Maddalena Lazzarin, Terrassa Padovana (IT); Francesca Zerbini, Siena (IT)

(21) Appl. No.: 14/380,260

(22) PCT Filed: Feb. 23, 2013
(86) PCT No.: PCT/EP2013/053644

§ 371 Date: Aug. 21, 2014

Related U.S. Application Data
(60) Provisional application No. 61/602,827, filed on Feb. 24, 2012.
(60) Provisional application No. 61/704,866, filed on Sep. 24, 2012.

(51) Int. Cl. A61K 39/09; C12N 9/52; C07K 14/315

(52) U.S. Cl. CPC A61K 39/092; C07K 14/315; C12N 9/52; C12Y 304/22

(57) ABSTRACT

The invention provides methods of forming pili in vitro and proteins suitable for use in these methods. The invention also provides pili produced by these methods and compositions comprising these pili for the treatment and prevention of bacterial disease, in particular of conditions caused by Streptococcus.

### TECHNICAL FIELD

The invention provides methods of forming pili in vitro and mutant sortase enzymes and proteins suitable for use in these methods. The invention also provides pili produced by these methods and compositions comprising these pili for the treatment and prevention of bacterial disease, in particular of conditions caused by Streptococcus. The invention also provides general methods of ligating proteins and sortase enzymes for use in same.

### BACKGROUND ART

Most bacterial pathogens comprise pili (also known as fimbrae), long filamentous structures extending from their surface, that are often responsible for initial adhesion of bacteria to tissues during host colonization. Gram-negative bacteria have been known for many years to have pili, typically formed by non-covalent interactions between pilin subunits. More recently, Gram-positive bacteria, including Streptococcus bacteria, have also been shown to have pili typically formed through covalent association of subunits by sortases that are encoded by pilus-specific pathogenicity islands.

The Gram-positive bacterium Streptococcus agalactiae (or “group B streptococcus”, abbreviated to “GBS”), for example, has three pilus variants, each encoded by a distinct pathogenicity island, PI-1, PI-2a or PI-2b [1, 2]. Each pathogenicity island consists of: i) genes encoding the three structural components of the pilus (the pilus backbone protein (BP) and 2 ancillary proteins (AP1 and AP2)); and ii) genes encoding 2 sortase proteins (SrtC1 and SrtC2) that are involved in the assembly of the pilus. All GBS strains carry at least one of these 3 pathogenicity islands.

Similar pathogenicity islands are present in other Gram-positive bacteria including Streptococcus pyogenes or “group A streptococcus”, abbreviated to “GAS”), and Streptococcus pneumoniae (also known as pneumococcus). The pathogenicity island in pneumococcus encodes the 3 structural components of the pilus (RrgA, RrgB and RrgC) and three sortases (SrtC1, SrtC2 and SrtC3) which catalyse pilus formation. In GAS, the FCT regions encode the backbone and accessory proteins and polymerisation of these proteins is also mediated by a sortase (SACT).

Pilus structures in these Gram-positive bacteria are considered to be interesting vaccine candidates and work has been done on assessing the immunogenicity of purified recombinant proteins from pilus structures. It is also desirable to study these proteins in their native form within assembled pili but currently, the only way to do this is by the laborious process of purifying wild-type pili from the bacteria. One object of the invention is therefore to provide a process for producing recombinant pili in vitro without the need to purify wild-type pili.

The streptococcal bacteria discussed above are associated with serious disease. GBS causes bacteremia and meningitis in immunocompromised individuals and in neonates. GAS is a frequent human pathogen, estimated to be present in between 5-15% of normal individuals without signs of disease. When host defences are compromised or when GAS is introduced to vulnerable tissues or hosts, however, an acute infection occurs. Diseases caused by GAS include puerperal fever, scarlet fever, erysipelas, pharyngitis, impetigo, necrotising fasciitis, myositis and streptococcal toxic shock syndrome. Pneumococcus is the most common cause of acute bacterial meningitis in adults and in children over 5 years of age

Investigations have been conducted into the development of protein-based vaccines against these Streptococcal bacteria but currently, no protein-based vaccines are commercially available. There therefore remains a need for effective vaccines against Streptococcal infection. It is a further object of the invention to provide immunogenic compositions which can be used in the development of vaccines against streptococcal infection.

### SUMMARY OF THE INVENTION

In a first aspect the invention provides a method of ligating at least two moieties comprising contacting the at least two moieties with a pilus-related sortase C enzyme in vitro under conditions suitable for a sortase mediated transpeptidation reaction to occur, wherein the pilus-related sortase C enzyme comprises an exposed active site.

Particularly the pilus-related sortase C enzyme is from Streptococcus, more particularly from Streptococcus agalactiae (GBS), Streptococcus pneumonia (pneumococcus) and Streptococcus pyogenes (GAS). Yet more particularly the pilus-related sortase C enzyme is a sortase C1 enzyme (srtC1), sortase C2 enzyme (SrtC2) or a sortase C3 enzyme (SrtC3).

In certain embodiments the pilus-related sortase C enzyme mutation comprises a deletion of part or all of the lid. Particularly the mutation comprises a deletion of the amino acids at positions 84, 85 and/or 86 of the amino acid sequence of the GBS sortase C1 enzyme of PI-2a (SEQ ID NO:3), or the deletion of amino acids at corresponding positions in the amino acid sequence of another pilus-related sortase C enzyme.

In other embodiments the mutation comprises substitution of the amino acids at positions 84, 85 and/or 86 of the amino acid sequence of the GBS sortase C1 enzyme of PI-2a (SEQ ID NO:3), or the substitution of amino acids at corresponding positions in the amino acid sequence of another sortase C enzyme.

Particularly the pilus-related sortase C enzyme comprises or consists of an amino acid sequence selected from the group consisting of SEQ ID NO: 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70 and 71.

In one embodiment of the invention, the method is a method of forming a recombinant or artificial pilus in vitro. This, the at least two moieties comprise an LPxTG motif and a pilin motif. For example, the pilin motif may comprise the amino acids YPAN. ‘X’ in any sortase recognition motif disclosed herein may be any standard or non-standard amino acid and every variation is disclosed. In some embodiments, X is selected from the 20 standard amino acids found most commonly in proteins found in living organisms. Where the recognition motif is LPXTG or LPXT, X may be D, E, A, N, Q, K, or R. In particular, X is selected from K, S, E, L, A, N in an LPXTG or LPXT motif.

Particularly the at least two moieties are from Gram-positive bacteria. The at least two moieties may be from the same strain or type of Gram-positive bacteria or from different strains or types of Gram positive bacteria. Yet more particularly, the at least two moieties are Streptococcal polypeptides. Still yet more particularly, the at least two moieties are Streptococcal backbone proteins and/or ancillary proteins.

For example, the at least two moieties comprise or consist of an amino acid sequence: (a) having 50% or more identity (e.g. 60%, 65%, 70%, 75%, 80%, 85%, 90%, 91%, 92%, 93%, 94%, 95%, 96%, 97%, 98%, 99%, 99.5% or more) to a polypeptide having the amino acid sequence of any one of SEQ ID NOs: 72, 73, 74, 75, 76, 77, 78, 79, 80, 81, 82, 83, 84, 85, 86, 87, 88, 89, 90, 91, 92, 93, 94, 95, 96, or 97; or (b) that is a fragment of at least ‘n’ consecutive amino acids of one of these sequences wherein ‘n’ is 20 or more (e.g. 25, 30, 35, 40, 50, 60, 70, 80, 90, 100, 150 or more; e.g. 20 or more; or e.g. 50 or more; or e.g. 80 or more).

In other aspects of the invention, there is provided an artificial or recombinant pilus obtained or obtainable from the aforementioned method. In one embodiment there is provided an artificial or recombinant pilus which comprises at least two variants of backbone protein GBS59. Particularly the at least two variants are selected from Group B Streptococcus strains 2603, H36B, 515, CJB111, CJB110 and DK21. Yet more particularly, the artificial or recombinant pilus is a chimeric pilus comprising at least one variant of GBS backbone protein GBS59 selected from Streptococcus strains 2603, H36B, 515, CJB111, CJB110 and DK21 and at least one backbone protein from Streptococcus pneumonia selected from the group consisting of RrgA, RrgB and RrgC. In other embodiments artificial or recombinant pili further comprise GBS80 and/or GBS1523.

In particular aspects of the invention, the artificial or recombinant pilus is for use in medicine, yet more particularly for use in preventing or treating Streptococcal infection. Thus, in another embodiment there is provided a method of treating or preventing Streptococcal infection in a patient in need thereof comprising administering an effective amount of an artificial or recombinant pilus formed by the methods of the invention to a patient.

In a second aspect of the invention, there is provided a method wherein the at least two moieties comprise a first moiety comprising the amino acid motif LPXTG, wherein X is any amino acid, and a second moiety comprising at least one amino acid.

Particularly the first moiety is a first polypeptide and the second moiety is a second polypeptide. In certain embodiments, the first polypeptide and the second polypeptide are from Gram-positive bacteria. For example, the first polypeptide and the second polypeptide may be from the same type or strain of Gram-positive bacteria or from different types or strains of Gram positive bacteria. In some embodiments, the first polypeptide and the second polypeptide are Streptococcal polypeptides. For example, the first polypeptide and the second polypeptide may be Streptococcal backbone proteins and/or ancillary proteins.

In some embodiments of the invention, either the first moiety or the second moiety comprises a detectable label. By way of non-limiting example, the detectable label may be a fluorescent label, a radiolabel, a chemiluminescent label, a phosphorescent label, a biotin label, or a streptavidin label. In some embodiments, the first moiety or the second moiety may be a polypeptide and the other moiety may be a protein or glycoprotein on the surface of a cell. In yet further embodiments, either the first moiety or the second moiety is a polypeptide and the other moiety comprises amino acids conjugated to a solid support. In still yet further embodiments, either the first moiety or the second moiety is a polypeptide and the other moiety comprises at least one amino acid conjugated to a polynucleotide.

The method of the invention may be used to ligate the N-terminus of a first moiety to the N-terminus of a second moiety. The method of the invention may be used to ligate the C-terminus of a first moiety to the C-terminus of a second moiety. Alternatively, the first moiety and the second moiety are the N-terminus and C-terminus of a moiety such as a polypeptide chain, and ligation results in the formation of a circular polypeptide. Thus, there is provided conjugate obtained or obtainable from the method described herein.

In other aspects of the invention, there is provided a kit comprising a sortase C1 or a sortase C2 enzyme from Streptococcus agalactiae and a moiety comprising the amino acid motif LPXTG, wherein X is any amino acid.

In another aspect of the invention, there is provided a sortase C enzyme from Streptococcus comprising a mutation in its lid region, particularly a sortase C enzyme from Streptococcus which is from Streptococcus agalactiae (GBS), Streptococcus pneumonia (pneumococcus) or Streptococcus pyogenes (GAS). Yet more particularly a sortase C enzyme from Streptococcus wherein the sortase C enzyme from Streptococcus is a sortase C1 enzyme, sortase C2 enzyme or a sortase C3 enzyme. In certain embodiments, there is provided a sortase C enzyme from Streptococcus wherein the mutation comprises deletion of part or all of the lid region of the sortase C enzyme. Particularly the mutation comprises deletion of the amino acids at positions 84, 85 and/or 86 of the amino acid sequence of the GBS sortase C1 enzyme of PI-2a (SEQ ID NO:3), or the deletion of amino acids at corresponding positions in the amino acid sequence of another sortase C enzyme. In other embodiments there is provided a sortase C enzyme from Streptococcus wherein the mutation comprises substitution of the amino acids at positions 84, 85 and/or 86 of the amino acid sequence of the GBS sortase C1 enzyme of PI-2a (SEQ ID NO:3), or the substitution of amino acids at corresponding positions in the amino acid sequence of another sortase C enzyme

Particularly there is provided a sortase C enzyme from Streptococcus which comprises a mutation in its lid region and wherein the sortase C enzyme comprises or consists of an amino acid sequence selected from SEQ ID NO: 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70 or 71.

### BRIEF DESCRIPTION OF DRAWINGS

FIG. 1: Alignment of GBS sortase C sequences showing location of the lid region in bold and underlined.

FIG. 2: Alignment of Streptococcus pneumoniae and Streptococcus pyogenes (GAS) sortase C sequences showing location of the lid region in bold and underlined.

FIG. 3: A: Conserved amino acid motifs identified in the backbone protein of GBS pilus 2a (BP-2a), GBS59 (strain 515, TIGR annotation SAL_{—}1486). Pilin motif: containing a highly conserved lysine residue (Lys189); E-box: containing a highly conserved glutamic acid residue (Glu589); Sorting signal: containing residues IPQTGG located at positions 641-646. B: Immunoblot performed with an antibody recognising the backbone protein of GBS pilus 2a (α-BP), showing that Lys189 of the pilin motif of BP-2a is required for pilus polymerization by wild type sortase C. A plasmid was generated encoding a mutant BP-2a carrying a substitution at Lys189 with Ala (BP_{K189A}). A GBS mutant strain lacking backbone proteins (GBS_{ΔBP}) was transformed with this plasmid (lane 2), or a control plasmid encoding wild-type BP-2a (BP_{WT}) (lane 1). The star indicates the location of the protein bands corresponding to the monomeric, unpolymerised BP-2a protein. High molecular weight protein bands, corresponding to polymerised BP-2a, are detectable only in cell extracts of GBS transformed with the plasmid encoding wild-type BP-2a (lane 1). C: Immunoblots performed with antibodies recognising the backbone protein of GBS pilus 2a (a-BP) (lanes 1, 2 and 3) or ancillary protein of GBS pilus 2a (a-AP1) (lanes 4 and 5), showing that the IPQTG motif of BP-2a is required for pilus polymerization. A plasmid was generated encoding a mutant BP-2a carrying a deletion of the IPQTG sorting signal (BP_{ΔIPQTG}). A GBS mutant strain lacking backbone proteins (GBS_{ΔBP}) was transformed with this plasmid (lanes 3 and 4). As controls, a control plasmid encoding wild-type BP-2a (BP_{WT}) was used (lane 1), or no plasmid (ΔBP) (lanes 2 and 5). The star indicates the location of the protein bands corresponding to the monomeric, unpolymerised BP-2a protein. The triangle indicates the protein band corresponding to monomeric AP1 protein. The box indicates the protein band corresponding to BP-2a-AP1 conjugates. High molecular weight protein bands, corresponding to polymerised BP-2a, are detectable only in cell extracts of GBS transformed with the plasmid encoding wild-type BP-2a.

FIG. 4: A: Protein gel showing that wild-type GBS sortase fails to catalyse in vitro polymerization of wild-type backbone protein. Various concentrations of recombinant backbone protein (BP) (25, 100 and 200 μM) were incubated at 37° C. with wild-type sortase C1 of PI-2a (SrtC1_{WT}) for 0, 24 and 48 hours. The proteins contained in the reaction mixture were resolved by sodium-dodecylsulfate polyacrylamide gel electrophoresis (SDS-PAGE) and visualised. No formation of high molecular weight bands, corresponding to polymerized BP, was detectable. The star indicates monomeric BP. The hash indicates SrtC1_{WT}. Lane 1: BP 25 μM+SrtC1_{WT} t0, Lane 2: BP 25 μM+SrtC1_{WT} t24h, Lane 3: BP 25 μM+SrtC1_{WT} t48h; Lane 4: BP 100 μM+SrtC1_{WT} t0, Lane 5: BP 100 μM+SrtC1_{WT} t24, Lane 6: BP 100 μM+SrtC1_{WT} t48h; Lane 7: BP 200 μM+SrtC1_{WT} t0, Lane 8: BP 200 μM+SrtC1_{WT} t24. B: Protein gel showing that wild-type backbone protein (BP) can form BP-BP homodimers in the absence of catalytic sortase activity, explaining the additional bands observed in panel A. Various concentrations of recombinant BP (25 and 100 μM) were incubated for 0, 24, 48 and 72 hours and the proteins contained in the reaction mixture were visualised by SDS-PAGE. Lane 1: BP 25 μM t0h, Lane 2: BP 25 μM t24h, Lane 3: BP 25 μM t48h, Lane 4: BP 25 μM t72; Lane 5: BP 100 μM t0h, Lane 6: BP 100 μM t24h, Lane 7: BP 100 μM t48h, Lane 8: BP 100 μM t72h.

FIG. 5: A: Protein gel showing that a mutant GBS sortase carrying a mutation in the lid region is able to catalyse in vitro polymerization of wild-type backbone protein (BP). Various concentrations of recombinant BP (100 and 200 μM) were incubated with mutant sortase C1 of PI-2a carrying a tyrosine to alanine substitution at position 86 (SrtC1_{Y86A}) for 0, 24 or 48 hours and the proteins contained in the reaction mixture visualised by SDS-PAGE. The star indicates monomeric BP. High molecular weight bands (≧260 kDa), corresponding to polymerized BP, were detectable after 24 or 48 hours of incubation. Lane 1: BP 100 μM+SrtC1_{Y86A} t0h, Lane 2: BP 100 μM+SrtC1_{Y86A} t24h, Lane 3: BP 100 μM+SrtC1_{Y86A} t48h; Lane 4: BP 200 μM+SrtC1_{Y86A} t0h, Lane 5: BP 200 μM+SrtC1_{Y86A} t24h B: Immunoblot performed with an antibody recognising the backbone protein of GBS pilus 2a (αBP), showing that the pattern of polymerized BP is similar to BP polymers contained in pili from wild-type bacteria (here GBS strain 515). The star indicates monomeric BP. Lane 1: BP, Lane 2: SrtC1_{Y86A}, Lane 3: BP+SrtC1_{Y86A}, Lane 4: GBS515 Wild Type Pili. C: Protein gel showing the effect of different concentrations of SrtC1_{Y86A} on the efficiency of BP polymerisation. 10, 50 or 100 μM of SrtC1_{Y86A} were mixed with BP and incubated for 0 hours, 48 hours, 3 and 4 days and the proteins contained in the reaction mixtures were visualised by SDS-PAGE. The star indicates monomeric BP. D: Protein gel showing the effect of different concentrations of BP on the efficiency of BP polymerisation. 25, 50 or 100 μM of BP were mixed with 25 μM of SrtC1_{Y86A} and incubated for 0 hours, 3 days, 5 days and 7 days and the proteins contained in the reaction mixtures were visualised by SDS-PAGE. The star indicates monomeric BP.

FIG. 6: Protein gel showing that in vitro polymerised pili structures can be successfully purified. 25 μM of SrtC1_{Y86A} were incubated with 100 μM of BP-2a at 37° C. for 7 days. The proteins contained within the mixture were separated into fractions by size exclusion chromatography and visualised by SDS-PAGE. The high-molecular weight fractions containing purified polymerised BP elute first (white box), followed by monomeric BP (star) and SrtC1_{Y86A} (cross).

FIG. 7: Protein gel showing that mutant sortase enzymes polymerize pilus proteins from a variety of gram positive bacteria. A: 25 μM of SrtC1_{Y86A} (GBS sortase C1 of PI-2a) were incubated with 100 μM of backbone protein PI-1 of GBS (also referred to as GBS 80) at 37° C. for 7 days and the proteins contained in the reaction mixtures were visualised by SDS-PAGE. As controls, SrtC1_{Y86A} or GBS 80 alone were incubated under the same conditions. The star indicates monomeric BP. Lane 1: SrtC1_{Y86A}, Lane 2: BP PI-1, Lane 3: SrtC1_{Y86A}+BP PI-1. B: 25 μM of SrtC1_{Y86A} (GBS sortase C1 of PI-2a) were incubated with 50 or 100 μM of pilus protein from Streptococcus pneumoniae (also referred to as RrgB) at 37° C. for 3 days and the proteins contained in the reaction mixtures were visualised by SDS-PAGE. As controls, SrtC1_{Y86A} or RrgB alone were incubated under the same conditions. The star indicates monomeric RrgB. Lane 1: SrtC1_{Y86A}, Lane 2: RrgB, Lane 3: SrtC1_{Y86A}+RrgB (50 μM), Lane 4: SrtC1_{Y86A}+RrgB (100 μM).

FIG. 8: Pairwise sequence alignment of homologous SrtC1 sortases from PI-2a of GBS strain 515 and PI-2b of GBS strain A909. The catalytic triad (single underline) is conserved, while the canonical lid motif (double underline) is not present in PI-2b SrtC1. Instead there is a tryptophan that appears to mimic the lid function.

FIG. 9: Pairwise alignment of SrtC2 sortase from PI-2b (SAK_{—}1437) and SrtC1 sortase from PI-2a (SAL_{—}1484). SrtC2 lacks the lid sequence (highlighted in box), and the C terminal trans-membrane domain. Three cysteine residues are present in PI-2b SrtC2 sequence (marked with crosses).

FIG. 10: Western blot of total protein extracts from culture of a mutant strain derived from GBS 515 in which the PI-2a island has been deleted (515Δ2a) and from the wild type A909 strain complemented by a plasmid containing SrtC1 and BP genes or BP gene alone. Antibodies against BP were used. High-molecular weight signals indicate pili polymerization in the complemented strains. M: Marker; Lane 1: 515Δ2a; Lane 2: 515Δ2a+BP; Lane 3: 515Δ2a+BP+SrtC1; Lane 4: 515Δ2a+BP+SrtC1; Lane 5: A909+BP; Lane 6: A909+BP+SrtC1.

FIG. 11: SDS-PAGE of polymerization reactions. Lane 1: SrtC1Y86A+BP-2a-515; Lane 2: SrtC1Y86A+BP-2a-H36B; Lane 3: SrtC1Y86A+BP-2a-CJB111; Lane 4: Marker; Lane 5: SrtC1Y86A+BP-2a-515-H36B-CJB111.

FIG. 12A: Western blot with polyclonal antibody against BP-1. Lane 1: SrtC1Y86A; Lane 2: BP-2a-515 variant; Lane 3: BP-2a-H36B variant; Lane 4: BP-1; Lane 5: RrgB; Lane 6: SrtC1Y86A+BP-1; Lane 7: SrtC1Y86A+BP-2a-515+BP-1; Lane 8: SrtC1Y86A+BP-2a-H36B+BP-1; Lane 9: SrtC1Y86A+RrgB; Lane 10: SrtC1Y86A+BP-2a-515+RrgB; Lane 11: SrtC1Y86A+BP-2a-H36B+RrgB.

FIG. 12B: Western blot with polyclonal antibody against RrgB. Lane 1: SrtC1Y86A; Lane 2: BP-2a-515 variant; Lane 3: BP-2a-H36B variant; Lane 4: BP-1; Lane 5: RrgB; Lane 6: SrtC1Y86A+BP-1; Lane 7: SrtC1Y86A+BP-2a-515+BP-1; Lane 8: SrtC1Y86A+BP-2a-H36B+BP-1; Lane 9: SrtC1Y86A+RrgB; Lane 10: SrtC1Y86A+BP-2a-515+RrgB; Lane 11: SrtC1Y86A+BP-2a-H36B+RrgB.

FIG. 13: Mutant SrtC can polymerize Green Fluorescent Protein (GFP) tagged with an IPQTG sequence.

FIG. 14A: The LPXTG motif is essential for in vitro pilus polymerization. Progression of the reaction between the SrtC1Y86A and recombinant BP-2a ΔIPQTG at T0, 48 and 72 hours of incubation at 37° C. The concentrations of both SrtC1Y86A and BP-2a ΔIPQTG were fixed at 25 μM and 100 μM respectively. No formation of high molecular weight pattern could be identify, showing that the LPXTG like-motif is necessary for the BP polymerization. As controls the SrtC1Y86A (on the left) and BP-2a ΔIPQTG (on the right) were incubated alone.

FIG. 14B: The lysine of pilin motif is not essential for in vitro pilus polymerization. The SrtC1Y86A (25 μM) and the recombinant BP-2a K189A (100 μM) were mixed at 37° C. and at different time points (0, 48h and 72h) the reactions were analysed by SDS-PEGE. A patter of high molecular weight could be identified, showing that the SrtC1Y86A used another nucleophile different from the lysine189.

FIG. 14C: When SrtC1Y86A was mixed with recombinant forms of the ancillary proteins (AP1-2a and AP2-2a), that in vivo can be polymerized only in the presence of the BP-2a protein (data not shown), some HMW structures were formed. These data demonstrate that SrtC1Y86A can use different nucleophile/s to resolve the acyl-intermediate between the enzyme and the LPXTG-like sorting signal.

### DETAILED DESCRIPTION OF THE INVENTION

Structural studies of pilus-related C-sortases in gram positive bacteria have demonstrated that the active site of many of these enzymes contains a catalytic triad of amino acids that are covered by a mobile “lid” region in the absence of substrate. Thus, a feature of pilus-related sortases is the presence of a lid that not only blocks active site access, i.e. it encapsulates the active site, but also carries two key residues, generally an Asp and a hydrophobic amino acid, that interact within the catalytic cleft itself, serving as ‘anchors’. Generally sequences corresponding to lid regions can be identified in all pilus-related sortases characterized to date. In particular, this lid structure has been demonstrated to be present in the sortase C1 enzymes from GBS PI-1, PI-2a and PI-2b [3], in the sortase C1, sortase C2 and sortase C3 enzymes from Streptococcus pneumoniae [4, 5], and in the sortase C1 enzyme from GAS. Mutation of the lid region in the sortase C1 enzyme from GBS PI-2a has been shown not to have an adverse impact on pilus production in complementation studies [3] but until now, no studies have been conducted into the ability of mutant sortases to polymerise proteins in vitro.

The inventors have now found that sortase C enzymes are capable of polymerising proteins in vitro more effectively than wild-type sortase C enzyme, for example, resulting in the production of recombinant pili. Wild type sortase C enzyme comprise a “mobile lid” region encapsulating the active site in a closed conformation in the absence of substrate. For example, the lid of SrtC1 harbors 3 residues, Asp84, Pro85, and Tyr86 which make interactions with residues of the active site and surroundings. Thus, sortase C enzymes are inactive in vitro and unable to ligate or polymerise moieties such as pilin backbone and ancillary proteins. The inventors have now discovered that by mutating the lid region, the catalytic site can be exposed rendering these mutated enzymes active in vitro. As discussed below, and surprisingly, these mutated enzymes are more active than their wild-type counterparts and yet more surprisingly are capable of recognising a broader range of amino acids. Particularly, mutated enzymes of the invention possess or comprise an exposed catalytic site which is not encapsulated by a “lid” and is available to catalyze a transpeptidation reaction to form an acyl enzyme intermediate in vitro.

The methods of the invention can thus be used to produce artificial or recombinant pili without the need for the labourious purification procedures currently used. Surprisingly, these mutant sortase C enzymes can also be used to polymerise proteins from a variety of sources such as gram positive bacteria, not just proteins derived from the same bacteria as the mutant sortase C enzyme itself. Furthermore, the pili resulting from these methods are immunogenic and may be used in the development of vaccines to treat or prevent diseases caused by the gram positive bacteria from which the component proteins of the pili are derived.

Some pilin subunits within the pilus contain intra-protein isopeptide bonds that form spontaneously, presumably stabilizing the structure of the pilus. Thus, in the context of vaccines, immunisation of a subject with proteins in the form of an artificial or recombinant pilus structure mimicking those encountered by the immune system during invasion/infection may also have advantages in terms of the presence of additional epitopes, such as structural or conformational epitopes based on three-dimensional structure. Such structural or conformational epitopes may be absent from subunit vaccines when the pilus proteins are provided in compositions comprising the isolated, purified forms or as conjugates, such as glycoconjugates. Thus, the polymerised pili proteins may comprise three-dimensional epitopes not predictable from the structure of the proteins alone.

Mutant Sortase C Enzymes

The mutant sortase C enzyme used in the methods of the invention is derived from a wild-type sortase C enzyme from Streptococcus. The mutant sortase C enzyme may, for example, be derived from a wild-type sortase C enzyme from Streptococcus agalactiae (GBS), Streptococcus pneumonia (pneumococcus) or Streptococcus pyogenes (GAS). The mutant sortase C enzyme may be derived from a sortase C1 enzyme, a sortase C2 enzyme or a sortase C3 enzyme. The mutant sortase C enzyme is derived from a wild-type streptococcal sortase C enzyme that comprises a lid region. The lid region is the structural loop of about 15-18 amino acids that covers the catalytic triad of amino acids found in the active site of a sortase C enzyme in the absence of a substrate. The lid region is located within the soluble core domain of the sortase C enzyme, between the signal peptide and transmembrane (TM) region located at the N-terminal of the enzyme and the positively charged domain located at the C-terminal of the enzyme. The location of the lid region in a variety of wild-type Streptococcal sortase C enzymes is summarised in the table below. These sequences are all wild-type sequences which include the N-terminal signal peptide.

TABLE 1
Location of lid region in Streptococcal sortases

| | Sequence of | Location of | Location of signal |
|---|---|---|---|
| Sortase | wild-type sortase | lid region | peptide and TM region |
| GBS sortase C1 of PI-1 | SEQ ID NO: 1 | Amino acids 86-102 | Amino acids 1-41 |
| GBS sortase C2 of PI-1 | SEQ ID NO: 2 | Amino acids 79-95 | Amino acids 1-41 |
| GBS sortase C1 of PI-2a | SEQ ID NO: 3 | Amino acids 81-96 | Amino acids 1-42 |
| GBS sortase C2 of PI-2a | SEQ ID NO: 4 | Amino acids 84-99 | Amino acids 1-46 |
| GBS sortase C1 of PI-2b | SEQ ID NO: 5 | Amino acids 49-65 | Amino acids 1-13 |
| Pneumococcus sortase C1 | SEQ ID NO: 6 | Amino acids 52-70 | Amino acids 1-16 |
| Pneumococcus sortase C2 | SEQ ID NO: 7 | Amino acids 45-62 | Amino acids 1-8 |
| Pneumococcus sortase C3 | SEQ ID NO: 8 | Amino acids 70-81 | Amino acids 1-31 |
| GAS sortase C1 | SEQ ID NO: 9 | Amino acids 40-57 | Amino acids 1-4 |

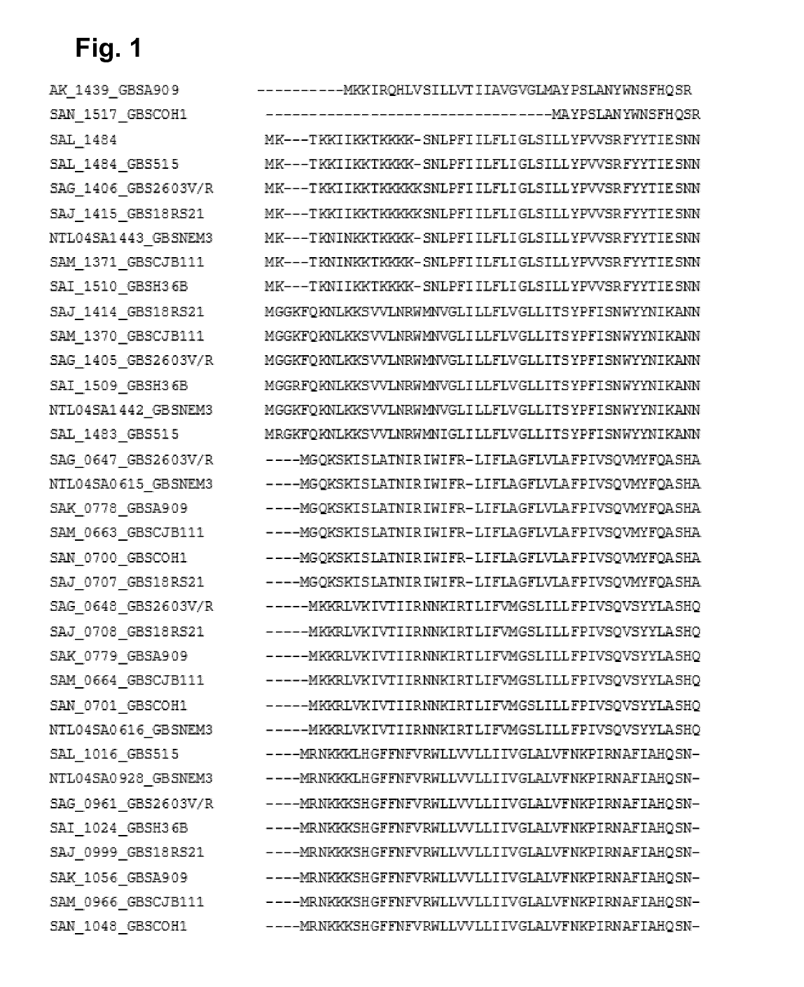

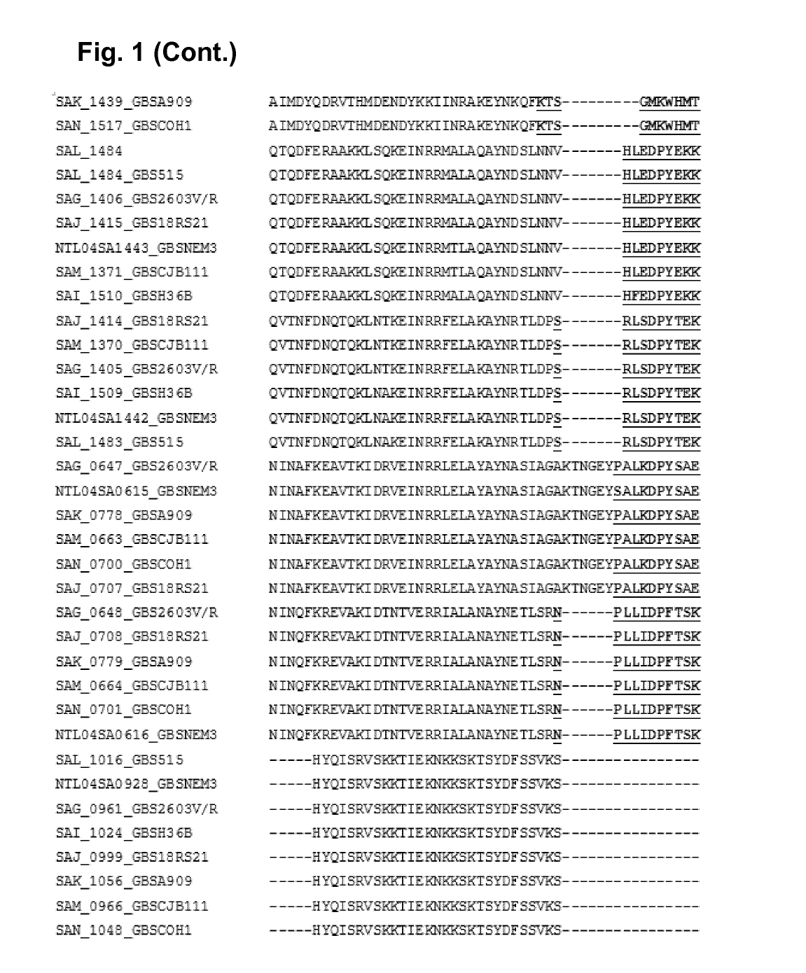

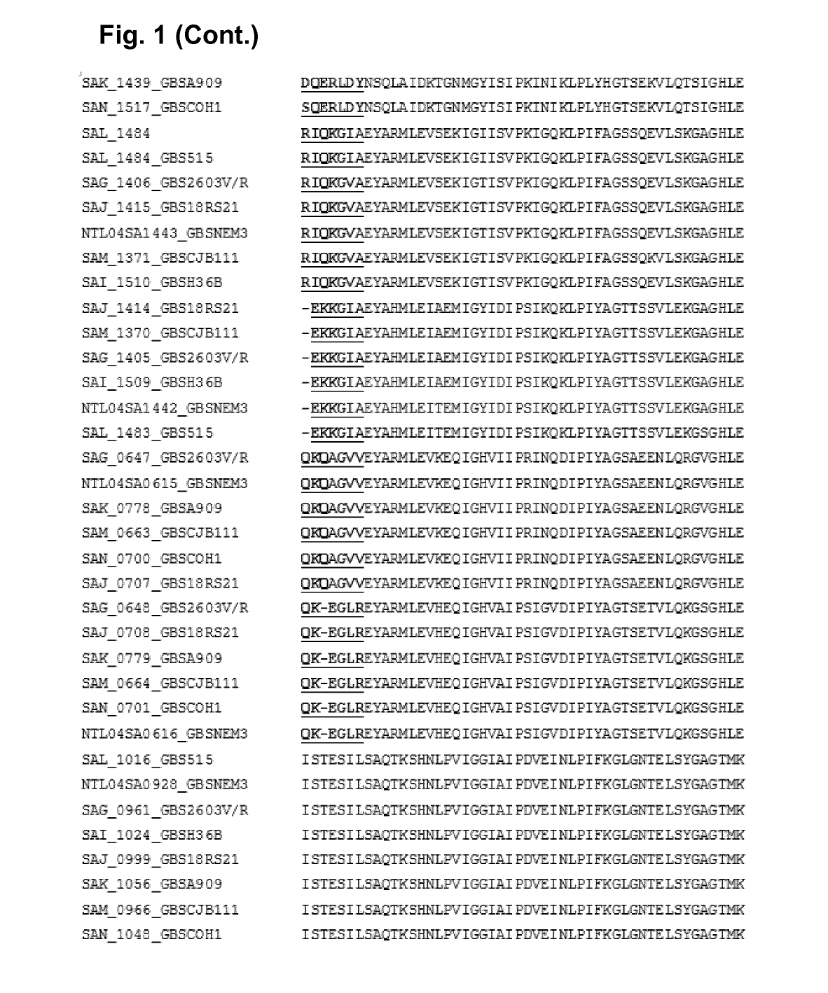

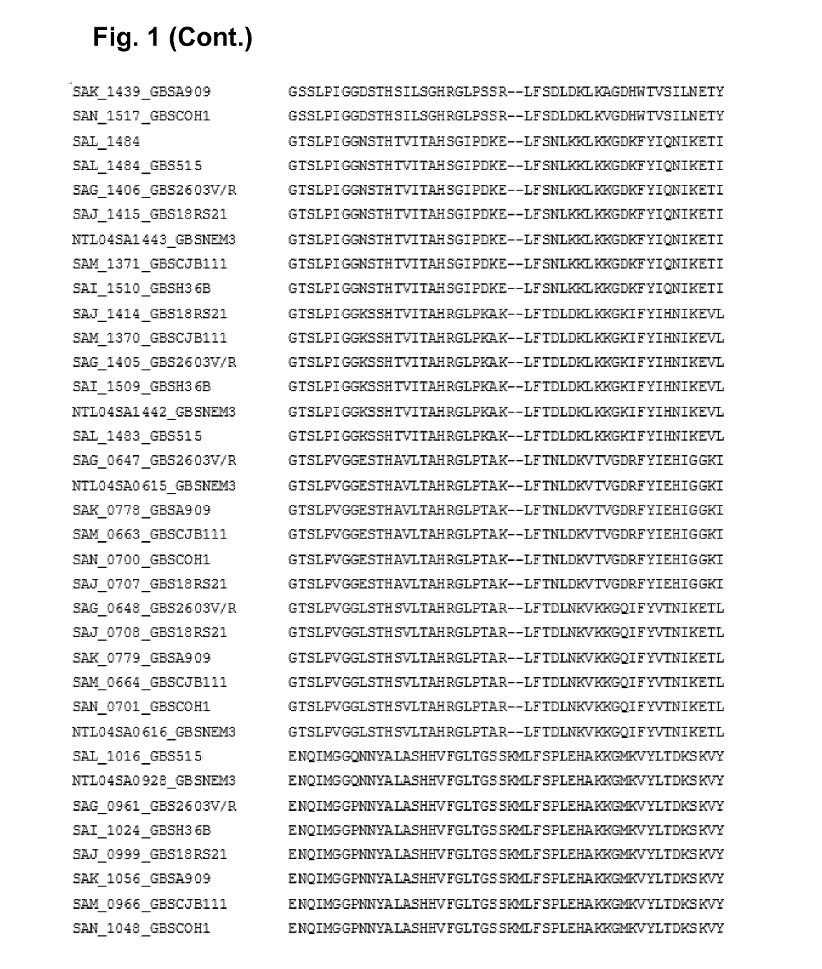

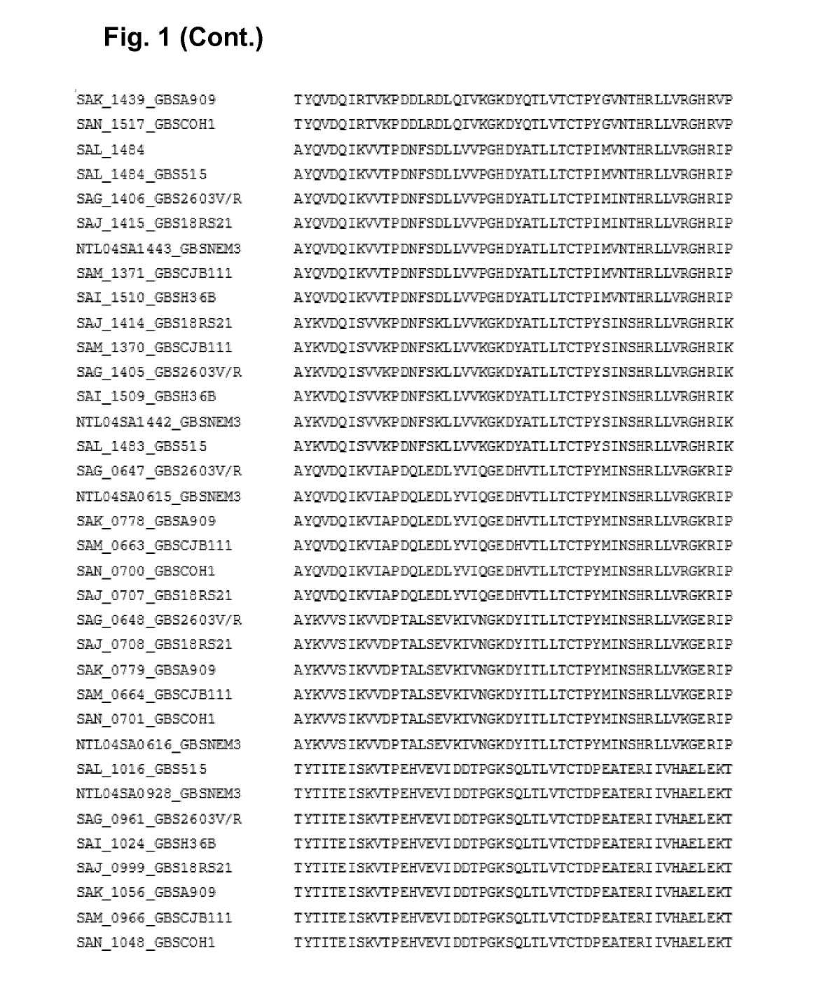

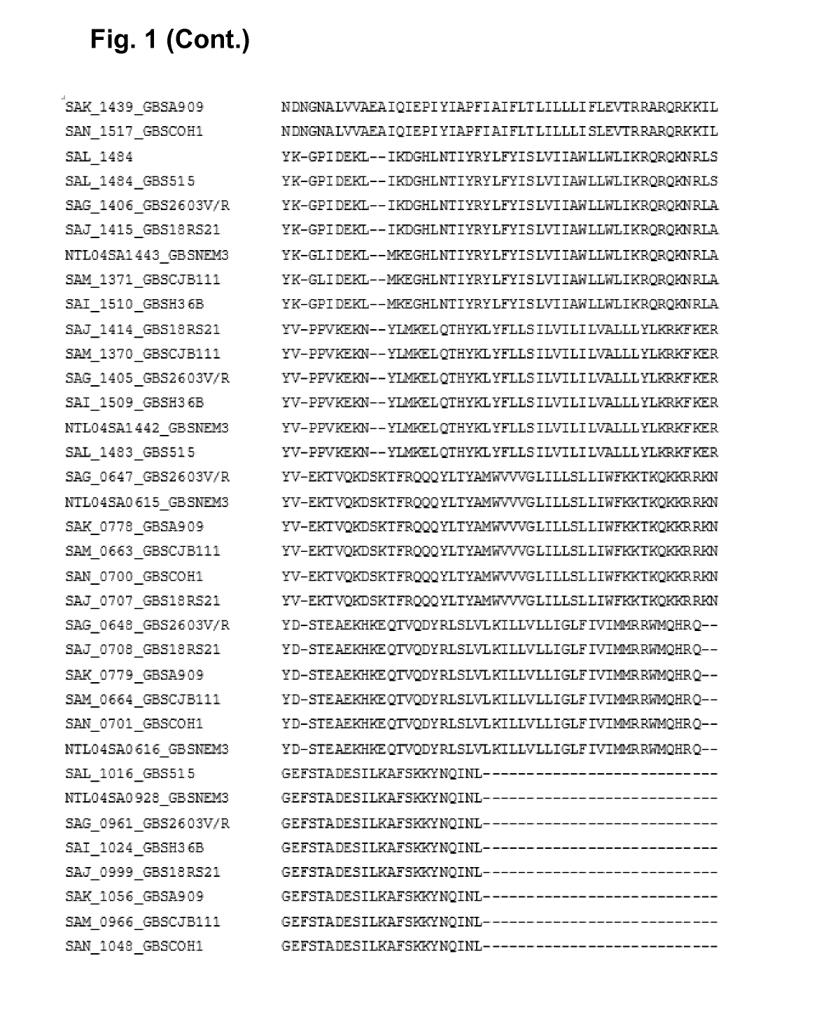

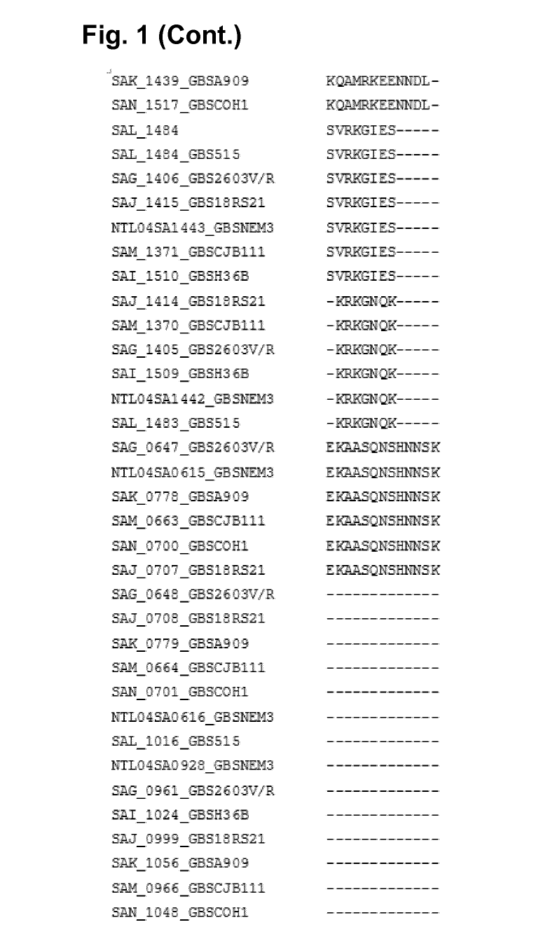

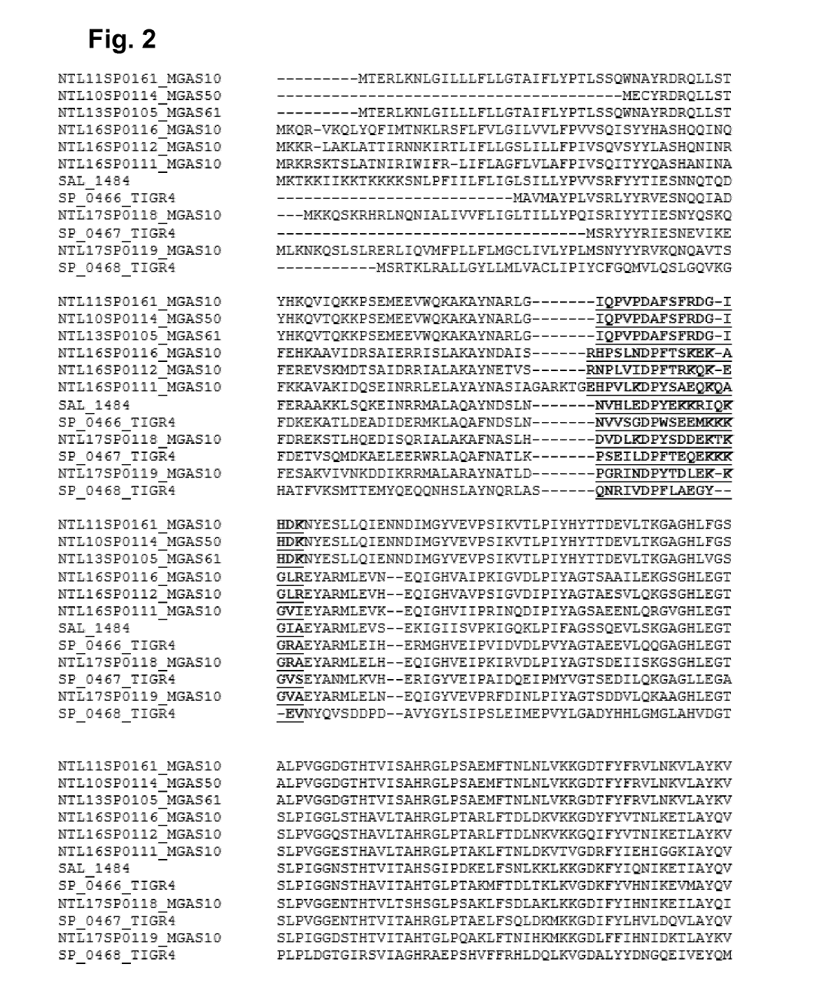

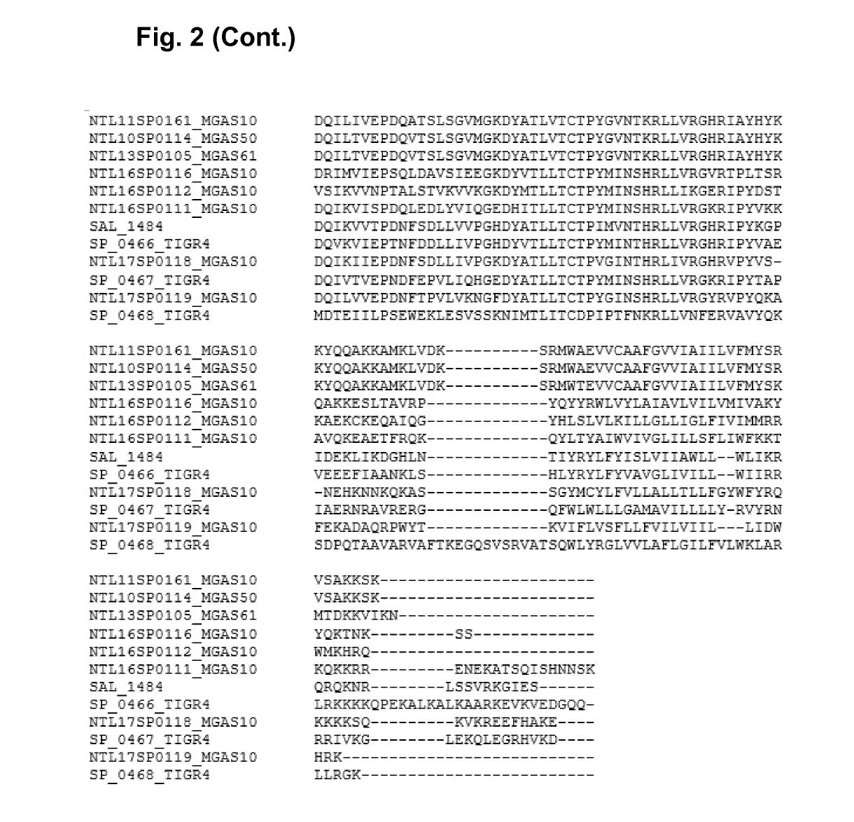

The location of the lid region in other Streptococcal sortase C enzymes can readily be determined by the skilled person by structural analysis or more simply, by alignment of the sequences of these enzyme with the sequences of the Streptococcal proteins having lid regions at known locations shown in Table 1. FIG. 1 provides an alignment of GBS sortase C enzymes highlighting the location of the lid regions. FIG. 2 provides a similar alignment for sortase C enzymes from GAS and pneumococcus. Any of the sortase C enzymes shown in these Figures having a lid region may be used in the methods of the invention.

The sortase C enzyme from Streptococcus used in the methods of the invention comprises a mutation in its lid region. The mutation may be a substitution, deletion or insertion in the amino acid sequence of the lid region of the mutant sortase C-enzyme relative to the amino acid sequence of the wild-type sortase C enzyme.

Deletion Mutants

Where the mutation is a deletion, the mutation may comprise deletion of part or all of the lid region of the wild-type sortase C enzyme. The lid region is typically around 15-18 amino acids long and the mutation may comprise deletion of 1, 2, 3, 4, 5, 6, 7, 8, 9, 10, 11, 12, 13, 14, 15, 16, 17, 18 or more amino acids from the lid region, or deletion of all of the amino acids in the lid region.

The mutation may comprise deletion of amino acids at positions predicted to interact with the catalytic triad in the active site of the sortase C enzyme. For example, the mutation may comprise the deletion of amino acids at positions 84, 85 and/or 86 of the amino acid sequence of the GBS sortase C1 enzyme of PI-2a (SEQ ID NO:3), or the deletion of amino acids at corresponding positions in the amino acid sequence of other sortase C enzymes. The mutation may thus comprise the deletion of: i) an amino acid at position 84; ii) an amino acid at position 85; iii) an amino acid at position 86; iv) two amino acids at positions 84 and 85; v) two amino acids at positions 84 and 86; vi) two amino acids at positions 85 and 86; or vii) three amino acids at positions 84, 85 and 86 of the amino acid sequence of the GBS sortase C1 enzyme of PI-2a (SEQ ID NO:3), or the deletion of amino acids at corresponding positions in the amino acid sequence of another sortase C enzyme. Amino acids at positions corresponding to positions 84, 85 and 86 of the amino acid sequence of the GBS sortase C1 enzyme of PI-2a (SEQ ID NO:3) can readily be determined by alignment.

Amino acids at positions corresponding to positions 84, 85 and 86 of the amino acid sequence of the GBS sortase C1 enzyme of PI-2a (SEQ ID NO:3) are found at:
positions 90, 91 and 92 of the GBS sortase C1 of PI-1 (SEQ ID NO:1),
positions 84, 85 and 86 of the GBS sortase C2 of PI-1 (SEQ ID NO:2),
positions 88, 89 and 90 of the GBS sortase C2 of PI-2a (SEQ ID NO:4),
positions 53, 54 and 55 of the GBS sortase C1 of PI-2b (SEQ ID NO:5),
positions 58, 59 and 60 of the pneumococcal sortase C1 (SEQ ID NO:6),
positions 50, 51 and 52 of the pneumococcal sortase C2 (SEQ ID NO:7),
positions 74, 75 and 76 of the pneumococcal sortase C3 (SEQ ID NO:8), or
positions 46, 47 and 48 of the GAS sortase C1 (SEQ ID NO:9), respectively.

Alternatively, the mutation may comprise the deletion of all of amino acids in the lid region. The deletion may comprise further changes at positions within the remaining sortase sequence. For example, the sortase may comprise substitutions, deletions or insertions at 1, 2, 3, 4, 5, 6, 7, 8, 9, 10, 11, 12, 13, 14, 15, 16, 17, 18, 19, 20 or more additional amino acid positions. By way of further example, the sortase may comprise substitutions, deletions or insertions at fewer than, 2, 3, 4, 5, 6, 7, 8, 9, 10, 11, 12, 13, 14, 15, 16, 17, 18, 19, 20 additional amino acid positions or any range therebetween.

In particular, the mutation may additionally comprise deletion of part or all of the signal peptide and/or transmembrane domain of the wild-type sortase C enzyme which is N-terminal of the lid region in the wild-type enzyme. The transmembrane domain comprises two alpha-helices. The mutation may comprise deletion of one or both of these two alpha-helices and, optionally, may also comprise deletion of the signal peptide N-terminal of the transmembrane domain. For example, the mutation may comprise deletion of part or all of the lid region and the deletion of 10, 20, 30, 40, 50, 60, 70, 80, 90 or more amino acids N-terminal of the lid region. By way of further example, the mutation may comprise deletion of part or all of the lid region and the deletion of less than 10, 20, 30, 40, 50, 60, 70, 80, 90 amino acids N-terminal of the lid region or any range therebetween. In some embodiments, the mutation comprises the deletion of all of amino acids in the lid region and all of amino acids N-terminal of the lid region. The sortase C enzyme in this embodiment of the invention thus consists of the C-terminal/positively charged domain of the wild-type sortase C enzyme.

The mutation may consist of the deletions described above in the absence of any further mutations. For example, the mutation may consist of deletion of part or all of the lid region, deletion of part or all of the lid region and the signal peptide and/or transmembrane domain, or deletion of part or all of the lid region and the entre N-terminal region in the absence of any further mutations. Examples of sequences of sortase C enzymes where the mutation consists of a) deletion of all of the lid region and the signal peptide/transmembrane domain, b) deletion of all of the lid region and the entire N-terminal regions, and c) deletion of the signal peptide/transmembrane domain and amino acids in the catalytic triad which are suitable for use in the methods of the invention are provided in Table 2 below.

TABLE 2
Deletion mutants of sortase C enzymes

| | | | Sequence of mutant sortase |
|---|---|---|---|
| | | | with signal peptide/trans- |
| | Sequence of mutant sortase | Sequence of mutant sortase | membrane domain and amino acids |
| | with signal peptide/trans- | with entire N-terminal | corresponding to residues 84-86 |
| Sortase | membrane domain and lid deleted | regions and lid deleted | of GBS sortase C1 of P1-2a deleted |
| GBS sortase C1 of PI-1 | SEQ ID NO: 10 | SEQ ID NO: 19 | SEQ ID NO: 28 |
| GBS sortase C2 of PI-1 | SEQ ID NO: 11 | SEQ ID NO: 20 | SEQ ID NO: 29 |
| GBS sortase C1 of PI-2a | SEQ ID NO: 12 | SEQ ID NO: 21 | SEQ ID NO: 30 |
| GBS sortase C2 of PI-2a | SEQ ID NO: 13 | SEQ ID NO: 22 | SEQ ID NO: 31 |
| GBS sortase C1 of PI-2b | SEQ ID NO: 14 | SEQ ID NO: 23 | SEQ ID NO: 32 |
| Pneumococcus sortase C1 | SEQ ID NO: 15 | SEQ ID NO: 24 | SEQ ID NO: 33 |
| Pneumococcus sortase C2 | SEQ ID NO: 16 | SEQ ID NO: 25 | SEQ ID NO: 34 |
| Pneumococcus sortase C3 | SEQ ID NO: 17 | SEQ ID NO: 26 | SEQ ID NO: 35 |
| GAS sortase C1 | SEQ ID NO: 18 | SEQ ID NO: 27 | SEQ ID NO: 36 |

Mutant sortase enzymes used in the methods of the invention may thus comprise or consist of an amino acid sequence selected from SEQ ID NO: 10, 11, 12, 13, 14, 15, 16, 17, 18, 19, 20, 21, 22, 23, 24, 25, 26, 27, 28, 29, 30, 31, 32, 33, 34, 35, or 36. Mutant sortase enzymes used in the methods of the invention may also comprise or consist of an amino acid sequence selected from SEQ ID NO: 10, 11, 12, 13, 14, 15, 16, 17, 18, 19, 20, 21, 22, 23, 24, 25, 26, 27, 28, 29, 30, 31, 32, 33, 34, 35, or 36 except for the substitution, deletion or insertion of 1, 2, 3, 4, 5, 6, 7, 8, 9 or 10 amino acids.

Substitution Mutants

The mutation may comprise one or more amino acid substitutions in the lid region compared to the wild-type sortase C enzyme sequence. The substitution(s) may be at positions in the lid region predicted to interact with amino acids in the catalytic site such that the substitutions abolish normal lid function. The mutation may comprise the substitution of amino acids at positions 84, 85 and/or 86 of the amino acid sequence of the GBS sortase C1 enzyme of PI-2a (SEQ ID NO:3), or the substitution deletion of amino acids at corresponding positions in the amino acid sequence of other sortase C enzymes. The mutation may thus comprise the substitution of: i) an amino acid at position 84; ii) an amino acid at position 85; iii) an amino acid at position 86; iv) two amino acids at positions 84 and 85; v) two amino acids at positions 84 and 86; vi) two amino acids at positions 85 and 86; or vii) three amino acids at positions 84, 85 and 86 of the amino acid sequence of the GBS sortase C1 enzyme of PI-2a (SEQ ID NO:3), or the substitution of amino acids at corresponding positions in the amino acid sequence of another sortase C enzyme. Amino acids at positions corresponding to positions 84, 85 and 86 of the amino acid sequence of the GBS sortase C1 enzyme of PI-2a (SEQ ID NO:3) can readily be determined by alignment.

Amino acids at positions corresponding to positions 84, 85 and 86 of the amino acid sequence of the GBS sortase C1 enzyme of PI-2a (SEQ ID NO:3) are found at:
positions 90, 91 and 92 of the GBS sortase C1 of PI-1 (SEQ ID NO:1),
positions 84, 85 and 86 of the GBS sortase C2 of PI-1 (SEQ ID NO:2),
positions 88, 89 and 90 of the GBS sortase C2 of PI-2a (SEQ ID NO:4),
positions 53, 54 and 55 of the GBS sortase C1 of PI-2b (SEQ ID NO:5),
positions 58, 59 and 60 of the pneumococcal sortase C1 (SEQ ID NO:6),
positions 50, 51 and 52 of the pneumococcal sortase C2 (SEQ ID NO:7),
positions 74, 75 and 76 of the pneumococcal sortase C3 (SEQ ID NO:8), or
positions 46, 47 and 48 of the GAS sortase C1 (SEQ ID NO:9), respectively.

The substitutions at positions corresponding to position 84 and/or position 85 and/or position 86 may comprise replacement of the wild-type residue at these positions with an alanine residue.

Where the sortase is GBS sortase C1 of PI-1 (SEQ ID NO:1), the mutation may comprise replacement of the aspartate residue at position 90 with an alanine residue (D90A) and/or replacement of the proline residue at position 91 with an alanine residue (P91A), and/or replacement of the tyrosine residue at position 92 with an alanine residue (Y92A).

Where the sortase is GBS sortase C2 of PI-1 (SEQ ID NO:2), the mutation may comprise replacement of the aspartate residue at position 84 with an alanine residue (D84A) and/or replacement of the proline residue at position 85 with an alanine residue (P85A), and/or replacement of the phenylalanine residue at position 86 with an alanine residue (F86A).

Where the sortase is GBS sortase C1 of PI-2a (SEQ ID NO:3), the mutation may comprise replacement of the aspartate residue at position 84 with an alanine residue (D84A) and/or replacement of the proline residue at position 85 with an alanine residue (P85A), and/or replacement of the tyrosine residue at position 86 with an alanine residue (Y86A).

Where the sortase is GBS sortase C2 of PI-2a (SEQ ID NO:4), the mutation may comprise replacement of the aspartate residue at position 88 with an alanine residue (D88A) and/or replacement of the proline residue at position 89 with an alanine residue (P89A), and/or replacement of the tyrosine residue at position 90 with an alanine residue (Y90A).

Where the sortase is GBS sortase C1 of PI-2b (SEQ ID NO:5), the mutation may comprise replacement of the methionine residue at position 53 with an alanine residue (M53A) and/or replacement of the lysine residue at position 54 with an alanine residue (K54A), and/or replacement of the tryptophan residue at position 55 with an alanine residue (W55A).

Where the sortase is pneumococcal sortase C1 (SEQ ID NO:6), the mutation may comprise replacement of the aspartate residue at position 58 with an alanine residue (D58A) and/or replacement of the proline residue at position 59 with an alanine residue (P59A), and/or replacement of the tryptophan residue at position 60 with an alanine residue (W55A).

Where the sortase is pneumococcal sortase C2 (SEQ ID NO:7), the mutation may comprise replacement of the aspartate residue at position 50 with an alanine residue (D50A) and/or replacement of the proline residue at position 51 with an alanine residue (P51A), and/or replacement of the phenylalanine residue at position 52 with an alanine residue (F52A).

Where the sortase is pneumococcal sortase C2 (SEQ ID NO:8), the mutation may comprise replacement of the aspartate residue at position 74 with an alanine residue (D74A) and/or replacement of the proline residue at position 75 with an alanine residue (P75A), and/or replacement of the phenylalanine residue at position 76 with an alanine residue (F76A).

Where the sortase is GAS sortase C1 (SEQ ID NO:9), the mutation may comprise replacement of the aspartate residue at position 46 with an alanine residue (D46A) and/or and/or replacement of the phenylalanine residue at position 48 with an alanine residue (F48A). The GAS sortase C1 enzyme already comprises an alanine residue at position 47.

The mutation may comprise further amino acid changes at positions other than at positions corresponding to positions 84 and/or 85 and/or 86 of the amino acid sequence of the GBS sortase C1 enzyme of PI-2a (SEQ ID NO:3). For example, the mutation may comprise substitutions at 1, 2, 3, 4, 5, 6, 7, 8, 9, 10, 11, 12, 13, 14, 15, 16, 17, 18, 19, 20 or more additional amino acid positions. Alternatively or in addition to these further substitutions, the mutation may comprise deletions and/or insertions. In particular, the mutation may comprise substitutions at positions corresponding to positions 84 and/or 85 and/or 86 of the amino acid sequence of the GBS sortase C1 enzyme of PI-2a (SEQ ID NO:3) and deletion of a) the signal peptide and/or transmembrane domain, or b) deletion of the entire N-terminal region of the wild-type sortase enzyme.

The sortase may consist of substitutions at positions 84 and/or 85 and/or 86 in the absence of any further mutations. Examples of sequences of sortase C enzymes consisting of substitutions at positions that are equivalent to positions 84 and/or 86 of the lid region of the amino acid sequence of the GBS sortase C1 enzyme of PI-2a (SEQ ID NO:3) and also consisting of deletion of the signal peptide/transmembrane region which are suitable for use in the methods of the invention are provided in Table 3 below.

TABLE 3
Substitution mutants of sortase C enzymes

| | Sequence of sortase with | Sequence of sortase with | Sequence of sortase with | Sequence of sortase with |
|---|---|---|---|---|
| | mutation corresponding to | mutation corresponding to | mutations corresponding to | mutations corresponding to |
| | position 84 of | position 86 of | positions 84 and 86 of | positions 84, 85 and 86 of |
| | GBS sortase C1 of P1-2a and | GBS sortase C1 of P1-2a and | GBS sortase C1 of P1-2a and | GBS sortase C1 of P1-2a and |
| | deletion of signal peptide/ | deletion of signal peptide/ | deletion of signal peptide/ | deletion of signal peptide/ |
| Sortase | transmembrane domain | transmembrane domain | transmembrane domain | transmembrane domain |
| GBS sortase | SEQ ID NO: 37 | SEQ ID NO: 46 | SEQ ID NO: 55 | SEQ ID NO: 64 |
| C1 of PI-1 | | | | |
| GBS sortase | SEQ ID NO: 38 | SEQ ID NO: 47 | SEQ ID NO: 56 | SEQ ID NO: 65 |
| C2 of PI-1 | | | | |
| GBS sortase | SEQ ID NO: 39 | SEQ ID NO: 48 | SEQ ID NO: 57 | SEQ ID NO: 66 |
| C1 of PI-2a | | | | |
| GBS sortase | SEQ ID NO: 40 | SEQ ID NO: 49 | SEQ ID NO: 58 | SEQ ID NO: 67 |
| C2 of PI-2a | | | | |
| GBS sortase | SEQ ID NO: 41 | SEQ ID NO: 50 | SEQ ID NO: 59 | SEQ ID NO: 68 |
| C1 of PI-2b | | | | |
| Pneumococcus | SEQ ID NO: 42 | SEQ ID NO: 51 | SEQ ID NO: 60 | SEQ ID NO: 69 |
| sortase C1 | | | | |
| Pneumococcus | SEQ ID NO: 43 | SEQ ID NO: 52 | SEQ ID NO: 61 | SEQ ID NO: 70 |
| sortase C2 | | | | |
| Pneumococcus | SEQ ID NO: 44 | SEQ ID NO: 53 | SEQ ID NO: 62 | SEQ ID NO: 71 |
| sortase C3 | | | | |
| GAS sortase C1 | SEQ ID NO: 45 | SEQ ID NO: 54 | SEQ ID NO: 63 | n/a |

Mutant sortase enzymes used in the methods of the invention may thus comprise or consist of an amino acid sequence selected from SEQ ID NO: 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70 or 71. Mutant sortase enzymes used in the methods of the invention may also comprise or consist of an amino acid sequence selected from SEQ ID NO:37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70 or 71 except for the substitution, deletion or insertion of 1, 2, 3, 4, 5, 6, 7, 8, 9 or 10 amino acids.

The mutant sortase C enzymes suitable for use in the methods of the invention described above are also embodiments of the invention in their own right. Particularly sortase mutants are the SrtC1_{Y92A} and SrtC2_{F86A} because the stability of these enzymes is higher, they are better expressed and more soluble in comparison with, for example SrtC1-ΔNT and SrtC2-ΔNT deletion mutants. This is surprising since the Vmax of the cleavage reaction for the Y92A and F86A mutants was lower than that of the SrtC1-ΔNT and SrtC2-ΔNT mutants which are also more difficult to purify.

Sortase Action

Sortases cleave the LPXTG motif of, for example, pilin proteins and covalently join the C terminus of one moiety, such as a pilin subunit, to a Lys side-chain NH2 group on the next moiety or subunit. Two recognition events are involved in this sortase action. Firstly, the sortase recognition motif (LPXTG or a variant) of the substrate protein must be recognised and bound. Secondly, the acceptor substrate, to which the substrate protein will be transferred, must be recognised and bound, and a specific amino group brought into position to attack the thioacyl intermediate.

Bacterial Polypeptides Polymerised by the Mutant Sortase C Enzymes

The mutant sortase C enzymes described above may be used to polymerise one or more polypeptides. The mutant sortase C enzymes are brought into contact with the one or more polypeptides in vitro and following a period of incubation, polymerised polypeptides are detected, for example by identifying a pattern of high molecular weight bands on SDS gels. Incubation may be carried out at 37° C. Incubation may be carried out for 1, 2, 3, 4, 5, 6, 7 days or more. The polypeptides and the mutant sortase C enzymes may be incubated in the presence of a reducing agent, for example DTT 1 mM, to keep the catalytic cysteine of the mutant sortase C enzyme active. Incubation may be carried out at around pH 7-8.

In contrast to the mutant sortase C enzymes of the invention, the wild-type sortase C enzymes fail to polymerise polypeptides in vitro. For the avoidance of doubt, use of the term “in vitro” refers to the use of isolated and/or purified components of a cell, such as an enzyme, to effect pilus polymerisation without requiring the presence of the cell itself.

The polypeptides polymerised by the mutant sortase C enzymes of the invention typically comprise the LPxTG motif. They may further comprise a pilin motif (consensus WxxxVxVyPK) and/or an E-Box motif (consensus YxLxETxAPxGY) shown to be important for pilus assembly [6]. In particular, the polypeptides may comprise a conserved lysine (K) residues, for example, found in the pilin motif. In other embodiments the polypeptides do not comprise a conserved lysine (K) residue in the pilin motif, i.e. wherein the presence of the conserved lysine residue is excluded. In some embodiments, the polypeptides polymerised by the mutant sortase C enzymes of the invention may comprise an N-terminal glycine residue. Other sequence motifs will be apparent to one skilled in the art and may include, by way of non-limiting example: LPETGG, LPXT, LPXTG, LPKTG, LPATG, LPNTG, IPQTG, IQTGGIGT.

Examples of polypeptides that may be polymerised by the mutant sortase C enzymes of the invention include polypeptides from Gram-positive bacteria, such as the backbone proteins and ancillary proteins that are found in the pili of Gram-positive bacteria. In particular, the mutant sortase C enzyme may be brought into contact with a backbone protein found in a pilus from GBS, GAS or Streptococcus pneumoniae. For example, the mutant sortase C enzyme may be brought into contact with the backbone protein from GBS PI-1 (GBS80/SAG0645), the backbone protein from GBS PI-2a (GBS59/SAG1407), the backbone protein from GBS PI-2b (Spb1/SAN1518), the backbone protein from Streptococcus pneumoniae (RrgB), or the backbone protein from GAS (fee6, spy128, orf80, eftLSLA).

Alternatively or in addition, the mutant sortase C enzyme may be brought into contact with an ancillary protein found in a pilus from GBS, GAS or Streptococcus pneumoniae. For example, the mutant sortase C enzyme may be brought into contact with the ancillary protein 1 (AP-1) from GBS PI-1 (GBS104), the AP-1 from GBS PI-2a (GBS67/SAG1408), the AP-1 from GBS PI-2b (SAN1519), the AP-1 from Streptococcus pneumoniae (RrgA) or the AP-1 from GAS (cpa), the ancillary protein 2 (AP-2) from GBS PI-1 (GBS52), the AP-2 from GBS PI-2a (GBS150/SAG1404), the AP-2 from GBS PI-2b (SAN1516), the AP-2 from Streptococcus pneumoniae (RrgC) or the AP-2 from GAS spy130, orf82, orf2).

The mutant sortase C enzymes of the invention may be used to polymerise homologues, fragments or variants of the wild-type backbone protein and ancillary protein sequences, provided that these homologues, fragments and variants retain the sequences described above necessary for polymerisation by mutant sortase C enzymes. For example, variants of these polypeptides that may be used in the methods of the invention include backbone proteins and/or ancillary protein sequences from which the transmembrane domain has been deleted compared to the wild-type sequence. In addition or instead of the deletion of the transmembrane domain, variants may comprise the additional of a glycine residue at the N-terminal to promote polymerisation.

By way of non-limiting example, the sequences some of these polypeptides which may be polymerised by the mutant sortase enzymes of the invention are provided below for reference. The sequences of additional polypeptides which may be polymerised by the mutant sortases of the invention can be readily determined by the skilled person. Further details of these polypeptides are provided in reference [7].

BP from PI-1 (GBS80)

The amino acid sequence of full length GBS80 as found in the 2603 strain is given as SEQ ID NO: 72 herein. Wild-type GBS80 contains a N-terminal leader or signal sequence region at amino acids 1-37 of SEQ ID NO:72. One or more amino acids from the leader or signal sequence region of GBS80 can be removed, e.g. SEQ ID NO:73.

BP from PI-2b (GBS1523/SAN1518)

The original ‘GBS1523’ (SAN1518; Spb1) sequence was annotated as a cell wall surface anchor family protein (see GI: 77408651). For reference purposes, the amino acid sequence of full length GBS 1523 as found in the COH1 strain is given as SEQ ID NO: 110 herein. Preferred GBS1523 polypeptides for use with the invention comprise an amino acid sequence: (a) having 60% or more identity (e.g. 60%, 65%, 70%, 75%, 80%, 85%, 90%, 91%, 92%, 93%, 94%, 95%, 96%, 97%, 98%, 99%, 99.5% or more) to SEQ ID NO:110; and/or (b) comprising a fragment of at least ‘n’ consecutive amino acids of SEQ ID NO: 110, wherein ‘n’ is 7 or more (e.g. 8, 10, 12, 14, 16, 18, 20, 25, 30, 35, 40, 50, 60, 70, 80, 90, 100, 150, 200, 250 or more).

The wild-type sequence contains an amino acid motif indicative of a cell wall anchor (LPSTG) at amino acids 468-472 of SEQ ID NO:110. An E box containing a conserved glutamic residue has also been identified at amino acids 419-429 of SEQ ID NO:110, with a conserved glutamic acid at residue 423. The E box motif may be important for the formation of oligomeric pilus-like structures, and so useful fragments of GBS1523 may include the conserved glutamic acid residue. A mutant of GBS1523 has been identified in which the glutamine (Q) at position 41 of SEQ ID NO:110 is substituted for a lysine (K), as a result of a mutation of a codon in the encoding nucleotide sequence from CAA to AAA. This substitution may be present in the GBS1523 sequences and GBS1523 fragments (e.g. SEQ ID NO:112). A further variant of GBS1523 COH1 without signal sequence region is provided as SEQ ID NO:111.

BP from GBS PI-2a (GBS59)

The amino acid sequence of full length GBS59 as found in the 2603 strain is given as SEQ ID NO: 74 herein. Variants of GBS59 exist in strains H36B, 515, CJB111, DK21 and CJB110. The amino acid sequence of full length GBS59 as found in the H36B, 515, CJB111, CJB110 and DK21 strains are given as SEQ ID NOs: 75, 76, 77, 78, and 79.

BP from GBS PI-2b (Spb1)

The amino acid sequence of full length Sbp1 as found in the COH1 strain is given as SEQ ID NO:80 herein. Wild-type Spb1 contains a N-terminal leader or signal sequence region. One or more amino acids from the leader or signal sequence region of Spb1 can be removed, e.g. SEQ ID NO:81.

BP from Streptococcus pneumoniae (RrgB)

The RrgB pilus subunit has at least three clades. Reference amino acid sequences for the three clades are SEQ ID NOs: 82, 83 and 84 herein.

AP-1 from GBS PI-1 (GBS104/SAG0649)

The amino acid sequence of full length GBS104 as found in the 2603 strain is given as SEQ ID NO:85 herein.

AP-1 from GBS PI-2a (GBS67)

The amino acid sequence of full length GBS67 as found in the 2603 strain is given as SEQ ID NO: 86 herein. A variant of GBS67 (SAI1512) exists in strain H36B. The amino acid sequence of full length GBS67 as found in the H36B strain is given as SEQ ID NO: 87. Variants of GBS67 also exists in strains CJB111, 515, NEM316, DK21 and CJB110. The amino acid sequences of full length GBS67 as found in the CJB111, 515, NEM316, DK21 and CJB110 strains are given as SEQ ID NOS: 88, 89, 90, 91, and 92 herein.

AP-1 from GBS PI-2b (GBS1524/SAN1519)

The amino acid sequence of full length GBS1524 (SAN1519) as found in the COH1 strain is given as SEQ ID NO:93 herein.

AP-1 from Streptococcus pneumoniae (RrgA)

The amino acid sequence of full length RrgA is given as SEQ ID NO:94 herein.

AP-2 from GBS PI-1 (GBS052/SAG0646)

The amino acid sequence of full length GBS052/SAG0646 as found in the 2603 strain is given as SEQ ID NO:95 herein.

AP-2 from GBS PI-2a (GBS150/SAG1404)

The amino acid sequence of full length GBS150/SAG1404 as found in the 2603 strain is given as SEQ ID NO:96 herein.

AP-2 from Streptococcus pneumonia (RrgC)

The amino acid sequence of full length RrgC is given as SEQ ID NO:97 herein.

Polypeptides for use with the invention may thus comprise or consist of an amino acid sequence: (a) having 50% or more identity (e.g. 60%, 65%, 70%, 75%, 80%, 85%, 90%, 91%, 92%, 93%, 94%, 95%, 96%, 97%, 98%, 99%, 99.5% or more) to a polypeptide having the amino acid sequence of any one of SEQ ID NOs: 72, 73, 74, 75, 76, 77, 78, 79, 80, 81, 82, 83, 84, 85, 86, 87, 88, 89, 90, 91, 92, 93, 94, 95, 96, or 97 or to any other backbone or ancillary protein sequences described above; or (b) that is a fragment of at least ‘n’ consecutive amino acids of one of these sequences wherein ‘n’ is 20 or more (e.g. 25, 30, 35, 40, 50, 60, 70, 80, 90, 100, 150 or more; e.g. 20 or more; or e.g. 50 or more; or e.g. 80 or more). Alternatively, ‘n’ is less than 20 or less than 25, 30, 35, 40, 50, 60, 70, 80, 90, 100 or less than 150.

The methods of the invention may involve polymerisation of at least 1, 2, 3, 4, 5, 6, 7, 8, 9, 10, 11, 12, 13, 14, 15, 16 or 17 polypeptides having 50% identity to a polypeptide having the amino acid sequence of any one of SEQ ID NOs: 72, 73, 74, 75, 76, 77, 78, 79, 80, 81, 82, 83, 84, 85, 86, 87, 88, 89, 90, 91, 92, 93, 94, 95, 96, or 97, or of or of fragments of at least ‘n’ consecutive amino acids of one of these sequences wherein ‘n’ is 20 or more (e.g. 25, 30, 35, 40, 50, 60, 70, 80, 90, 100, 150 or more; e.g. 20 or more; or e.g. 50 or more; or e.g. 80 or more).

The methods of the invention may involve polymerisation of 1, 2, 3, 4, 5 or 6 polypeptides having 50% identity e.g. 60%, 65%, 70%, 75%, 80%, 85%, 90%, 91%, 92%, 93%, 94%, 95%, 96%, 97%, 98%, 99%, 99.5% or more) to a polypeptide having the amino acid sequence of any one of SEQ ID NOs: 74, 75, 76, 77, 78 and 79, or of fragments of at least ‘n’ consecutive amino acids of one of these sequences wherein ‘n’ is 20 or more (e.g. 25, 30, 35, 40, 50, 60, 70, 80, 90, 100, 150 or more; e.g. 20 or more; or e.g. 50 or more; or e.g. 80 or more).

The methods of the invention may involve polymerisation of 1, 2, or 3 polypeptides having 50% identity e.g. 60%, 65%, 70%, 75%, 80%, 85%, 90%, 91%, 92%, 93%, 94%, 95%, 96%, 97%, 98%, 99%, 99.5% or more) to a polypeptide having the amino acid sequence of any one of SEQ ID NOs: 82, 83 and 84, or of fragments of at least ‘n’ consecutive amino acids of one of these sequences wherein ‘n’ is 20 or more (e.g. 25, 30, 35, 40, 50, 60, 70, 80, 90, 100, 150 or more; e.g. 20 or more; or e.g. 50 or more; or e.g. 80 or more).

Amino acid fragments of these backbone and ancillary proteins may comprise an amino acid sequence of e.g up to 30, up to 40, up to 50, up to 60, up to 70, up to 80, up to 90, up to 100, up to 125, up to 150, up to 175, up to 200, up to 250, up to 300, up to 350, up to 400, up to 450, up to 500, up to 550, up to 600, up to 650, up to 700, up to 750, up to 800, up to 850, up to 900, up to 950, up to 1000, up to 1100, up to 1200, up to 1300, up to 1400, up to 1500, consecutive amino acid residues of the sequences provided above. Other fragments omit one or more polypeptide domains, for example the transmembrane domain.

The mutant sortase C enzymes of the invention polymerise these polypeptides in a manner that is analogous to the polymerisation of backbone proteins and accessory proteins by wild-type Streptococcal sortase C enzymes in vivo to form a pilus. The polymerised polypeptides produced according to these methods are thus structurally similar to a pilus produced by a Streptococcal bacterium in vivo.

Pili in Gram-positive bacteria are constructed from either two or three types of pilin subunits. In two-component pili the shaft of the pilus is formed by multiple copies of a major pilin subunit, while the tip of the pilus contains a single copy of a minor ‘tip’ pilin subunit that typically functions as an adhesin. Three-component pili are similar, but they also contain a minor ‘basal’ pilin subunit that is covalently attached to the cell wall. Several transmission electron microscopy (EM) and immuno-gold labelling studies have led to the conclusion that the minor ‘basal’ pilin subunits are also interspersed throughout the shaft of the pilus, presumably because the sortase enzymes are promiscuous in the substrates they recognize.

The mutant sortase C enzymes may be brought into contact with 1 polypeptide, leading to the formation of a monomeric pilus. For example, the mutant sortase enzyme may be brought into contact with GBS80, GBS59 or RrgB, leading to the formation of a monomeric pilus comprising subunits of GBS80, GBS59 or RrgB respectively. Where the polypeptide is from a Gram positive bacterium, the mutant sortase enzyme that is used to polymerise that polypeptide need not be from the same Gram positive bacterium. Thus, a mutant sortase C enzyme derived from GBS can be used to polymerise proteins not just from GBS but also from Streptococcus pneumoniae and/or GAS. Variants of some pilus proteins, such as GBS59 are not generally cross-protective. Therefore, the ability to polymerise combinations of at least 2, 3, 4, 5, 6 or more of these variants within an individual pilus is advantageous, for example avoiding the need for more complex compositions or use of protein fusions to achieve cross-protection. Particularly, pili polymerised in vitro may include a combination of GBS59 variants from GBS strains 515, CJB111, H36B, 2603, DK21 and 090, more particularly a combination of GBS59 variants from GBS strains 515, CJB111, H36B and 2603. Such pili comprising two or more variants of GBS59 are not found in nature because strains of wild type bacteria express only one variant of back-bone protein (BP-2a/GBS59).

Alternatively, the mutant sortase C enzymes may be brought into contact with 2, 3, 4, 5 or more different polypeptides which may be from 1, 2, 3, 4, 5 or more Gram positive bacteria, leading to the formation of a chimeric pilus. The mutant sortase C enzymes may be brought into contact with the backbone and accessory proteins from a single Gram positive bacterium which are found in combination in a natural Streptococcal pilus from that bacterium, resulting in a chimeric pilus that is equivalent in structure to a naturally-occurring pilus. Such chimeric pili are a useful tool to enable the study of pilus properties without the laborious purification process currently used to isolate pili from Gram positive bacteria.

In addition, as discussed above, the three-dimensional structures of the monomeric and chimeric pili produced by the methods of the invention make them particularly convenient and effective for immunisation purposes compared to the administration of individual recombinant proteins. Indeed, protection assays have shown that these pili are more effective at inducing protection against the Streptococcus bacteria from which they are derived than monomeric recombinant proteins. It is postulated that this may be because the pili contain epitopes present in pili in vivo that are not replicated in monomeric recombinant proteins, particularly such epitopes are structural epitopes.

The invention includes pili obtained or obtainable using the methods of the invention. In some aspects, the combinations of polypeptides found in these pili differ from the combination of polypeptides found in naturally-occurring pili in Streptococcal bacteria. Examples of pili that may be produced according to the methods of the invention include pili comprising or consisting of the backbone proteins and/or the ancillary proteins from Streptococcus described above. In some embodiments, these pili do not contain the combinations of polypeptides found in naturally-occurring pili found in GBS, GAS or Streptococcal pneumoniae. Particularly, pili polymerised in vitro differ from naturally-occurring pili in terms of their composition, for example, because the acyl enzyme intermediate is not attached to a wild type sortase but is attached to a mutant sortase of the invention. In other cases, pili polymerised in vitro do not comprise cell wall/membrane components such as lipid II or precursors of peptidoglycan such as MurNAc—N-acetyl-muramic acid. In yet other cases, pili polymerised in vitro comprise combinations of pilus proteins not found in nature. Thus, pili polymerised in vitro can be differentiated from those occurring naturally. Thus, the term “artificial” refers to a synthetic, or non-cell derived composition, particularly a structure which is synthesized in vitro and which is not identical to structures found in native bacteria such as Streptococcus.

Immunogenic Compositions Comprising Pili

The invention provides immunogenic compositions comprising the pili described above, which may be obtained or obtainable by the methods of the invention. The term “immunogenic” is used to mean that the pilus is capable of eliciting an immune response, such as a cell-mediated and/or an antibody response, against the polypeptide or polypeptides making up the pilus when used to immunise a subject (preferably a mammal, more preferably a human or a mouse). Particularly, the immune response is a protective immune response which provides protective immunity.

Immunogenic compositions of the invention may be useful as vaccines. Vaccines according to the invention may either be prophylactic (i.e. to prevent infection) or therapeutic (i.e. to treat infection), but will typically be prophylactic. Prophylactic vaccines do not guarantee complete protection from disease because even if the patient develops antibodies, there may be a lag or delay before the immune system is able to fight off the infection. Therefore, and for the avoidance of doubt, the term prophylactic vaccine may also refer to vaccines that ameliorate the effects of a future infection, for example by reducing the severity or duration of such an infection.

The terms “protection against infection” and/or “provide protective immunity” means that the immune system of a subject has been primed (e.g by vaccination) to trigger an immune response and repel infection. Particularly, the immune response triggered is capable of repelling infection against a number of different strains of bacteria. A vaccinated subject may thus get infected, but is better able to repel the infection than a control subject.

Compositions may thus be pharmaceutically acceptable. They will usually include components in addition to the antigens e.g. they typically include one or more pharmaceutical carrier(s) and/or excipient(s). A thorough discussion of such components is available in reference [8].

Compositions will generally be administered to a mammal in aqueous form. Prior to administration, however, the composition may have been in a non-aqueous form. For instance, although some vaccines are manufactured in aqueous form, then filled and distributed and administered also in aqueous form, other vaccines are lyophilised during manufacture and are reconstituted into an aqueous form at the time of use. Thus a composition of the invention may be dried, such as a lyophilised formulation.

The composition may include preservatives such as thiomersal or 2-phenoxyethanol. It is preferred, however, that the vaccine should be substantially free from (i.e. less than 5 μg/ml) mercurial material e.g. thiomersal-free. Vaccines containing no mercury are more preferred. Preservative-free vaccines are particularly preferred.

To improve thermal stability, a composition may include a temperature protective agent. Further details of such agents are provided below.

To control tonicity, it is preferred to include a physiological salt, such as a sodium salt. Sodium chloride (NaCl) is preferred, which may be present at between 1 and 20 mg/ml e.g. about 10±2 mg/ml NaCl. Other salts that may be present include potassium chloride, potassium dihydrogen phosphate, disodium phosphate dehydrate, magnesium chloride, calcium chloride, etc.

Compositions will generally have an osmolality of between 200 mOsm/kg and 400 mOsm/kg, preferably between 240-360 mOsm/kg, and will more preferably fall within the range of 290-310 mOsm/kg.

Compositions may include one or more buffers. Typical buffers include: a phosphate buffer; a Tris buffer; a borate buffer; a succinate buffer; a histidine buffer (particularly with an aluminum hydroxide adjuvant); or a citrate buffer. Buffers will typically be included in the 5-20 mM range.

The pH of a composition will generally be between 5.0 and 8.1, and more typically between 6.0 and 8.0 e.g. 6.5 and 7.5, or between 7.0 and 7.8.

The composition is preferably sterile. The composition is preferably non-pyrogenic e.g. containing <1 EU (endotoxin unit, a standard measure) per dose, and preferably <0.1 EU per dose. The composition is preferably gluten free.

The composition may include material for a single immunisation, or may include material for multiple immunisations (i.e. a ‘multidose’ kit). The inclusion of a preservative is preferred in multidose arrangements. As an alternative (or in addition) to including a preservative in multidose compositions, the compositions may be contained in a container having an aseptic adaptor for removal of material.

Human vaccines are typically administered in a dosage volume of about 0.5 ml, although a half dose (i.e. about 0.25 ml) may be administered to children.

Immunogenic compositions of the invention may also comprise one or more immunoregulatory agents. Preferably, one or more of the immunoregulatory agents include one or more adjuvants. The adjuvants may include a TH1 adjuvant and/or a TH2 adjuvant, further discussed below.

Adjuvants which may be used in compositions of the invention include, but are not limited to:
mineral salts, such as aluminium salts and calcium salts, including hydroxides (e.g. oxyhydroxides), phosphates (e.g. hydroxyphosphates, orthophosphates) and sulphates, etc. [e.g. see chapters 8 & 9 of ref 9];
oil-in-water emulsions, such as squalene-water emulsions, including MF59 (5% Squalene, 0.5% Tween 80, and 0.5% Span 85, formulated into submicron particles using a microfluidizer) [Chapter 10 of ref.9, see also ref. 10-13, chapter 10 of ref. 14 and chapter 12 of ref. 15], complete Freund's adjuvant (CFA) and incomplete Freund's adjuvant (IFA);
saponin formulations [chapter 22 of ref. 9], such as QS21 [16] and ISCOMs [chapter 23 of ref. 9];
virosomes and virus-like particles (VLPs) [17-23];
bacterial or microbial derivatives, such as non-toxic derivatives of enterobacterial lipopolysaccharide (LPS), Lipid A derivatives [24, 25], immunostimulatory oligonucleotides [26-31], such as IC-31™ [32] (deoxynucleotide comprising 26-mer sequence 5′-(IC)₁₃-3′ (SEQ ID NO: 46) and polycationic polymer peptide comprising 11-mer amino acid sequence KLKLLLLLKLK (SEQ ID NO: 47)) and ADP-ribosylating toxins and detoxified derivatives thereof [33-42];
human immunomodulators, including cytokines, such as interleukins (e.g. IL-1, IL-2, IL-4, IL-5, IL-6, IL-7, IL-12 [43, 44], interferons (e.g. interferon-γ), macrophage colony stimulating factor, and tumor necrosis factor;
bioadhesives and mucoadhesives, such as chitosan and derivatives thereof, esterified hyaluronic acid microspheres [45] or mucoadhesives, such as cross-linked derivatives of poly(acrylic acid), polyvinyl alcohol, polyvinyl pyrollidone, polysaccharides and carboxymethylcellulos [46];
microparticles (i.e. a particle of ˜100 nm to ˜150 μm in diameter, more preferably ˜200 nm to ˜30 μm in diameter, and most preferably ˜500 nm to ˜10 μm in diameter) formed from materials that are biodegradable and non-toxic (e.g. a poly(a-hydroxy acid), a polyhydroxybutyric acid, a polyorthoester, a polyanhydride, a polycaprolactone, etc.);
liposomes [Chapters 13 & 14 of ref. 9, ref. 47-49]; polyoxyethylene ethers and polyoxyethylene esters [50];
PCPP formulations [51 and 52];
muramyl peptides, including N-acetyl-muramyl-L-threonyl-D-isoglutamine (thr-MDP), N-acetyl-normuramyl-1-alanyl-d-isoglutamine (nor-MDP), and N-acetylmuramyl-1-alanyl-d-isoglutaminyl-1-alanine-2-(1′-2′-dipalmitoyl-sn-glycero-3-hydroxyphosphoryloxy)-ethylamine MTP-PE); and
imidazoquinolone compounds, including Imiquamod and its homologues (e.g. “Resiquimod 3M”) [53 and 54].

Immunogenic compositions and vaccines of the invention may also comprise combinations of aspects of one or more of the adjuvants identified above. For example, the following adjuvant compositions may be used in the invention: (1) a saponin and an oil-in-water emulsion [55]; (2) a saponin (e.g. QS21)+a non-toxic LPS derivative (e.g. 3dMPL) [56]; (3) a saponin (e.g. QS21)+a non-toxic LPS derivative (e.g. 3dMPL)+a cholesterol; (4) a saponin (e.g. QS21)+3dMPL+IL-12 (optionally+a sterol) [57]; (5) combinations of 3dMPL with, for example, QS21 and/or oil-in-water emulsions [58]; (6) SAF, containing 10% squalane, 0.4% Tween 80™, 5% pluronic-block polymer L121, and thr-MDP, either microfluidized into a submicron emulsion or vortexed to generate a larger particle size emulsion. (7) Ribi™ adjuvant system (RAS), (Ribi Immunochem) containing 2% squalene, 0.2% Tween 80, and one or more bacterial cell wall components from the group consisting of monophosphorylipid A (MPL), trehalose dimycolate (TDM), and cell wall skeleton (CWS), preferably MPL+CWS (Detox™); and (8) one or more mineral salts (such as an aluminum salt)+a non-toxic derivative of LPS (such as 3dMPL). Other substances that act as immunostimulating agents are disclosed in chapter 7 of ref 9.

The use of an aluminium hydroxide and/or aluminium phosphate adjuvant is particularly preferred, and antigens are generally adsorbed to these salts. Calcium phosphate is another preferred adjuvant. Other preferred adjuvant combinations include combinations of Th1 and Th2 adjuvants such as CpG & alum or resiquimod & alum. A combination of aluminium phosphate and 3dMPL may be used (this has been reported as effective in pneumococcal immunisation [59]).

The compositions of the invention may elicit both a cell mediated immune response as well as a humoral immune response. This immune response will preferably induce long lasting (e.g. neutralising) antibodies and a cell mediated immunity that can quickly respond upon exposure to infection.

Two types of T cells, CD4 and CD8 cells, are generally thought necessary to initiate and/or enhance cell mediated immunity and humoral immunity. CD8 T cells can express a CD8 co-receptor and are commonly referred to as Cytotoxic T lymphocytes (CTLs). CD8 T cells are able to recognized or interact with antigens displayed on MHC Class I molecules.

CD4 T cells can express a CD4 co-receptor and are commonly referred to as T helper cells. CD4 T cells are able to recognize antigenic peptides bound to MHC class II molecules. Upon interaction with a MHC class II molecule, the CD4 cells can secrete factors such as cytokines. These secreted cytokines can activate B cells, cytotoxic T cells, macrophages, and other cells that participate in an immune response. Helper T cells or CD4+ cells can be further divided into two functionally distinct subsets: TH1 phenotype and TH2 phenotypes which differ in their cytokine and effector function.

Activated TH1 cells enhance cellular immunity (including an increase in antigen-specific CTL production) and are therefore of particular value in responding to intracellular infections. Activated TH1 cells may secrete one or more of IL-2, IFN-γ, and TNF-β. A TH1 immune response may result in local inflammatory reactions by activating macrophages, NK (natural killer) cells, and CD8 cytotoxic T cells (CTLs). A TH1 immune response may also act to expand the immune response by stimulating growth of B and T cells with IL-12. TH1 stimulated B cells may secrete IgG2a.

Activated TH2 cells enhance antibody production and are therefore of value in responding to extracellular infections. Activated TH2 cells may secrete one or more of IL-4, IL-5, IL-6, and IL-10. A TH2 immune response may result in the production of IgG1, IgE, IgA and memory B cells for future protection.

An enhanced immune response may include one or more of an enhanced TH1 immune response and a TH2 immune response.

A TH1 immune response may include one or more of an increase in CTLs, an increase in one or more of the cytokines associated with a TH1 immune response (such as IL-2, IFN-γ, and TNF-β), an increase in activated macrophages, an increase in NK activity, or an increase in the production of IgG2a. Preferably, the enhanced TH1 immune response will include an increase in IgG2a production.

A TH1 immune response may be elicited using a TH1 adjuvant. A TH1 adjuvant will generally elicit increased levels of IgG2a production relative to immunization of the antigen without adjuvant. TH1 adjuvants suitable for use in the invention may include for example saponin formulations, virosomes and virus like particles, non-toxic derivatives of enterobacterial lipopolysaccharide (LPS), immunostimulatory oligonucleotides. Immunostimulatory oligonucleotides, such as oligonucleotides containing a CpG motif, are preferred TH1 adjuvants for use in the invention.

A TH2 immune response may include one or more of an increase in one or more of the cytokines associated with a TH2 immune response (such as IL-4, IL-5, IL-6 and IL-10), or an increase in the production of IgG1, IgE, IgA and memory B cells. Preferably, the enhanced TH2 immune resonse will include an increase in IgG1 production.

A TH2 immune response may be elicited using a TH2 adjuvant. A TH2 adjuvant will generally elicit increased levels of IgG1 production relative to immunization of the antigen without adjuvant. TH2 adjuvants suitable for use in the invention include, for example, mineral containing compositions, oil-emulsions, and ADP-ribosylating toxins and detoxified derivatives thereof. Mineral containing compositions, such as aluminium salts are preferred TH2 adjuvants for use in the invention.

Preferably, the invention includes a composition comprising a combination of a TH1 adjuvant and a TH2 adjuvant. Preferably, such a composition elicits an enhanced TH1 and an enhanced TH2 response, i.e., an increase in the production of both IgG1 and IgG2a production relative to immunization without an adjuvant. Still more preferably, the composition comprising a combination of a TH1 and a TH2 adjuvant elicits an increased TH1 and/or an increased TH2 immune response relative to immunization with a single adjuvant (i.e., relative to immunization with a TH1 adjuvant alone or immunization with a TH2 adjuvant alone).

The immune response may be one or both of a TH1 immune response and a TH2 response. Preferably, immune response provides for one or both of an enhanced TH1 response and an enhanced TH2 response.

The enhanced immune response may be one or both of a systemic and a mucosal immune response. Preferably, the immune response provides for one or both of an enhanced systemic and an enhanced mucosal immune response. Preferably the mucosal immune response is a TH2 immune response. Preferably, the mucosal immune response includes an increase in the production of IgA.

The compositions of the invention may be prepared in various forms. For example, the compositions may be prepared as injectables, either as liquid solutions or suspensions. Solid forms suitable for solution in, or suspension in, liquid vehicles prior to injection can also be prepared (e.g. a lyophilised composition or a spray-freeze dried composition). The composition may be prepared for topical administration e.g. as an ointment, cream or powder. The composition may be prepared for oral administration e.g. as a tablet or capsule, as a spray, or as a syrup (optionally flavoured). The composition may be prepared for pulmonary administration e.g. as an inhaler, using a fine powder or a spray. The composition may be prepared as a suppository or pessary. The composition may be prepared as a solid dosage form for parenteral or needleless administration, for example intra-dermal administration. The composition may be prepared for nasal, aural or ocular administration e.g. as drops. The composition may be in kit form, designed such that a combined composition is reconstituted just prior to administration to a patient. Such kits may comprise one or more antigens in liquid form and one or more lyophilised antigens.

Where a composition is to be prepared extemporaneously prior to use (e.g. where a component is presented in lyophilised form) and is presented as a kit, the kit may comprise two vials, or it may comprise one ready-filled syringe and one vial, with the contents of the syringe being used to reactivate the contents of the vial prior to injection.

Immunogenic compositions used as vaccines comprise an immunologically effective amount of the pilus, as well as any other components, as needed. By ‘immunologically effective amount’, it is meant that the administration of that amount to an individual, either in a single dose or as part of a series, is effective for treatment or prevention. This amount varies depending upon the health and physical condition of the individual to be treated, age, the taxonomic group of individual to be treated (e.g. non-human primate, primate, etc.), the capacity of the individual's immune system to synthesise antibodies, the degree of protection desired, the formulation of the vaccine, the treating doctor's assessment of the medical situation, and other relevant factors. It is expected that the amount will fall in a relatively broad range that can be determined through routine trials. Examples of an immunologically effective amount are around 0.1 μg-10 μg pilus, for example 0.5 μg-10 μg pilus.

As mentioned above, a composition may include a temperature protective agent, and this component may be particularly useful in adjuvanted compositions (particularly those containing a mineral adjuvant, such as an aluminium salt). As described in reference 60, a liquid temperature protective agent may be added to an aqueous vaccine composition to lower its freezing point e.g. to reduce the freezing point to below 0° C. Thus the composition can be stored below 0° C., but above its freezing point, to inhibit thermal breakdown. The temperature protective agent also permits freezing of the composition while protecting mineral salt adjuvants against agglomeration or sedimentation after freezing and thawing, and may also protect the composition at elevated temperatures e.g. above 40° C. A starting aqueous vaccine and the liquid temperature protective agent may be mixed such that the liquid temperature protective agent forms from 1-80% by volume of the final mixture. Suitable temperature protective agents should be safe for human administration, readily miscible/soluble in water, and should not damage other components (e.g. antigen and adjuvant) in the composition. Examples include glycerin, propylene glycol, and/or polyethylene glycol (PEG). Suitable PEGs may have an average molecular weight ranging from 200-20,000 Da. In a preferred embodiment, the polyethylene glycol can have an average molecular weight of about 300 Da (PEG-300′).

Methods of Treatment, and Administration of the Vaccine

The invention also provides a method for raising an immune response in a mammal comprising the step of administering an effective amount of a composition of the invention, or a pilus of the invention. The immune response is preferably protective and preferably involves antibodies and/or cell-mediated immunity. The method may raise a booster response.

The invention also provides immunogenic combinations or compositions for use as a medicament e.g. for use in raising an immune response in a subject, such as a mammal.

The invention also provides the use of the pilus of the invention in the manufacture of a medicament for raising an immune response in a mammal.

By raising an immune response in the mammal by these uses and methods, the mammal can be protected against diseases caused by the bacteria from which the polypeptides in the pilus are derived. In particular, the mammal can be protected against disease caused by Streptococcal bacteria, including GAS, GBS and Streptococcus pneumoniae. The invention also provides a delivery device pre-filled with an immunogenic composition of the invention.

The mammal is preferably a human, a large veterinary mammal (e.g. horses, cattle, deer, goats, pigs) and/or a domestic pet (e.g. dogs, cats, gerbils, hamsters, guinea pigs, chinchillas). Most preferably, the mammal is a human, e.g. human patient. Where the vaccine is for prophylactic use, the human may be a child (e.g. a toddler or infant) or a teenager; where the vaccine is for therapeutic use, the human may be a teenager or an adult. A vaccine intended for children may also be administered to adults e.g. to assess safety, dosage, immunogenicity, etc. A mammal (e.g. human, e.g. a patient) may either be at risk from the disease themselves or may be a pregnant female, e.g. woman (‘maternal immunisation’). Vaccination of pregnant females may be advantageous as a means of providing antibody mediated passive protection to new born mammals. Maternal passive immunity is a type of naturally acquired passive immunity, and refers to antibody-mediated immunity conveyed to a fetus by its mother during pregnancy. Maternal antibodies (MatAb) are passed through the placenta to the fetus by an FcRn receptor on placental cells. This occurs around the third month of gestation. Particularly the antibodies are Immunoglobulin G (IgG) or Immunoglobulin A (IgA). IgGy antibody isotypes can pass through the placenta during pregancy. Passive immunity may also provided through the transfer of IgA antibodies found in breast milk that are transferred to the gut of the infant, protecting against bacterial infections, until the newborn can synthesize its own antibodies.

One way of checking efficacy of therapeutic treatment involves monitoring infection after administration of the compositions of the invention. One way of checking efficacy of prophylactic treatment involves monitoring immune responses, systemically (such as monitoring the level of IgG1 and IgG2a production) and/or mucosally (such as monitoring the level of IgA production), against the antigen(s) in the pilus of the invention after administration of the composition. Typically, antigen-specific serum antibody responses are determined post-immunisation but pre-challenge whereas antigen-specific mucosal antibody responses are determined post-immunisation and post-challenge.

Another way of assessing the immunogenicity of the compositions of the present invention is to express the proteins recombinantly for screening patient sera or mucosal secretions by immunoblot and/or microarrays. A positive reaction between the protein and the patient sample indicates that the patient has mounted an immune response to the protein in question. This method may also be used to identify immunodominant antigens and/or epitopes within antigens.

The efficacy of compositions of the invention can also be determined in vivo by challenging animal models of infection, e.g., guinea pigs or mice, with the vaccine compositions.

Compositions of the invention will generally be administered directly to a patient. Direct delivery may be accomplished by parenteral injection (e.g. subcutaneously, intraperitoneally, intravenously, intramuscularly, or to the interstitial space of a tissue), or mucosally, such as by rectal, oral (e.g. tablet, spray), vaginal, topical, transdermal or transcutaneous, intranasal, ocular, aural, pulmonary or other mucosal administration.

The invention may be used to elicit systemic and/or mucosal immunity, preferably to elicit an enhanced systemic and/or mucosal immunity.

Preferably the enhanced systemic and/or mucosal immunity is reflected in an enhanced TH1 and/or TH2 immune response. Preferably, the enhanced immune response includes an increase in the production of IgG1 and/or IgG2a and/or IgA.

Dosage can be by a single dose schedule or a multiple dose schedule. Multiple doses may be used in a primary immunisation schedule and/or in a booster immunisation schedule. In a multiple dose schedule the various doses may be given by the same or different routes e.g. a parenteral prime and mucosal boost, a mucosal prime and parenteral boost, etc. Multiple doses will typically be administered at least 1 week apart (e.g. about 2 weeks, about 3 weeks, about 4 weeks, about 6 weeks, about 8 weeks, about 10 weeks, about 12 weeks, about 16 weeks, etc.).

Vaccines prepared according to the invention may be used to treat both children and adults. Thus a human patient may be less than 1 year old, 1-5 years old, 5-15 years old, 15-55 years old, or at least 55 years old. Preferred patients for receiving the vaccines are the elderly (e.g. ≧50 years old, ≧60 years old, and preferably ≧65 years), the young (e.g. ≦5 years old), hospitalised patients, healthcare workers, armed service and military personnel, pregnant women, the chronically ill, or immunodeficient patients. The vaccines are not suitable solely for these groups, however, and may be used more generally in a population.

Vaccines produced by the invention may be administered to patients at substantially the same time as (e.g. during the same medical consultation or visit to a healthcare professional or vaccination centre) other vaccines e.g. at substantially the same time as a measles vaccine, a mumps vaccine, a rubella vaccine, a MMR vaccine, a varicella vaccine, a MMRV vaccine, a diphtheria vaccine, a tetanus vaccine, a pertussis vaccine, a DTP vaccine, a conjugated H. influenzae type b vaccine, an inactivated poliovirus vaccine, a hepatitis B virus vaccine, a meningococcal conjugate vaccine (such as a tetravalent A-C-W135-Y vaccine), a respiratory syncytial virus vaccine, etc.

Further Antigenic Components of Compositions of the Invention

The invention also provides compositions further comprising at least one further antigen.

In particular, the invention also provides a composition comprising a polypeptide of the invention and one or more of the following further antigens:
a saccharide antigen from N. meningitidis serogroup A, C, W135 and/or Y (preferably all four).
a saccharide or polypeptide antigen from Streptococcus pneumoniae [e.g. 61, 62, 63].
an antigen from hepatitis A virus, such as inactivated virus [e.g. 64, 65].
an antigen from hepatitis B virus, such as the surface and/or core antigens [e.g. 65, 66].
a diphtheria antigen, such as a diphtheria toxoid [e.g. chapter 3 of ref. 67] or the CRM₁₉₇ mutant [e.g. 68].
a tetanus antigen, such as a tetanus toxoid [e.g. chapter 4 of ref 67].
an antigen from Bordetella pertussis, such as pertussis holotoxin (PT) and filamentous haemagglutinin (FHA) from B. pertussis, optionally also in combination with pertactin and/or agglutinogens 2 and 3 [e.g. refs. 69 & 70].
a saccharide antigen from Haemophilus influenzae B [e.g. 71].
polio antigen(s) [e.g. 72, 73] such as IPV.
measles, mumps and/or rubella antigens [e.g. chapters 9, 10 & 11 of ref 67].
influenza antigen(s) [e.g. chapter 19 of ref. 67], such as the haemagglutinin and/or neuraminidase surface proteins.
an antigen from Moraxella catarrhalis [e.g. 74].
an protein antigen from Streptococcus agalactiae (group B streptococcus) [e.g. 75, 76].
a saccharide antigen from Streptococcus agalactiae (group B streptococcus).
an antigen from Streptococcus pyogenes (group A streptococcus) [e.g. 76, 77, 78].
an antigen from Staphylococcus aureus [e.g. 79].
an antigen from E. coli

The composition may comprise one or more of these further antigens.

Toxic protein antigens may be detoxified where necessary (e.g. detoxification of pertussis toxin by chemical and/or genetic means [70]).

Where a diphtheria antigen is included in the composition it is preferred also to include tetanus antigen and pertussis antigens. Similarly, where a tetanus antigen is included it is preferred also to include diphtheria and pertussis antigens. Similarly, where a pertussis antigen is included it is preferred also to include diphtheria and tetanus antigens. DTP combinations are thus preferred.

Saccharide antigens are preferably in the form of conjugates. Carrier proteins for the conjugates include diphtheria toxin, tetanus toxin, the N. meningitidis outer membrane protein [80], synthetic peptides [81,82], heat shock proteins [83,84], pertussis proteins [85,86], protein D from H. influenzae [87], cytokines [88], lymphokines [88], streptococcal proteins, hormones [88], growth factors [88], toxin A or B from C. difficile [89], iron-uptake proteins [90], etc. A preferred carrier protein is the CRM197 mutant of diphtheria toxin [91].

Antigens in the composition will typically be present at a concentration of at least 1 μg/ml each. In general, the concentration of any given antigen will be sufficient to elicit an immune response against that antigen.

As an alternative to using proteins antigens in the immunogenic compositions of the invention, nucleic acid (preferably DNA e.g. in the form of a plasmid) encoding the antigen may be used.

Antigens are preferably adsorbed to an aluminium salt.

Surprisingly, the Inventors have discovered that the pilin motif is not required for polymerisation by mutant sortases of the invention in contrast to the wild type sortases (from which the mutants are derived) wherein the presence of this motif is essential. In addition, mutant sortases of the invention can use different nucleophile/s to resolve the acyl-intermediate between the enzyme and the LPXTG-like sorting signal. In contrast, wild type sortases from which the mutant sortases are derived require the presence of a lysine residue. Mutant sortases of the invention are effective in vitro at catalysing transpeptidation reactions and forming polymers of GBS pilus proteins. Mutant sortases of the invention are further useful in a variety of protein engineering applications. The structural differences between the sortases of the present invention and other pilus-related sortases in gram positive bacteria may provide new functionality and enable new in vitro methods to be performed, or may allow polymerisation and ligation reactions to be performed more efficiently.

The mutant sortase enzymes of the invention are useful for performing ligation reactions between any moiety that comprises the LPXTG recognition motif (or those listed above) and any moiety that comprises an amino acid residue that can provide the nucleophile to complete the transpeptidation reaction. As shown in the Examples, mutant sortases of the invention are able to cleave and polymerise backbone proteins and ancillary proteins comprising the LPXTG motif. Previous work has demonstrated that bacterial sortases require only a single amino acid to provide the nucleophile to complete the transpeptidation reaction (Proft., Biotechnology Letters, 2010, 32:1-10; Popp et al., Current Protocols in Protein Science, 2009, 15, WO2010/087994).

In certain embodiments of the methods of the invention, either the first moiety or the second moiety in the ligation is a polypeptide and the other moiety is a protein or glycoprotein on the surface of a cell. The sortases of the invention can be used to attach polypeptides to proteins on the cell surface. This can be particularly useful for, for example, labelling specific proteins on the cell surface. In certain embodiments, the cell has been transfected to express the surface protein of interest with a LPXTG motif. This motif can then be targeted for ligation using a sortase of the invention. Alternatively, the protein label may comprise the motif.

Use of Sortases for Ligation of Substrates Other than Pilus Proteins

In other embodiments of the invention, mutant sortases of the invention are used to ligate proteins to a solid support and either the first moiety or the second moiety is a polypeptide and the other moiety comprises amino acids conjugated to a solid support. Such covalent attachment allows extensive washing to be carried out. In certain such embodiments, the protein comprises the LPXTG motif and the solid support has amino acids, such as lysine, conjugated to it. In certain embodiments the solid support is a bead, such as a polystyrene bead or gold bead or particle such as a nanoparticle.

Similarly, the methods of the invention allow circularisation of polypeptide chains. In such embodiments the first moiety and the second moiety are the N-terminus and C-terminus of a polypeptide chain, and ligation results in the formation of a circular polypeptide.

Bacterial sortases are also of significant interest for protein modification and engineering applications. Sortases promote pilin formation in vivo by catalysing a transpepditation reaction between backbone and ancillary proteins. Sortases recognise and cleave a recognition motif (for example, LPXTG) and form an amide linkage with a target protein. By utilising the recognition motif, a variety of protein engineering functions can be performed. Ligation reactions performed using sortases are flexible, efficient and require fewer steps than comparable chemical ligation techniques. Therefore, another object of the invention is to provide improved sortases for protein engineering applications. The techniques of Sortagging are known in the art.

In addition to the sortase mutants described above, other sortase enzymes may be used for ligation. For example, the sortases SrtC1 and SrtC2 from GBS pathogenicity island PI-2b.

The amino acid sequence of wild type SrtC1 from PI-2b is presented in SEQ ID NO:5. Particularly, SrtC1 as used in the methods of the invention does not comprise a signal peptide or N-terminal transmembrane domain (as in SEQ ID NO:98, SEQ ID NO:99 or SEQ ID NO:100). In certain preferred embodiments, SrtC1 as used in the methods of the invention comprises SEQ ID NO:101, which corresponds to the cloned soluble domain. SrtC1 comprising SEQ ID NO:101, which corresponds to the cloned soluble domain. In certain embodiments, SrtC1 may have a W55F mutation (as in SEQ ID NO:102). W55 may be important in regulating the activity of SrtC1, because it is located in the region that the canonical sortases lid motif is normally found in Streptococcal sortases. W55 may mimic the function of the lid found in other sortases. In certain embodiments, the SrtC1 as used in the methods of the invention may have a C188A mutation (as in SEQ ID NO:103). C188 may be a catalytic cysteine.

The amino acid sequence of wild type SrtC2 from PI-2b is presented in SEQ ID NO:105. In certain embodiments, the SrtC2 as used in the methods of the invention may have its cysteines substituted with alanines (as in SEQ ID NO:106). In certain embodiments of the invention, SrtC2 as used in the methods of the invention does not comprise a signal peptide or N-terminal transmembrane domain (as in SEQ ID NO:108 or SEQ ID NO:109). The skilled person is capable of identifying any signal peptide or N-terminal transmembrane domain.

PI-2b sortase C1 and sortase C2 enzymes for use with the invention may thus comprise or consist of an amino acid sequence: (a) having 70% or more identity (e.g. 75%, 80%, 85%, 90%, 91%, 92%, 93%, 94%, 95%, 96%, 97%, 98%, 99%, 99.5% or more) to a polypeptide having the amino acid sequence of any one of SEQ ID NOs:5, 98, 100, 101, 102, 103, 105, 106, 108 and 109; or (b) that is a fragment of at least ‘n’ consecutive amino acids of one of these sequences wherein ‘n’ is 100 or more (e.g. 120, 150, 170 or 190 or more). PI-2b sortase C1 and sortase C2 enzymes for use with the invention retain the ability to perform ligation and polymerisation reactions. The nucleotide sequences encoding SrtC1 and SrtC2 are provided in SEQ ID NO:104 and SEQ ID NO:107. Particular recognition motifs may include LPETGG, LPXTG, LPXT, LPKTG, LPATG, LPNTG, LPET, VPDT, IPQT, YPRR, LPMT, LAFT, LPQT, NSKT, NPQT, NAKT, NPQS, LPKT, LPIT, LPDT, SPKT, LAET, LAAT, LAET, LAST, LPLT, LSRT.

General

The practice of the present invention will employ, unless otherwise indicated, conventional methods of chemistry, biochemistry, molecular biology, immunology and pharmacology, within the skill of the art. Such techniques are explained fully in the literature. See, e.g., references 92-99, etc. The term “comprising” encompasses “including” as well as “consisting” e.g. a composition “comprising” X may consist exclusively of X or may include something additional e.g. X+Y.

The term “consisting essentially of” means that the composition, method or structure may include additional ingredients, steps and/or parts, but only if the additional ingredients, steps and/or parts do no materially alter the basic and novel characteristics of the claimed composition, method or structure. The term “consisting of” is generally taken to mean that the invention as claimed is limited to those elements specifically recited in the claim (and may include their equivalents, insofar as the doctrine of equivalents is applicable).

The term “about” in relation to a numerical value x means, for example, x+10%.

References to a percentage sequence identity between two amino acid sequences means that, when aligned, that percentage of amino acids are the same in comparing the two sequences. This alignment and the percent homology or sequence identity can be determined using software programs known in the art, for example those described in section 7.7.18 of ref. 100. A preferred alignment is determined by the Smith-Waterman homology search algorithm using an affine gap search with a gap open penalty of 12 and a gap extension penalty of 2, BLOSUM matrix of 62. The Smith-Waterman homology search algorithm is disclosed in ref. 101. The percent identity of a first polypeptide and a second polypeptide is generally determined by counting the number of matched positions between the first and second polypeptides and dividing that number by the total length of the shortest polypeptide followed by multiplying the resulting value by 100. For fragments of polypeptides this value is usually around 100% and therefore has little meaning. Therefore, in the context of fragments of the present invention, the term “proportion of reference polypeptide” (expressed as a percentage) is used. Proportion of reference polypeptide is calculated by counting the number of matched positions between the fragment and reference polypeptides and dividing that number by the total length of the reference polypeptide followed by multiplying the resulting value by 100. Particularly, fragments will comprise less than 90, 80, 70, 65, 60, 55, 50, 45, 40, 35, 30, 25 or less than 20% of the sequence of the reference polypeptide.

### MODES FOR CARRYING OUT THE INVENTION

### Example 1

### Functional Regulation of GBS SrtC1: A Single Mutation in the Lid Region Enhances BP Polymerization In Vitro

### Summary

Cell-surface pili are important virulence factors and promising vaccine candidates. Gram-positive bacteria elaborate pili via a sortase C-catalyzed transpeptidation mechanism from backbone and ancillary pilin substrates. For the covalent crosslinking of individual subunits, specific residues and/or motifs, such as the pilin motif and the conserved LPxTG sorting signal are absolutely necessary. Site-directed mutagenesis of GBS sortase C1 of Pi-2a (SrtC1) reveals the specific involvement of Tyr86 in the lid-regulatory site in the activation of recombinant SrtC1. This example shows that recombinant BP high molecular weight pili structures can be obtained in vitro using catalytic enzyme concentrations. This provides direct evidence of self-inhibition of sortase C enzymes by the presence of the lid and opens a field for studying pili assembly by using recombinant pili polymerized by a sortase-active mutant, reducing the necessity to purify high amount of wild type pili from pathogenic bacteria.

### BACKGROUND

Group B Streptococcus (GBS), or Streptococcus agalactiae, is the leading cause of life-threatening diseases in newborn and is also becoming a common cause of invasive disease in nonpregnant, elderly or immune-compromised adults [102]. Pili, long filamentous fibers protruding from bacterial surface, have been discovered in Gram-Positive pathogens as important virulence factors and potential vaccine candidates. From the analysis of the eight sequenced genomes of GBS, two genomic islands, each coding for three different pili, have been identified [103; 1]. Moreover, the srtA locus that encodes the ‘housekeeping’ sortase A (SrtA) is present in a different genome region in all analyzed GBS strains [103]. Each pilus genomic island codes for three LPXTG proteins: the backbone protein (BP) representing the main pilus subunit, and two ancillary proteins (AP1 and AP2). Moreover, each island encodes at least two class C sortases, each having specificity for one of the ancillary proteins [1; 104]. The crystal structures of several pilin related sortases, SrtC1-3 from S. pneumoniae [4], AcSrtC-1 from Actinomyces oris [105] and SrtC1 from S. suis (106) have been recently solved, with only the S. suis SrtC1 in an open active-site conformation Moreover, the crystal structure of S. pyogenes Spy 0129 has been solved, showing that it belongs to the class B sortase family, different to the other characterized pilin-specific sortases, which belong to class C. We have previously reported a structural and functional characterization of GBS SrtC1-2a. The crystal structure of the soluble core of GBS SrtC1-2a, containing its catalytic domain indicates that SrtC1 employs a catalytic triad composed of His157-Cys219-Arg228, essential for pilus fiber formation and covered by a loop, known as “lid”, which is dispensable for sortase activity in vivo [3]. Moreover, the crystal structure suggests that SrtC1 is folded as an auto-inactivated enzyme, by the presence of the lid that sterically blocks the active site. The function of the lid region in enzyme regulation and activity is still unclear, but is supposed to have a role in selecting the proper pilus proteins for polymerization. In this work we show, for the first time, efficient recombinant BP high molecular weight structures by using catalytic enzyme concentrations.

### Methods

### Bacterial Strains and Growth Conditions

GBS 515 strain and mutants were grown in Todd Hewitt Broth (THB) or in Trypticase soy agar (TSA) supplemented with 5% sheep blood at 37° C.

### Cloning, Expression and Purification of Recombinant Proteins

The proteins SrtC1₄₃₋₂₉₂, (SEQ ID NO:3 without signal and transmembrane domains), SrtC1_{Y86A} (SEQ ID NO:48) and SrtC1_{ΔLID} (SEQ ID NO:12) were expressed as His-MBP, TEV cleavable, fusion proteins and purified as previously described [3]. Recombinant BP₃₀₋₆₄₉, containing both the pilin motif and the sorting signal, was cloned in speedET vector and expressed as previously described [107], and BP_{K189A} was generated by PIPE site-directed mutagenesis using wild type BP₃₀₋₆₄₉. Recombinant BP₃₀₋₆₄₀, lacking the C-terminal LPxTG motif was cloned in speedET vector and expressed and purified as N terminal His-tag, TEV cleavable, fusion protein using the same protocol used for wild type BP.

Antisera

Antisera specific for the BP-2a and AP1-2a proteins were produced by immunizing CD1 mice with the purified recombinant proteins [107, 108].

Construction of Complementation Vectors and Site-Specific Mutagenesis

GBS knock-out (KO) mutant strain for BP was generated as previously reported [1]. For the generation of complementation vectors DNA fragments corresponding to wild type BP (SAL_{—}1486), gene was PCR amplified from GBS 515 genome and the product was cloned into the E. coli-streptococcal shuttle vector pAM401/gbs80P+T, previously described [11, 27] and containing the promoter and terminator regions of the gbs80 gene (TIGR annotation SAG_{—}0645). Site-directed mutagenesis of pAM_BP was performed using the PIPE (Polymerase Incomplete Primer Extension) method [19]. The complementation vectors pAM_BP_{ΔLPXTG} and pAM_BP_{K189A} were electroporated into the KO strain ΔBP. Complementation was confirmed by checking BP expression by Western Blotting.

Western Blotting Analysis

Mid-exponential phase bacterial cells were resuspended in 50 mM Tris-HCl containing 400 U of mutanolysin (Sigma-Aldrich) and COMPLETE protease inhibitors (Roche). The mixtures were then incubated at 37° C. for 1 h and cells lysed by three cycles of freeze-thawing. Cellular debris were removed by centrifugation and protein concentration was determined using BCA protein assay (Pierce, Rockford, Ill.). Total protein extracts (20 μg) or recombinant pili were resolved on 3-8% or 4-12% NuPAGE precast gels (Invitrogen) by SDS-PAGE and transferred to nitrocellulose. Membranes were probed with mouse antiserum directed against BP and AP1 proteins (1:1,000 dilution) followed by a rabbit anti-mouse horseradish peroxidase-conjugated secondary antibody (Dako, Glostrup, Denmark). Bands were then visualized using an Opti-4CN substrate kit (Bio-Rad).

Results

Lysine 189 in the Putative Pilin Motif and IPQTG Sorting Signal of BP-2a are Essential for Pilus Formation by Wild-Type Sortase C.

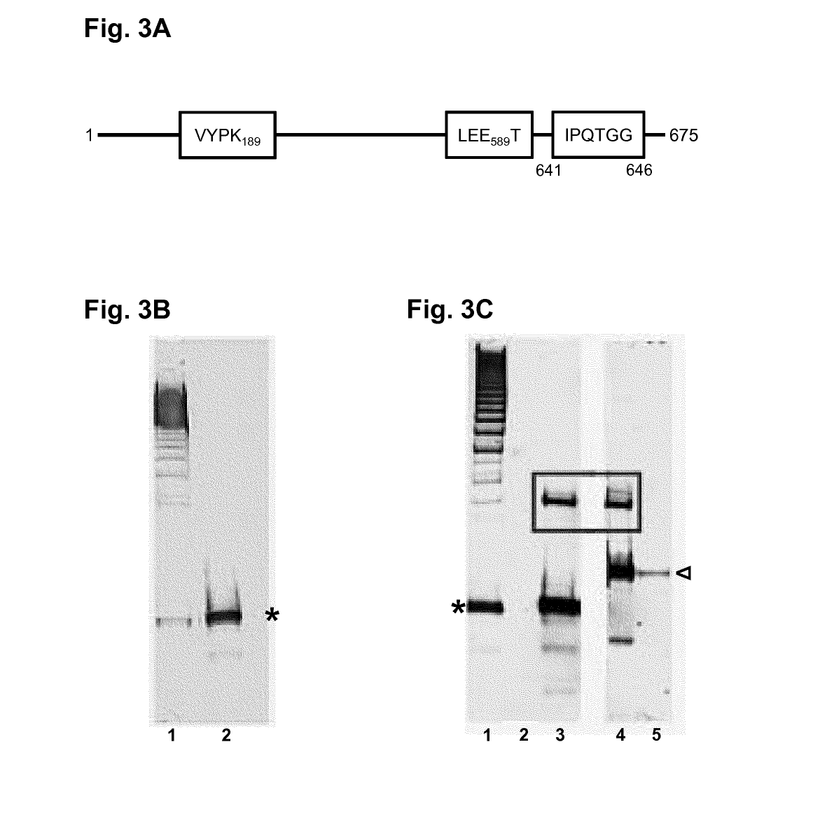

In the backbone protein of GBS pilus 2a, BP-2a (strain 515, TIGR annotation SAL_{—}1486) we identified a putative pilin motif containing a highly conserved lysine residue (Lys189) and the IPQTGG motif at residue 641-646 as the C terminus sorting motif (FIG. 3A). In order to investigate the specific contribution in pilus assembly of each residue/motif we used site-specific mutagenesis and complementation studies using the PIPE (Polymerase Incomplete Primer Extension) mutagenesis method to the vector pAM401 previously used in complementation studies of GBS knock-out (KO) mutant strains. As template for the introduction by PCR of specific mutations/deletions we used the complementation vector carrying the BP-2a gene (pAM_BP).

To evaluate the role of the Lys189 in the pilin motif and the IPQTGG motif in the cell wall sorting signal (CWSS) of BP-2a we generated a plasmid (pAM_B_{PK189A}) expressing a mutated backbone protein carrying a substitution of the pilin motif lysine residue with an alanine and a second plasmid (pAM_BP_{ΔIPQTG}) carrying the entire deletion of the IPQTG sorting signal. Both the K189 and the C terminus sorting signal of BP-2a were absolutely required for pilus polymerization and ancillary proteins incorporation into the high molecular weight structures (FIG. 3B). When the K189 was mutated into an alanine, only the monomer form of the BP could be identified, whereas when the sorting signal IPQTG was deleted in the BP, in addition to the monomeric form of BP a higher molecular weight band was also observed (FIG. 3C). Immunoblotting performed with antibodies raised against BP and AP1 showed that this higher molecular weight band, resistant to SDS treatment, contained both the backbone protein (BP) and the major ancillary protein (AP1) (FIG. 3C). The polymerization of the BP cannot occur as its sorting signal is deleted, but the pilin motif of the BP is still available for forming a covalent bond between the BP pilin motif and the AP1 sorting signal.

The LPXTG-Like Sorting Signal is Essential for the Transpeptidation Reaction Mediated In Vitro by the SrtC1_{Y86A} Mutant but the Pilin Motif is NOT.

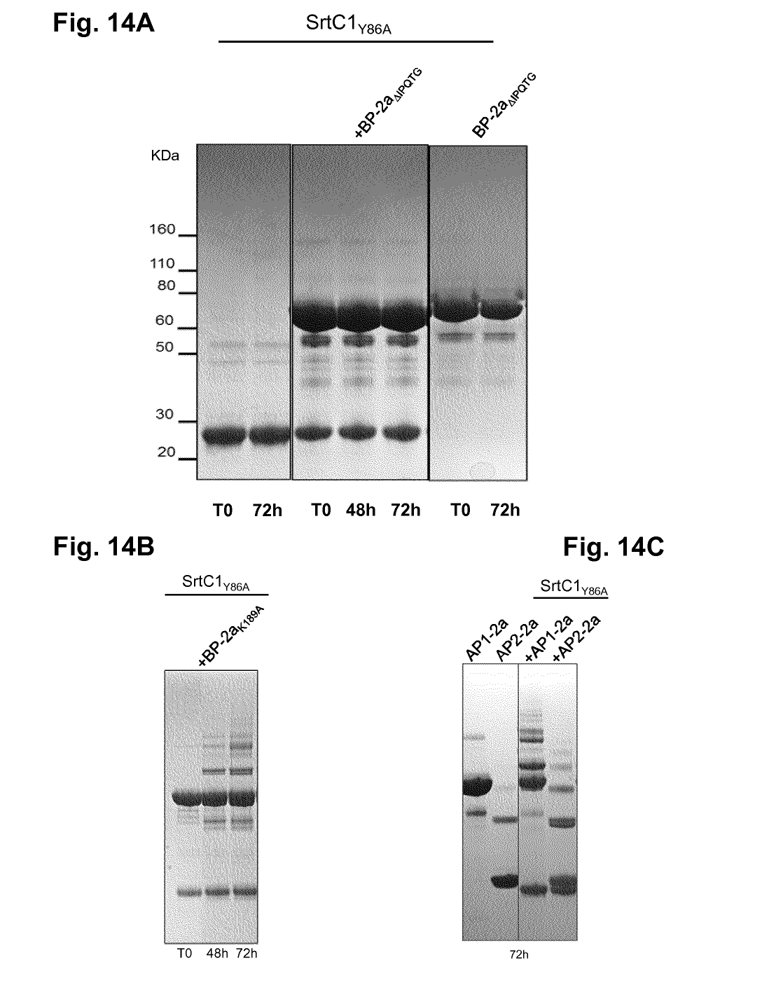

To investigate the specific contribution of the Lys189 in the pilin motif and the IPQTG sorting signal in the in vitro polymerization reaction, we expressed in E. coli and purified mutated forms of the BP-2a protein, BP_{ΔIPQTG} and BP_{K189A}, carrying the deletion of the IPQTG region and the substitution of the Lys189 with an alanine, respectively. After mixing the active SrtC1_{Y86A} with the recombinant BP_{ΔIPQTG} mutant, HMW polymers could not be detected, confirming that the polymerization reaction occurs through the cleavage of the sorting signal and the formation of the acyl-intermediate between SrtC1_{Y86A} and the IPQTG motif (FIG. 14A). On the contrary, in the reactions in which the active SrtC1_{Y86A} was incubated with BP_{K189A} HMW polymers could be observed, indicating that the Lys residue of the pilin motif (K189), differently from what happens in GBS, is not essential for in vitro polymerization (FIG. 14B). Moreover, when SrtC1_{Y86A} was mixed with recombinant forms of the ancillary proteins (AP1-2a and AP2-2a), that in vivo can be polymerized only in the presence of the BP-2a protein (data not shown), some HMW structures were formed (FIG. 14C). These data demonstrate that SrtC1_{Y86A} can use different nucleophile/s to resolve the acyl-intermediate between the enzyme and the LPXTG-like sorting signal. Therefore, since the pilin motif is not required, surprisingly this finding suggests that the mutant enzyme may be used in a broader range of reactions and is able to catalyse reactions with proteins to which an LPXTG motif has been added.

Wild-Type SrtC1-2a is not Able to Induce Recombinant BP Polymerization In Vitro.

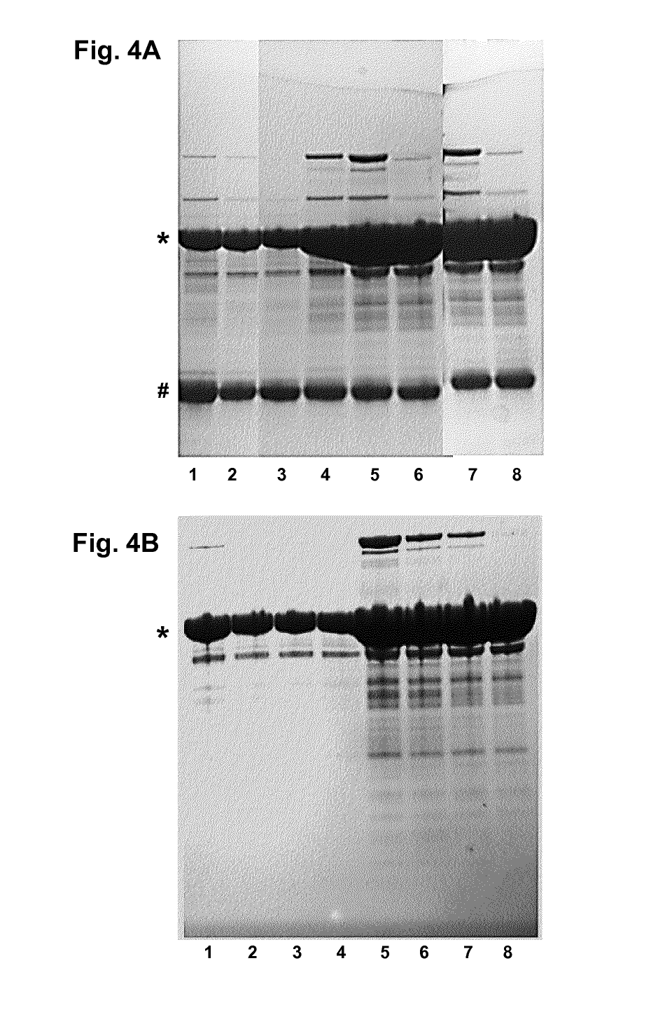

The presence of pili on GBS surface is characterized by a ladder of high-molecular-weight bands on SDS-PAGE by immunoblotting analysis of cell-wall preparations, in which GBS BP monomers are covalently linked forming the pilus backbone [1]. To test the hypothesis that it is the interaction with the backbone-protein substrate that induce the lid-open-active conformation of SrtC1, we tested the functional activity in vitro of recombinant SrtC1 (r-SrtC1) and recombinant backbone protein (r-BP) (107), by searching for a pattern of high-molecular-weight bands on gradient SDS gels. Recombinant GBS major pilin subunit BP carrying the pilin motif K189 and the C-terminal LPxTG recognition site, was mixed with WT SrtC1, at various ratios and incubated at 37° C. for different times reaching also the high enzyme amounts used for S. pneumoniae SrtC1 [4]. SDS-page analysis of these samples, however, showed no formation of high molecular weight bands that could represent pilus polymers (FIG. 4A), but only the formation of a complex compatible with the formation of a hetero-dimer formed by rSrtC1 and rBP, as previously described for S. pneumoniae [4] and a dimer BP-BP that is formed also in absence of SrtC1 (FIG. 4B).

BP High Molecular Weight Structures can be Assembled In Vitro by Recombinant SrtC1 Lid Mutant.

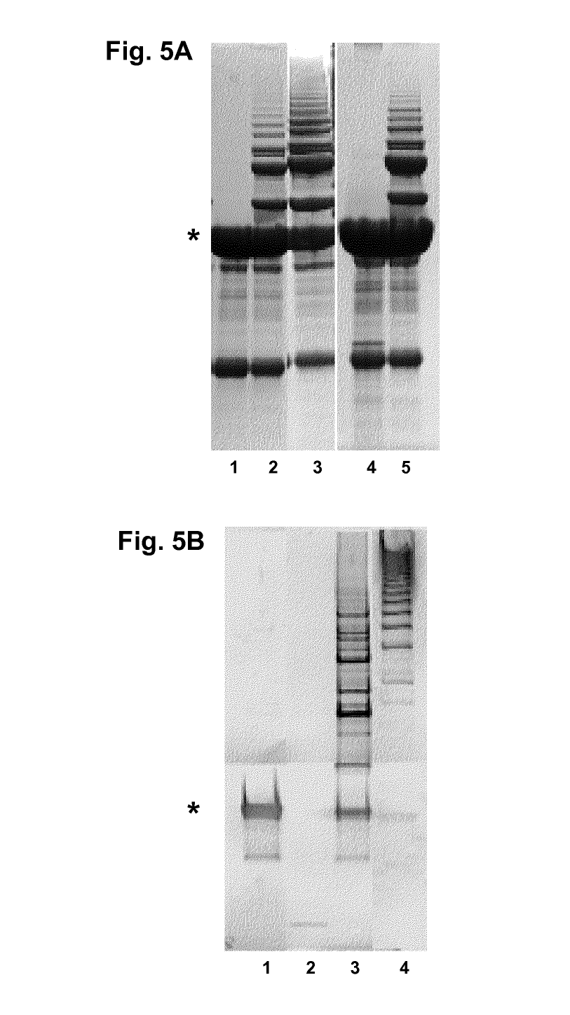

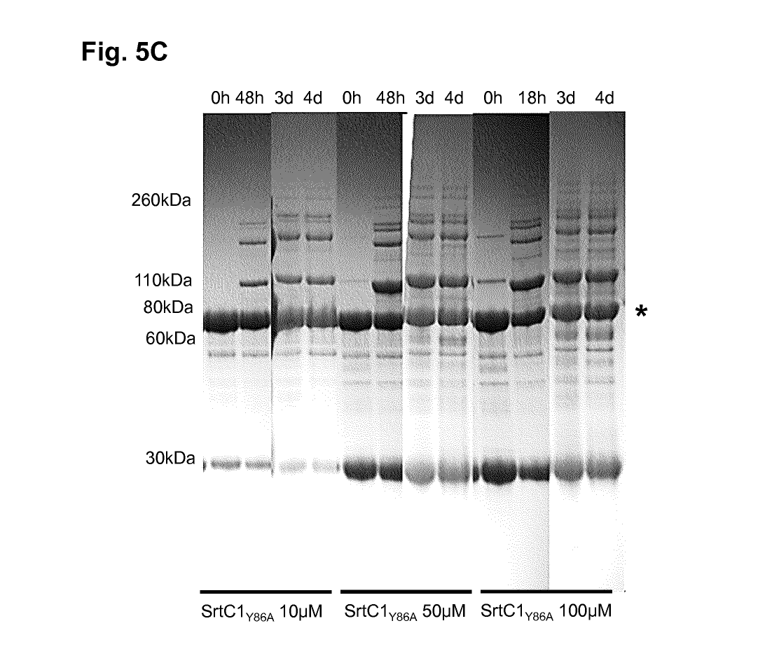

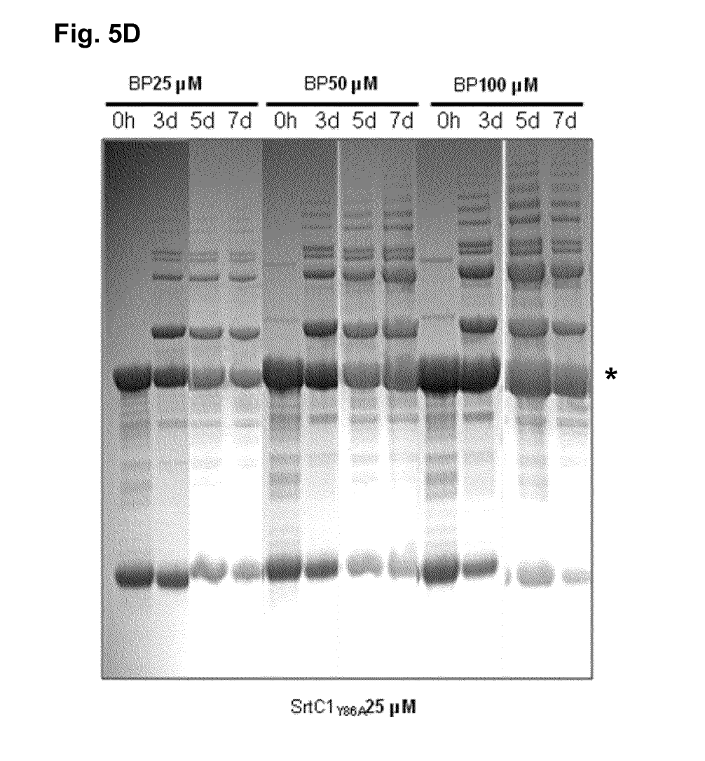

To confirm our hypothesis that the catalytic cysteine is locked by the aromatic ring of Tyr86, we performed the same experiment by mixing recombinant SrtC-1_{Y86A} [3], with recombinant purified BP and we tested the ability of this sortase mutant to polymerize GBS BP monomers. The typical pili pattern of bands with molecular weights above 260 kDa, visible by SDS-page, could be generated when monomeric r-BP was incubated with rSrtC1_{Y86A} (FIG. 5A). The reaction after 48 h was quenched and analyzed by Western Blotting using αBP antibodies, checking for the typical ladder of BP polymerization compared to wild type pili of GBS 515 strain (FIG. 5B).

As part of the BP monomer still remains unprocessed after 10 days of reaction, we tested if higher enzyme amounts could achieve a complete conversion of monomeric BP in polymeric structures.

We found that enzyme concentrations from 10 to 100 μM mixed with a fixed BP concentration did not change the rate of recombinant BP polymers formation (FIG. 5C).

Using a fixed enzyme concentration of 2504 for the polymerization reaction, varying concentration of monomeric BP were also tested (FIG. 5D).

BP High Molecular Weight Structures Formation In Vitro by Recombinant SrtC1y86A Mutant is Mediated by LPXTG and Pilin Motives.

To confirm that the polymerization of the BP occurs through the correct motives, the polymerization in vitro was tested by incubating r-BP_{ΔLPXTG} and r-BP_{K189A} with SrtC1_{Y86A} confirming that the polymerization occurs through the cleavage of the LPXTG sorting signal and the subsequent linking to the pilin motif of the next subunit.

Large-Scale Recombinant BP HMW Structures Production and Purification.

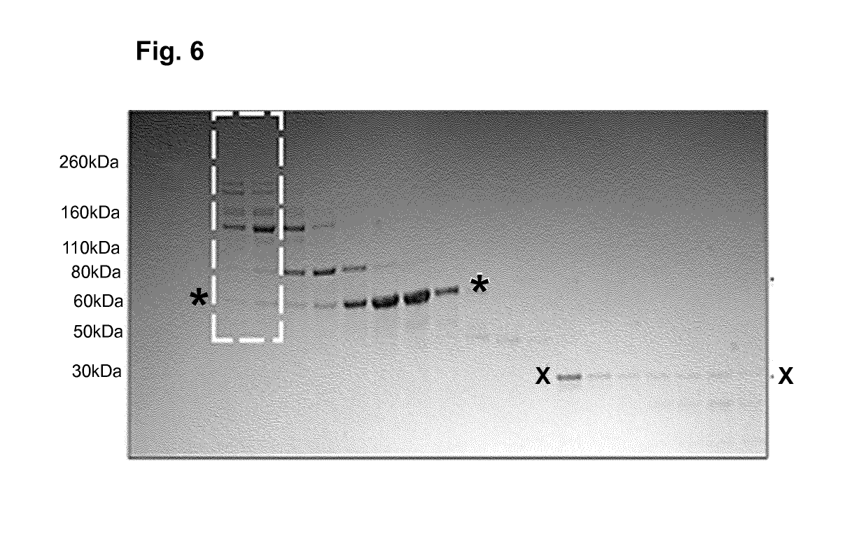

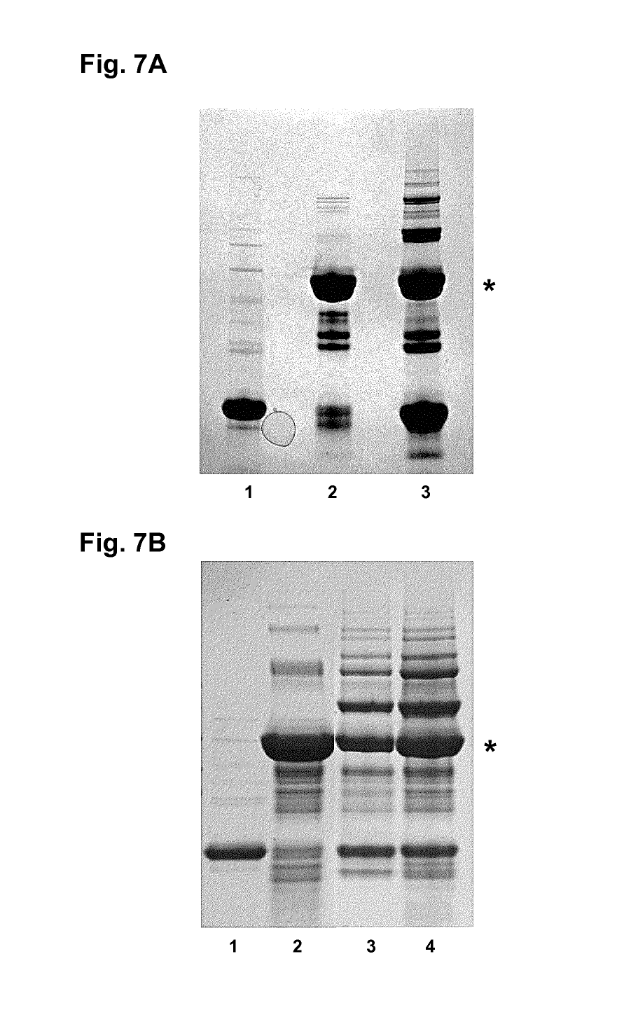

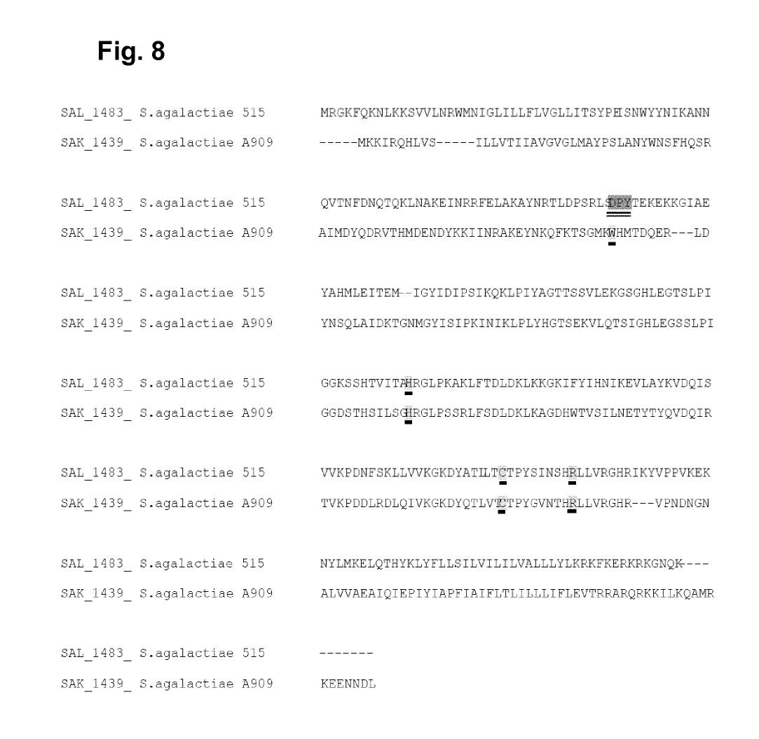

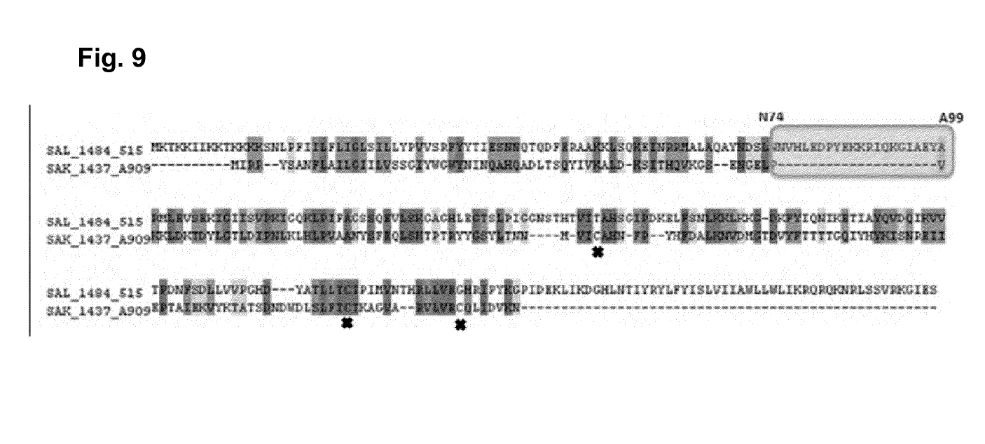

Pili purification from gram positive pathogens is very challenging and time consuming and allows the purification of low amounts of material only. As we could achieve BP polymerization in a 50 μl reaction volume, we tried to scale-up the production of recombinant pili production. We found that the best reaction conditions were achieved by using the enzyme at 25 μM and the BP at 100 μM. The reaction volume is also important, as using up to 100 μl of the reaction decreases the efficiency of BP polymerization. We performed 10 reactions using these concentrations of substrate and enzyme in 100 μl each, for a total amount of 6.5 mg of pure BP, and we incubated the reaction for 7 days in presence of reducing agent. After this time, the pool of the reactions (1 ml total) was separated by gel filtration. Two fractions, containing mostly high molecular weight pili, were isolated from the monomeric BP and SrtC1, and were quantified to contain 0.5 mg of pili (FIG. 6). FIG. 7 shows that mutant sortase enzymes polymerize pilus proteins from a variety of gram positive bacteria. SrtC1_{Y86A} (GBS sortase C1 of PI-2a) was incubated with backbone protein PI-1 of GBS (also referred to as GBS 80) (FIG. 7A) or with pilus protein from Streptococcus pneumoniae (also referred to as RrgB) (FIG. 7B).

### CONCLUSION

In Gram-positive bacteria the covalent association of pili requires the action of specific sortases. The pilus 2a biosynthesis in GBS is promoted by two sortase enzymes (SrtC-1 and SrtC-2) that polymerize the BP and display ancillary-proteins substrate specificity. Previously, we have shown that a triad composed of His, Cys and Arg residues is essential for SrtC-1 activity. Moreover, the crystal structure clearly indicates that GBS SrtC1 is auto-inhibited by the presence of the lid in the catalytic pocket. Recently, our group measured the catalytic activity for GBS SrtC1 by using a self-quenched fluorescent peptide mixed with recombinant GBS SrtC1 WT and lid mutants to monitor substrate cleavage, and we found that the lid-mutants are even more active than the WT. These data, in accordance with in vivo experiments with lid mutants, suggested that the activation of sortases C might occur by a conformational change that results in the movement of the lid away from the catalytic site that could be induced by the protein substrates.

Starting from these observations, we performed in vitro experiments using recombinant GBS SrtC1 WT and lid mutants mixed with recombinant backbone pilus protein and we observed that WT SrtC1 enzyme was not able to induce recombinant BP protein polymerization. Enzyme activation was achieved, in vitro, through a single mutation in the lid region of recombinant SrtC1-2a that enhances BP polymerization in vitro and recombinant pili formation. These experiments suggest that for SrtC, the mechanism behind recognition and polymerization of pilus subunits could not depend only on the interaction between the fimbrial shaft protein and the sortase, as the enzyme activation could not be achieved in vitro simply by mixing SrtC1WT with the BP. The experiments with the lid mutants indicate that the presence of the lid, and in particular of the Tyr86 in this loop, prevent BP polymerization. Our work provides the first direct evidence of self-inhibition of sortase C enzymes by the presence of the lid and opens a field for studying pili assembly by using recombinant pili polymerized by a sortase-active mutant, reducing the necessity to purify high amount of wild type pili from pathogenic bacteria. Moreover, the anchoring of many surface virulence factors on Gram-positive bacteria is mediated by sortase-activity and, therefore, these enzymes are attractive targets for the design of novel anti-infective therapeutics.

### Example 2

### Immunisation Studies Using In Vitro Polymerized Pili

The in vitro polymerized pili structures may be used in immunisation studies in mice. For example, 10 μg of purified recombinant pili may be mixed with an adjuvant (e.g. alum) and injected into mice in a final volume of 200 μl. This may be followed by one or more booster immunisations. The mice may then be analysed for an immune response to the pili structures. This immune response may be protective against the bacteria from which the monomeric pilus proteins were originally derived.

An immunisation study has been conducted in which mice were immunised with monomeric pili comprising GBS59 generated according to the methods of the invention in combination with alum, and the protective immune response was assessed following subsequent challenge with GBS. The results were compared to immunisation using a similar protocol with recombinant GBS59 not in pilus form and alum, the SrtCM1 (Y86A) mutant and alum, Crmla and alum. The results of the immunisation experiment are provided in Table 4 below.

TABLE 4
immunisation with GBS59 pili

| | Protective response to | Protective |
|---|---|---|
| Immunisation composition | challenge with GBS | response (%) |
| Recombinant pilus (GBS59) and | 70/70 | 100 |
| alum | | |
| Monomeric GBS59 515 and alum | 37/60 | 62 |
| SrtC1 (Y86A) mutant and alum | 4/80 | 5 |
| CRM Ia and alum | 40/40 | 100 |

These results show that the GBS59 pili generated using the mutant sortase C enzymes according to the methods of the invention are significantly more effective at generating a protective immune response to GBS than the recombinant monomeric protein and are equivalent to the use of CRM Ia.

### Example 3

### Polymerisation of BP-2a (GBS59) Variants In Vitro

We tested the ability of the sortase mutant to polymerize variants of GBS BP monomers of GBS59 corresponding to SEQ IDs: 74, 75, 76, 77, 78 and 79.

Bacterial Strains and Growth Conditions

The GBS strains used in this work were 2603 V/R (serotype V), 515 (Ia), CJB111 (V), H36B (serotype Ib), 5401 (II) and 3050 (II). Bacteria were grown at 37° C. in Todd Hewitt Broth (THB; Difco Laboratories) or in trypticase soy agar supplemented with 5% sheep blood.

Cloning, Expression, Purification of Recombinant Proteins and Antisera.

Genomic DNA was isolated by a standard protocol for gram-positive bacteria using a Nucleo Spin Tissue kit (Macherey-Nagel) according to the manufacturer's instructions. The full length recombinant BP-2a proteins, corresponding to 515, CJB111 and 2603 allelic variants (TIGR annotation SAL1486, SAM1372 and SAG1407, respectively), were produced as reported in Margarit et al, Journal of Infectious Diseases, 2009, 199: 108-115, whilst the full length H36B variant (TIGR annotation SAI_{—}1511) was cloned in pET24b+(Novagen) using strain H36B as source of DNA. Primers were designed to amplify the coding regions without the signal peptide and the 3′ terminal sequence starting from the LPXTG motif.

For recombinant protein expression, the cultures were maintained at 25° C. for 5h after induction with 1 mM IPTG for the pET clones or with 0.2% arabinose for the SpeedET clones. All recombinant proteins were purified by affinity chromatography and gel filtration. Briefly, cells were harvested by centrifugation and lysed in “lysis buffer”, containing 10 mM imidazole, 1 mg\ml lysozyme, 0.5 mg\ml DNAse and COMPLETE inhibitors cocktail (Roche) in PBS. The lysate was clarified by centrifugation and applied onto His-Trap HP column (Armesham Biosciences) pre-equilibrated in PBS containing 10 mM imidazole. Protein elution was performed using the same buffer containing 250 mM imidazole, after two wash steps using 20 mM and 50 mM imidazole buffers. The eluted proteins were then concentrated and loaded onto HiLoad 16/60 Superdex 75 (Amersham Biosciences) pre-equilibrated in PBS.

Antisera specific for each protein were produced by immunizing CD1 mice with the purified recombinant proteins as previously described (WO90/07936). Protein-specific immune responses (total Ig) in pooled sera were monitored by ELISA.

As before, we found that enzyme concentrations from 10 to 100 μM mixed with a fixed BP concentration did not change the rate of recombinant GBS59 polymer formation. GBS59 variant monomers were mixed at a 1:1:1:1:1:1 ratio. Using a fixed enzyme concentration of 25 μM for the polymerization reaction, varying concentrations of the mixture of variants of monomeric BP GBS59 were also tested.

In Vitro Polymerization with Three Variants of BP-2a (H36B, 515, CJB111):

BP-2a (variants H36B, 515, CJB111) concentrations: 35 μM each—105 μM tot.

SrtC1_{Y86A} concentration: 25 μM

Buffer: 25 mM Tris-HCl pH 7.5-100 mM NaCl-1 mM DTT

Total volume of reaction 100 μl

Incubation at 37° C. for 48 h

The typical pili pattern of bands with molecular weights above 260 kDa, visible by SDS-page, could be generated when the mixture of variants of monomeric r-BPs was incubated with rSrtC1_{Y86A} (FIG. 11). The reaction after 48 h was quenched and analyzed by Western Blotting using αBP antibodies, checking for the typical ladder of BP polymerization compared to wild type pili. Pili comprising each of the GBS59 variants were created and used for immunisation. Vaccination of mice following the procedures described above was successful in protecting against challenge with each of the three GBS strains. In contrast, mice vaccinated with only one variant form were only protected against challenge with that particular strain. Surprisingly, these artificial pili were more effective at generating a protective immune response to GBS than the recombinant monomeric protein

### Example 4

### In Vitro Polymerization with Two Type of Backbone Proteins (BP-2a+Pilus 1 BP (BP-1) and/or Pneumococcus RrgB)

Following the procedures outlined above, chimeric pili comprising backbone proteins from both Streptococcus agalactiae and Pneumococcus were prepared:

BP concentrations: 50 μM each—100 μM tot.

SrtC1_{Y86A} concentration: 25 μM

Buffer: 25 mM Tris-HCl pH 7.5-100 mM NaCl-1 mM DTT

Total volume of reaction 100 μl

Incubation at 37° C. for 48 h

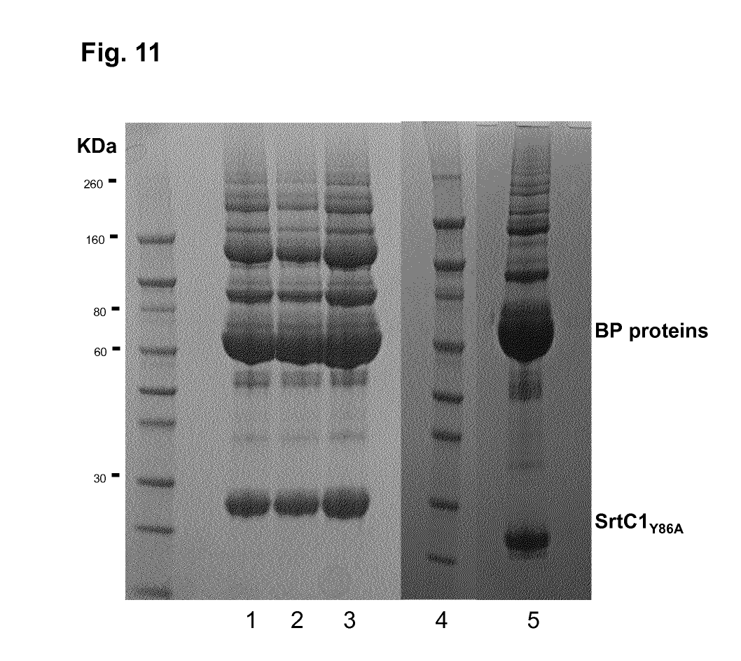

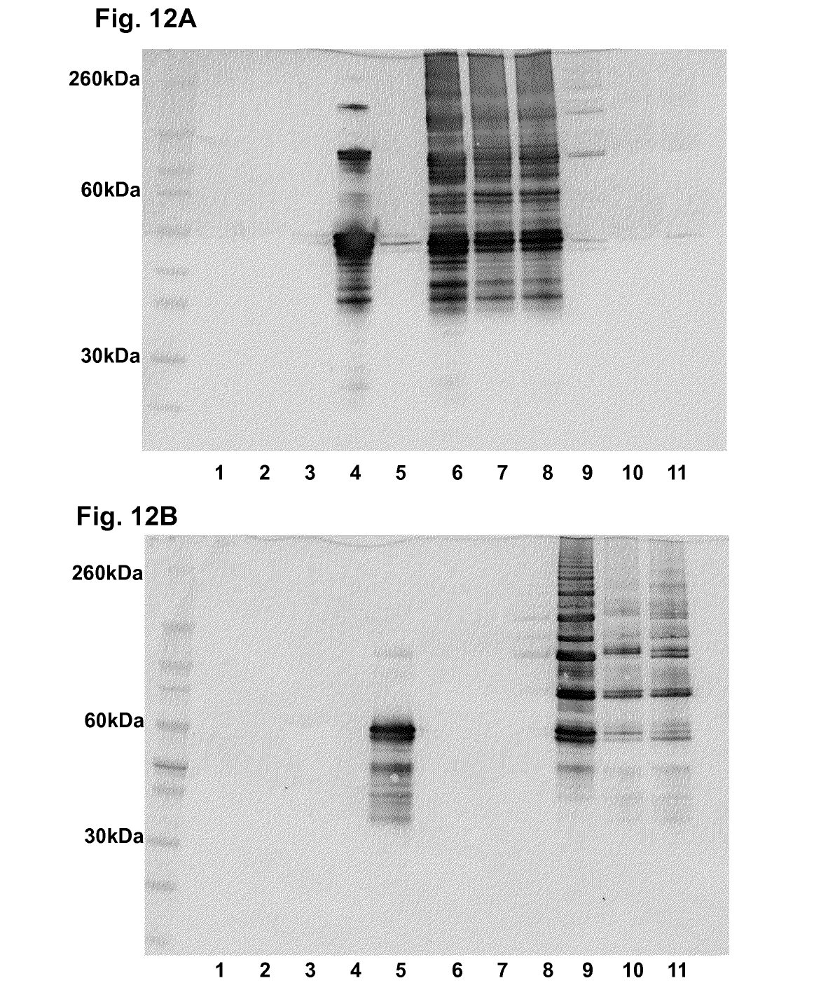

As shown in FIG. 12A and FIG. 12B, the presence of HMW bands demonstrates the ability of mutant sortase C enzymes to polymerise proteins from other strains/types of bacteria. Vaccination of mice following the procedures described above was successful in protecting against challenge with both Group B Streptococcus and Streptococcus pneumonia (data not shown). Sortases of the invention were also able to polymerise combinations of GBS67 and GBS59.

### Example 4

### Mutant SrtC can Polymerize GFP-IPQTG

The “IQTGGIGT” sequence was added at the C-terminus of the GFP protein DNA sequence using mutagenesis:

Primers Used:

GFP-lpxtg_F
attccacaaacaggtggtattggtacaTAACGCGACTTAATTAAACGG
GFP-lpxtg_R1
TGTACCAATACCACCTGTTTGTGGAATCTTGTACAGCTCGTCCATGCC

Mutagenesis DNA template: SpeedET vector+GFP

EGFP DNA sequence below (from pSpeedET):

CTTTAAGAAGGAGATATACATACCCATGGGATCTGATAAAATTCATCATC
ATCATCATCACGAAAACCTGTACTTCCAGGGCatggtgagcaagggcgag
gagctgttcaccggggtggtgcccatcctggtcgagctggacggcgacgt
aaacggccacaagttcagcgtgtccggcgagggcgagggcgatgccacct
acggcaagctgaccctgaagttcatctgcaccaccggcaagctgcccgtg
ccctggcccaccctcgtgaccaccctgacctacggcgtgcagtgcttcag
ccgctaccccgaccacatgaagcagcacgacttcttcaagtccgccatgc
ccgaaggctacgtccaggagcgcaccatcttcttcaaggacgacggcaac
tacaagacccgcgccgaggtgaagttcgagggcgacaccctggtgaaccg
catcgagctgaagggcatcgacttcaaggaggacggcaacatcctggggc
acaagctggagtacaactacaacagccacaacgtctatatcatggccgac
aagcagaagaacggcatcaaggtgaacttcaagatccgccacaacatcga
ggacggcagcgtgcagctcgccgaccactaccagcagaacacccccatcg
gcgacggccccgtgctgctgcccgacaaccactacctgagcacccagtcc
gccctgagcaaagaccccaacgagaagcgcgatcacatggtcctgctgga
gttcgtgaccgccgccgggatcactctcggcatggacgagctgtacaagT
AACGCGACTTAATTAAACGGTCTCCAGCTTGGCTGTTTTGGCGGATGAGA
GAAGATTTTCAGCCTGATACAGATTAAATC

EGFP amino acid sequence below (from pSpeedET):

MGSDKIHHHHHHENLYFQGMVSKGEELFTGVVPILVELDGDVNGHKFSVS
GEGEGDATYGKLTLKFICTTGKLPVPWPTLVTTLTYGVQCFSRYPDHMKQ
HDFFKSAMPEGYVQERTIFFKDDGNYKTRAEVKFEGDTLVNRIELKGIDF
KEDGNILGHKLEYNYNSHNVYIMADKQKNGIKVNFKIRHNIEDGSVQLAD
HYQQNTPIGDGPVLLPDNHYLSTQSALSKDPNEKRDHMVLLEFVTAAGIT
LGMDELYK

Nucleic acid sequence after mutagenesis:

CTTTAAGAAGGAGATATACATACCCATGGGATCTGATAAAATTCATCATC
ATCATCATCACGAAAACCTGTACTTCCAGGGCatggtgagcaagggcgag
gagctgttcaccggggtggtgcccatcctggtcgagctggacggcgacgt
aaacggccacaagttcagcgtgtccggcgagggcgagggcgatgccacct
acggcaagctgaccctgaagttcatctgcaccaccggcaagctgcccgtg
ccctggcccaccctcgtgaccaccctgacctacggcgtgcagtgcttcag
ccgctaccccgaccacatgaagcagcacgacttcttcaagtccgccatgc
ccgaaggctacgtccaggagcgcaccatcttcttcaaggacgacggcaac
tacaagacccgcgccgaggtgaagttcgagggcgacaccctggtgaaccg
catcgagctgaagggcatcgacttcaaggaggacggcaacatcctggggc
acaagctggagtacaactacaacagccacaacgtctatatcatggccgac
aagcagaagaacggcatcaaggtgaacttcaagatccgccacaacatcga
ggacggcagcgtgcagctcgccgaccactaccagcagaacacccccatcg
gcgacggccccgtgctgctgcccgacaaccactacctgagcacccagtcc
gccctgagcaaagaccccaacgagaagcgcgatcacatggtcctgctgga
gttcgtgaccgccgccgggatcactctcggcatggacgagctgtacaaga
ttccacaaacaggtggtattggtacaTAACGCGACTTAATTAAACGGTCT
CCAGCTTGGCTGTTTTGGCGGATGAGAGAAGATTTTCAGCCTGATACAGA
TTAAATC

Amino acid sequence after mutagenesis:

MGSDKIHHHHHHENLYFQGMVSKGEELFTGVVPILVELDGDVNGHKFSVS
GEGEGDATYGKLTLKFICTTGKLPVPWPTLVTTLTYGVQCFSRYPDHMKQ
HDFFKSAMPEGYVQERTIFFKDDGNYKTRAEVKFEGDTLVNRIELKGIDF
KEDGNILGHKLEYNYNSHNVYIMADKQKNGIKVNFKIRHNIEDGSVQLAD
HYQQNTPIGDGPVLLPDNHYLSTQSALSKDPNEKRDHMVLLEFVTAAGIT
LGMDELYKIPQTGGIGT-

Production of Recombinant “GFP-IQTGGIGT”: Expression and Purification

GFP-IPQTGGIGT Expression in HK100 in LB+kanamicine 30 ug\ml using biosilta media at 30° C., induction with arabinose 0.15% final. Purification: standard IMAC

GFP-IQTGGIGT and SrtC1Y86A Polymerization Reaction:

mix 25 uM SrtC1Y86A with 25-50 or 100 uM GFP-IPQTGGIGT in buffer 25 mM Tris pH7.5, 150 mM NaCl, DTT1 mM for 72h at 37° C. in termomixer. Reaction volume 50 ul.

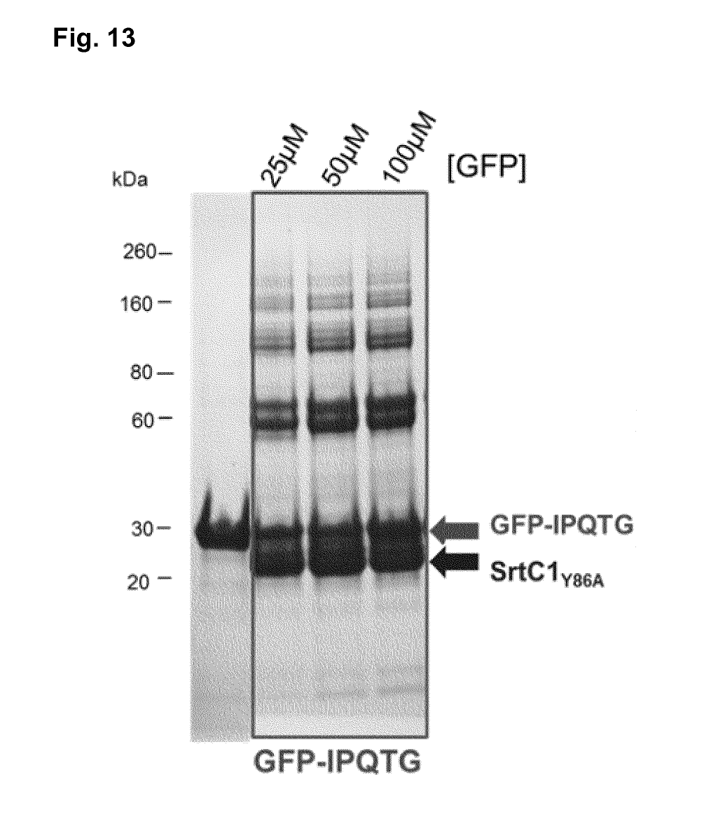

As shown in FIG. 13, the SrtC1_{Y86A} mutant was able to polymerise GFP-IPQTG.

### Example 5

### Recombinant PI-2b SrtC1 and SrtC2 Proteins are Active In Vitro and are Able to Cleave Fluorescent Peptides Carrying the LPXTG-Like Motif of Pilus Proteins

Full-length SrtC1 and C2 were cloned (using strain COH1 as template) in fusion with a His-MBP-tag. Recombinant enzymes were then expressed in E. coli and purified with IMAC or IMAC and MBP-trap column. FRET assays with purified sortases were carried out using synthetic fluorescent peptides carrying the LPXTG sorting motif of PI-1 backbone protein and of PI-1 minor ancillary protein in order to assess the catalytic activity. The PI-2b SrtC1 and SrtC2 enzymes are able to cleave the fluorescent peptides. These data demonstrate that the PI-2b SrtC1 and SrtC2 enzymes are active in vitro and are suitable for use in ligating and polymerising proteins.

The following protocols and conditions were used:

Purification of SrtC1 Enzyme—IMAC
3 litre culture of Rosetta cells expressing the SrtC1-MBP-His construct
pellets collected and lysed
10 mM Imidazole added to 30 ml lysate
column: 5 ml and 4 flow (approximately 5 ml/min)
lysate loaded and through flow collected
washed with 15 ml of buffer with 10 mM Imidazole (the first 3 ml are the dead volume of the column)
washed with 15 ml of buffer with 20 mM Imidazole
eluted with 300 mM Imidazole buffer 10 ml
1 mM DTT added
protein concentrated with amicon at 6000 rpm to 4° C. for 20 minutes
the final protein concentration was 1.78 mg/ml

Purification of SrtC2 Enzyme—IMAC
3 litre culture of Rosetta cells expressing the SrtC2-MBP-His construct
2 columns (30 ml of pellets with cell lysate+20 ml of Buffer 10 mM) 50 ml FT
pre-flushed with 20 ml of buffer 10 mM
washed with 50 ml of buffer 10 mM
washed with 150 ml of buffer 20 mM
elution buffer 300 mM: 5 ml dead volume, 10 ml elute2+20 μl DTT 1M, 10 ml elute3
elutes 1 and 3 were combined, whereas 10 ml of 300 mM NaCl, 50 mM and Tris pH8 were added to 10 ml of elute 2

Purification of SrtC2 Enzyme—MBP-Trap
2 columns were used MBP-trap
the column was washed with 50 ml of buffer with maltose (Tris 50 mM, 150 mM NaCl, pH8 Maltose 100 mM)
washed with 50 ml Urea 8 m pH8-Tris 50 mM
washed with distilled water (80 ml)
balanced with 25 ml of Tris buffer 50 mM pH8, 300 mM NaCl diluted with water 1:2
elute2 loaded
elutes 1+3 loaded
washed
eluted with buffer containing maltose

FRET Analysis

Closed plate with termofluor plastic.

1) 50 μl buffer (300 mM NaCl+50 mM Tris pH8)+50 μl 11515+1 μl BP peptide

2) 50 μl buffer (300 mM NaCl+50 mM Tris pH8)+50 μl 11515+1 μl AP2 peptide

3) 100 μl 11515+1 μl BP peptide

4) 100 μl 11515+1 μl AP2 peptide

5) 100 μl 1 elution buffer (300 mM Imidazole)+1 μl BP peptide

6) 100 μl 1 elution buffer (300 mM Imidazole)+1 μl AP2 peptide

We used 200 μl [1.78 mg/ml] of concentrated protein+2 μl LPXTG peptide of BP and AP2, and as control used the elution buffer 300 mM Imidazole instead of protein.

Tecan plate reader—300 cycles with a measurement every 10 minutes, temperature [34-37.5° C.°] with 37° C. for optimum and wavelength [400 nm-600 nm] have been obtained with maximum absorption provided to 500 nm.

### Example 6

### SrtC1 is Effective for Polymerising BP

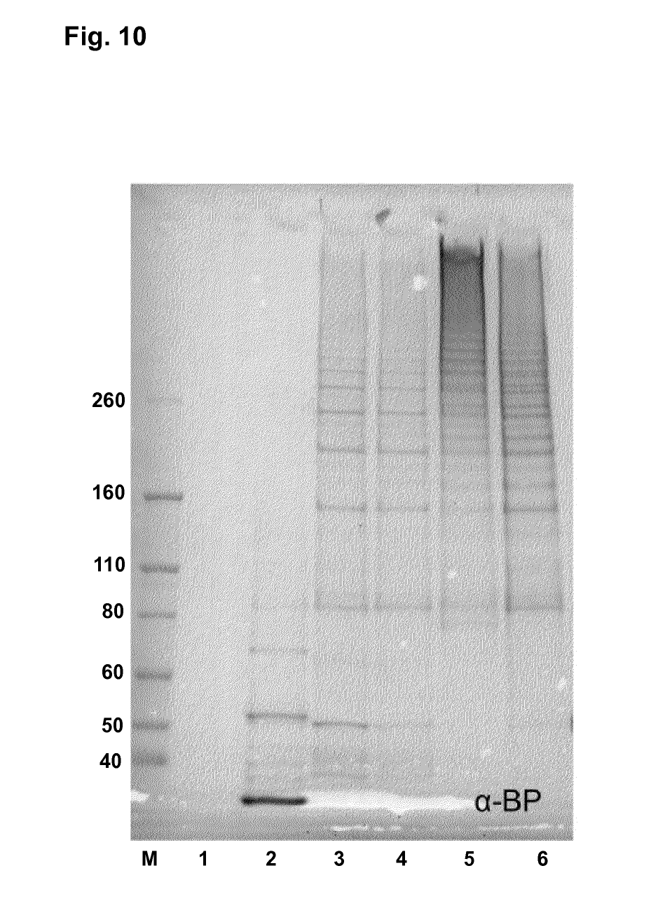

The activity of SrtC1 was further assayed by using a mutant GBS strain that does not express any pili (515Δ2a). This strain was transformed with complementation vectors PAMp80/t80 carrying genes coding for BP alone or BP with PI-2b SrtC1. The ability of the complementation vectors to restore pili polymerisation was analysed by western blot. As shown in FIG. 10, transfection with BP alone did not result in any polymerisation. However, transfection with BP and SrtC1 resulted in the formation of high molecular weight polymers. Strain A909, which expresses pilus 2b, was used as a positive control.

FIG. 10 provides a western blot of the membrane preparation from the 515Δ2a mutant strain and from the wild type A909 strain complemented by a plasmid containing SrtC1 and BP genes or BP gene alone. Antibodies against SrtC1 were used. Expected signals at 30 kDa confirm the expression and correct localization of SrtC1.

These data demonstrate that PI-2b SrtC1 is effective at polymerising pili.

The following protocols and conditions were used:

Electroporation

100 μl of the A909 and 515Δ2a strains were transformed with 3/7 μl of Spb1 (BP-PI-2b) or Spb1+SrtC1 (PI-2b).

Inoculation

In 10 ml THB+clm glycerol.

Cells were pelleted and washed with 25 ml PBS
940 μl TRIS pH 6.8, 50 mM+60μ (10 U/μl)
2 hours at 37° C., shaking

Gel and Western Blot GBS Extracts
10 extracts centrifuged for 10 minutes at maximum speed
30 μl 1 supernatant+15 μl 1 of LDS+5 μl of reducing
pellets were resuspended in 2% buffer TRIS 50 mM SDS pH8 300 mm NaCl
western blot and membrane washed
washed with water
2 hours stirring with milk 5%
rinsed with PBS
on every membrane 5 ml of milk 1%+antibody (anti-Spb1 on culture supernatants and anti SrtC1 on pellets)
left over night in with shaking in cold room
washed with 10 ml of PBS-Tween 0.05% for 10 minutes 3 times
washed with PBS for 5 minutes
20 ml of 1% milk+P161 anti-mouse antibody and left for 1 hour with stirring
washed with PBS
development solution prepared (10 ml=9 ml water+1 ml diluent+200 μl sample substrate+)—5 ml per membrane

SEQUENCES

| SEQ | |
|---|---|
| ID | |
| NO: | Polypeptide Sequence |
| 1 | MGQKSKISLATNIRIWIFRLIFLAGFLVLAFPIVSQVMYFQASHANINAFKEAVTKIDRVEINRRLE |
| | LAYAYNASIAGAKTNGEYPALKDPYSAEQKQAGVVEYARMLEVKEQIGHVIIPRINQDIPIYAGSAE |
| | ENLQRGVGHLEGTSLPVGGESTHAVLTAHRGLPTAKLFTNLDKVTVGDRFYIEHIGGKIAYQVDQIK |
| | VIAPDQLEDLYVIQGEDHVTLLTCTPYMINSHRLLVRGKRIPYVEKTVQKDSKTFRQQQYLTYAMWV |
| | VVGLILLSLLIWFKKTKQKKRRKNEKAASQNSHNNSK |
| 2 | MKKRLVKIVTIIRNNKIRTLIFVMGSLILLFPIVSQVSYYLASHQNINQFKREVAKIDTNTVERRIA |
| | LANAYNETLSRNPLLIDPFTSKQKEGLREYARMLEVHEQIGHVAIPSIGVDIPIYAGTSETVLQKGS |
| | GHLEGTSLPVGGLSTHSVLTAHRGLPTARLFTDLNKVKKGQIFYVTNIKETLAYKVVSIKVVDPTAL |
| | SEVKIVNGKDYITLLTCTPYMINSHRLLVKGERIPYDSTEAEKHKEQTVQDYRLSLVLKILLVLLIG |
| | LFIVIMMRRWMQHRQ |
| 3 | MKTKKIIKKTKKKKSNLPFIILFLIGLSILLYPVVSRFYYTIESNNQTQDFERAAKKLSQKEINRRM |
| | ALAQAYNDSLNNVHLEDPYEKKRIQKGIAEYARMLEVSEKIGIISVPKIGQKLPIFAGSSQEVLSKG |
| | AGHLEGTSLPIGGNSTHTVITAHSGIPDKELFSNLKKLKKGDKFYIQNIKETIAYQVDQIKVVTPDN |
| | FSDLLVVPGHDYATLLTCTPIMVNTHRLLVRGHRIPYKGPIDEKLIKDGHLNTIYRYLFYISLVIIA |
| | WLLWLIKRQRQKNRLSSVRKGIES |
| 4 | MRGKFQKNLKKSVVLNRWMNIGLILLFLVGLLITSYPFISNWYYNIKANNQVTNFDNQTQKLNAKEI |
| | NRRFELAKAYNRTLDPSRLSDPYTEKEKKGIAEYAHMLEITEMIGYIDIPSIKQKLPIYAGTTSSVL |
| | EKGSGHLEGTSLPIGGKSSHTVITAHRGLPKAKLFTDLDKLKKGKIFYIHNIKEVLAYKVDQISVVK |
| | PDNFSKLLVVKGKDYATLLTCTPYSINSHRLLVRGHRIKYVPPVKEKNYLMKELQTHYKLYFLLSIL |
| | VILILVALLLYLKRKFKERKRKGNQK |
| 5 | MAYPSLANYWNSFHQSRAIMDYQDRVTHMDENDYKKIINRAKEYNKQFKTSGMKWHMTSQERLDYNS |
| | QLAIDKTGNMGYISIPKINIKLPLYHGTSEKVLQTSIGHLEGSSLPIGGDSTHSILSGHRGLPSSRL |
| | FSDLDKLKVGDHWTVSILNETYTYQVDQIRTVKPDDLRDLQIVKGKDYQTLVTCTPYGVNTHRLLVR |
| | GHRVPNDNGNALVVAEAIQIEPIYIAPFIAIFLTLILLLISLEVTRRARQRKKILKQAMRKEENNDL |
| 6 | MAVMAYPLVSRLYYRVESNQQIADFDKEKATLDEADIDERMKLAQAFNDSLNNVVSGDPWSEEMKKK |
| | GRAEYARMLEIHERMGHVEIPVIDVDLPVYAGTAEEVLQQGAGHLEGTSLPIGGNSTHAVITAHTGL |
| | PTAKMFTDLTKLKVGDKFYVHNIKEVMAYQVDQVKVIEPTNFDDLLIVPGHDYVTLLTCTPYMINTH |
| | RLLVRGHRIPYVAEVEEEFIAANKLSHLYRYLFYVAVGLIVILLWIIRRLRKKKKQPEKALKALKAA |
| | RKEVKVEDGQQ |
| 7 | MSRYYYRIESNEVIKEFDETVSQMDKAELEERWRLAQAFNATLKPSEILDPFTEQEKKKGVSEYANM |
| | LKVHERIGYVEIPAIDQEIPMYVGTSEDILQKGAGLLEGASLPVGGENTHTVITAHRGLPTAELFSQ |
| | LDKMKKGDIFYLHVLDQVLAYQVDQIVTVEPNDFEPVLIQHGEDYATLLTCTPYMINSHRLLVRGKR |
| | IPYTAPIAERNRAVRERGQFWLWLLLGAMAVILLLLYRVYRNRRIVKGLEKQLEGRHVKD |
| 8 | MSRTKLRALLGYLLMLVACLIPIYCFGQMVLQSLGQVKGHATFVKSMTTEMYQEQQNHSLAYNQRLA |
| | SQNRIVDPFLAEGYEVNYQVSDDPDAVYGYLSIPSLEIMEPVYLGADYHHLGMGLAHVDGTPLPLDG |
| | TGIRSVIAGHRAEPSHVFFRHLDQLKVGDALYYDNGQEIVEYQMMDTEIILPSEWEKLESVSSKNIM |
| | TLITCDPIPTFNKRLLVNFERVAVYQKSDPQTAAVARVAFTKEGQSVSRVATSQWLYRGLVVLAFLG |
| | ILFVLWKLARLLRGK |
| 9 | MECYRDRQLLSTYHKQVTQKKPSEMEEVWQKAKAYNARLGIQPVPDAFSFRDGIHDKNYESLLQIEN |
| | NDIMGYVEVPSIKVTLPIYHYTTDEVLTKGAGHLFGSALPVGGDGTHTVISAHRGLPSAEMFTNLNL |
| | VKKGDTFYFRVLNKVLAYKVDQILTVEPDQVTSLSGVMGKDYATLVTCTPYGVNTKRLLVRGHRIAY |
| | HYKKYQQAKKAMKLVDKSRMWAEVVCAAFGVVIAIILVFMYSRVSAKKSK |
| 10 | IVSQVMYFQASHANINAFKEAVTKIDRVEINRRLELAYAYNASIAGAKTNGEYEYARMLEVKEQIGH |
| | VIIPRINQDIPIYAGSAEENLQRGVGHLEGTSLPVGGESTHAVLTAHRGLPTAKLFTNLDKVTVGDR |
| | FYIEHIGGKIAYQVDQIKVIAPDQLEDLYVIQGEDHVTLLTCTPYMINSHRLLVRGKRIPYVEKTVQ |
| | KDSKTFRQQQYLTYAMWVVVGLILLSLLIWFKKTKQKKRRKNEKAASQNSHNNSK |
| 11 | ASHQNINQFKREVAKIDTNTVERRIALANAYNETLSREYARMLEVHEQIGHVAIPSIGVDIPIYAGT |
| | SETVLQKGSGHLEGTSLPVGGLSTHSVLTAHRGLPTARLFTDLNKVKKGQIFYVTNIKETLAYKVVS |
| | IKVVDPTALSEVKIVNGKDYITLLTCTPYMINSHRLLVKGERIPYDSTEAEKHKEQTVQDYRLSLVL |
| | KILLVLLIGLFIVIMMRRWMQHRQ |
| 12 | ESNNQTQDFERAAKKLSQKEINRRMALAQAYNDSLNNVEYARMLEVSEKIGIISVPKIGQKLPIFAG |
| | SSQEVLSKGAGHLEGTSLPIGGNSTHTVITAHSGIPDKELFSNLKKLKKGDKFYIQNIKETIAYQVD |
| | QIKVVTPDNFSDLLVVPGHDYATLLTCTPIMVNTHRLLVRGHRIPYKGPIDEKLIKDGHLNTIYRYL |
| | FYISLVIIAWLLWLIKRQRQKNRLSSVRKGIES |
| 13 | KANNQVTNFDNQTQKLNAKEINRRFELAKAYNRTLDPEYAHMLEITEMIGYIDIPSIKQKLPIYAGT |
| | TSSVLEKGSGHLEGTSLPIGGKSSHTVITAHRGLPKAKLFTDLDKLKKGKIFYIHNIKEVLAYKVDQ |
| | ISVVKPDNFSKLLVVKGKDYATLLTCTPYSINSHRLLVRGHRIKYVPPVKEKNYLMKELQTHYKLYF |
| | LLSILVILILVALLLYLKRKFKERKRKGNQK |
| 14 | HQSRAIMDYQDRVTHMDENDYKKIINRAKEYNKQFNSQLAIDKTGNMGYISIPKINIKLPLYHGTSE |
| | KVLQTSIGHLEGSSLPIGGDSTHSILSGHRGLPSSRLFSDLDKLKVGDHWTVSILNETYTYQVDQIR |
| | TVKPDDLRDLQIVKGKDYQTLVTCTPYGVNTHRLLVRGHRVPNDNGNALVVAEAIQIEPIYIAPFIA |
| | IFLTLILLLISLEVTRRARQRKKILKQAMRKEENNDL |
| 15 | ESNQQIADFDKEKATLDEADIDERMKLAQAFNDSLEYARMLEIHERMGHVEIPVIDVDLPVYAGTAE |
| | EVLQQGAGHLEGTSLPIGGNSTHAVITAHTGLPTAKMFTDLTKLKVGDKFYVHNIKEVMAYQVDQVK |
| | VIEPTNFDDLLIVPGHDYVTLLTCTPYMINTHRLLVRGHRIPYVAEVEEEFIAANKLSHLYRYLFYV |
| | AVGLIVILLWIIRRLRKKKKQPEKALKALKAARKEVKVEDGQQ |
| 16 | ESNEVIKEFDETVSQMDKAELEERWRLAQAFNATLKEYANMLKVHERIGYVEIPAIDQEIPMYVGTS |
| | EDILQKGAGLLEGASLPVGGENTHTVITAHRGLPTAELFSQLDKMKKGDIFYLHVLDQVLAYQVDQI |
| | VTVEPNDFEPVLIQHGEDYATLLTCTPYMINSHRLLVRGKRIPYTAPIAERNRAVRERGQFWLWLLL |
| | GAMAVILLLLYRVYRNRRIVKGLEKQLEGRHVKD |
| 17 | QSLGQVKGHATFVKSMTTEMYQEQQNHSLAYNQRLASQEVNYQVSDDPDAVYGYLSIPSLEIMEPVY |
| | LGADYHHLGMGLAHVDGTPLPLDGTGIRSVIAGHRAEPSHVFFRHLDQLKVGDALYYDNGQEIVEYQ |
| | MMDTEIILPSEWEKLESVSSKNIMTLITCDPIPTFNKRLLVNFERVAVYQKSDPQTAAVARVAFTKE |
| | GQSVSRVATSQWLYRGLVVLAFLGILFVLWKLARLLRGK |
| 18 | RDRQLLSTYHKQVTQKKPSEMEEVWQKAKAYNARLNYESLLQIENNDIMGYVEVPSIKVTLPIYHYT |
| | TDEVLTKGAGHLFGSALPVGGDGTHTVISAHRGLPSAEMFTNLNLVKKGDTFYFRVLNKVLAYKVDQ |
| | ILTVEPDQVTSLSGVMGKDYATLVTCTPYGVNTKRLLVRGHRIAYHYKKYQQAKKAMKLVDKSRMWA |
| | EVVCAAFGVVIAIILVFMYSRVSAKKSK |
| 19 | EYARMLEVKEQIGHVIIPRINQDIPIYAGSAEENLQRGVGHLEGTSLPVGGESTHAVLTAHRGLPTA |
| | KLFTNLDKVTVGDRFYIEHIGGKIAYQVDQIKVIAPDQLEDLYVIQGEDHVTLLTCTPYMINSHRLL |
| | VRGKRIPYVEKTVQKDSKTFRQQQYLTYAMWVVVGLILLSLLIWFKKTKQKKRRKNEKAASQNSHNN |
| | SK |
| 20 | EYARMLEVHEQIGHVAIPSIGVDIPIYAGTSETVLQKGSGHLEGTSLPVGGLSTHSVLTAHRGLPTA |
| | RLFTDLNKVKKGQIFYVTNIKETLAYKVVSIKVVDPTALSEVKIVNGKDYITLLTCTPYMINSHRLL |
| | VKGERIPYDSTEAEKHKEQTVQDYRLSLVLKILLVLLIGLFIVIMMRRWMQHRQ |
| 21 | EYARMLEVSEKIGIISVPKIGQKLPIFAGSSQEVLSKGAGHLEGTSLPIGGNSTHTVITAHSGIPDK |
| | ELFSNLKKLKKGDKFYIQNIKETIAYQVDQIKVVTPDNFSDLLVVPGHDYATLLTCTPIMVNTHRLL |
| | VRGHRIPYKGPIDEKLIKDGHLNTIYRYLFYISLVIIAWLLWLIKRQRQKNRLSSVRKGIES |
| 22 | EYAHMLEITEMIGYIDIPSIKQKLPIYAGTTSSVLEKGSGHLEGTSLPIGGKSSHTVITAHRGLPKA |
| | KLFTDLDKLKKGKIFYIHNIKEVLAYKVDQISVVKPDNFSKLLVVKGKDYATLLTCTPYSINSHRLL |
| | VRGHRIKYVPPVKEKNYLMKELQTHYKLYFLLSILVILILVALLLYLKRKFKERKRKGNQK |
| 23 | NSQLAIDKTGNMGYISIPKINIKLPLYHGTSEKVLQTSIGHLEGSSLPIGGDSTHSILSGHRGLPSS |
| | RLFSDLDKLKVGDHWTVSILNETYTYQVDQIRTVKPDDLRDLQIVKGKDYQTLVTCTPYGVNTHRLL |
| | VRGHRVPNDNGNALVVAEAIQIEPIYIAPFIAIFLTLILLLISLEVTRRARQRKKILKQAMRKEENN |
| | DL |
| 24 | EYARMLEIHERMGHVEIPVIDVDLPVYAGTAEEVLQQGAGHLEGTSLPIGGNSTHAVITAHTGLPTA |
| | KMFTDLTKLKVGDKFYVHNIKEVMAYQVDQVKVIEPTNFDDLLIVPGHDYVTLLTCTPYMINTHRLL |
| | VRGHRIPYVAEVEEEFIAANKLSHLYRYLFYVAVGLIVILLWIIRRLRKKKKQPEKALKALKAARKE |
| | VKVEDGQQ |
| 25 | EYANMLKVHERIGYVEIPAIDQEIPMYVGTSEDILQKGAGLLEGASLPVGGENTHTVITAHRGLPTA |
| | ELFSQLDKMKKGDIFYLHVLDQVLAYQVDQIVTVEPNDFEPVLIQHGEDYATLLTCTPYMINSHRLL |
| | VRGKRIPYTAPIAERNRAVRERGQFWLWLLLGAMAVILLLLYRVYRNRRIVKGLEKQLEGRHVKD |
| 26 | EVNYQVSDDPDAVYGYLSIPSLEIMEPVYLGADYHHLGMGLAHVDGTPLPLDGTGIRSVIAGHRAEP |
| | SHVFFRHLDQLKVGDALYYDNGQEIVEYQMMDTEIILPSEWEKLESVSSKNIMTLITCDPIPTFNKR |
| | LLVNFERVAVYQKSDPQTAAVARVAFTKEGQSVSRVATSQWLYRGLVVLAFLGILFVLWKLARLLRG |
| | K |
| 27 | HDKNYESLLQIENNDIMGYVEVPSIKVTLPIYHYTTDEVLTKGAGHLFGSALPVGGDGTHTVISAHR |
| | GLPSAEMFTNLNLVKKGDTFYFRVLNKVLAYKVDQILTVEPDQVTSLSGVMGKDYATLVTCTPYGVN |
| | TKRLLVRGHRIAYHYKKYQQAKKAMKLVDKSRMWAEVVCAAFGVVIAIILVFMYSRVSAKKSK |
| 28 | IVSQVMYFQASHANINAFKEAVTKIDRVEINRRLELAYAYNASIAGAKTNGEYPALKSAEQKQAGVV |
| | EYARMLEVKEQIGHVIIPRINQDIPIYAGSAEENLQRGVGHLEGTSLPVGGESTHAVLTAHRGLPTA |
| | KLFTNLDKVTVGDRFYIEHIGGKIAYQVDQIKVIAPDQLEDLYVIQGEDHVTLLTCTPYMINSHRLL |
| | VRGKRIPYVEKTVQKDSKTFRQQQYLTYAMWVVVGLILLSLLIWFKKTKQKKRRKNEKAASQNSHNN |
| | SK |
| 29 | ASHQNINQFKREVAKIDTNTVERRIALANAYNETLSRNPLLITSKQKEGLREYARMLEVHEQIGHVA |
| | IPSIGVDIPIYAGTSETVLQKGSGHLEGTSLPVGGLSTHSVLTAHRGLPTARLFTDLNKVKKGQIFY |
| | VTNIKETLAYKVVSIKVVDPTALSEVKIVNGKDYITLLTCTPYMINSHRLLVKGERIPYDSTEAEKH |
| | KEQTVQDYRLSLVLKILLVLLIGLFIVIMMRRWMQHRQ |
| 30 | ESNNQTQDFERAAKKLSQKEINRRMALAQAYNDSLNNVHLEEKKRIQKGIAEYARMLEVSEKIGIIS |
| | VPKIGQKLPIFAGSSQEVLSKGAGHLEGTSLPIGGNSTHTVITAHSGIPDKELFSNLKKLKKGDKFY |
| | IQNIKETIAYQVDQIKVVTPDNFSDLLVVPGHDYATLLTCTPIMVNTHRLLVRGHRIPYKGPIDEKL |
| | IKDGHLNTIYRYLFYISLVIIAWLLWLIKRQRQKNRLSSVRKGIES |
| 31 | KANNQVTNFDNQTQKLNAKEINRRFELAKAYNRTLDPSRLSTEKEKKGIAEYAHMLEITEMIGYIDI |
| | PSIKQKLPIYAGTTSSVLEKGSGHLEGTSLPIGGKSSHTVITAHRGLPKAKLFTDLDKLKKGKIFYI |
| | HNIKEVLAYKVDQISVVKPDNFSKLLVVKGKDYATLLTCTPYSINSHRLLVRGHRIKYVPPVKEKNY |
| | LMKELQTHYKLYFLLSILVILILVALLLYLKRKFKERKRKGNQK |
| 32 | HQSRAIMDYQDRVTHMDENDYKKIINRAKEYNKQFKTSGHMTSQERLDYNSQLAIDKTGNMGYISIP |
| | KINIKLPLYHGTSEKVLQTSIGHLEGSSLPIGGDSTHSILSGHRGLPSSRLFSDLDKLKVGDHWTVS |
| | ILNETYTYQVDQIRTVKPDDLRDLQIVKGKDYQTLVTCTPYGVNTHRLLVRGHRVPNDNGNALVVAE |
| | AIQIEPIYIAPFIAIFLTLILLLISLEVTRRARQRKKILKQAMRKEENNDL |
| 33 | ESNQQIADFDKEKATLDEADIDERMKLAQAFNDSLNNVVSGSEEMKKKGRAEYARMLEIHERMGHVE |
| | IPVIDVDLPVYAGTAEEVLQQGAGHLEGTSLPIGGNSTHAVITAHTGLPTAKMFTDLTKLKVGDKFY |
| | VHNIKEVMAYQVDQVKVIEPTNFDDLLIVPGHDYVTLLTCTPYMINTHRLLVRGHRIPYVAEVEEEF |
| | IAANKLSHLYRYLFYVAVGLIVILLWIIRRLRKKKKQPEKALKALKAARKEVKVEDGQQ |
| 34 | ESNEVIKEFDETVSQMDKAELEERWRLAQAFNATLKPSEILTEQEKKKGVSEYANMLKVHERIGYVE |
| | IPAIDQEIPMYVGTSEDILQKGAGLLEGASLPVGGENTHTVITAHRGLPTAELFSQLDKMKKGDIFY |
| | LHVLDQVLAYQVDQIVTVEPNDFEPVLIQHGEDYATLLTCTPYMINSHRLLVRGKRIPYTAPIAERN |
| | RAVRERGQFWLWLLLGAMAVILLLLYRVYRNRRIVKGLEKQLEGRHVKD |
| 35 | QSLGQVKGHATFVKSMTTEMYQEQQNHSLAYNQRLASQNRIVLAEGYEVNYQVSDDPDAVYGYLSIP |
| | SLEIMEPVYLGADYHHLGMGLAHVDGTPLPLDGTGIRSVIAGHRAEPSHVFFRHLDQLKVGDALYYD |
| | NGQEIVEYQMMDTEIILPSEWEKLESVSSKNIMTLITCDPIPTFNKRLLVNFERVAVYQKSDPQTAA |
| | VARVAFTKEGQSVSRVATSQWLYRGLVVLAFLGILFVLWKLARLLRGK |
| 36 | RDRQLLSTYHKQVTQKKPSEMEEVWQKAKAYNARLGIQPVPSFRDGIHDKNYESLLQIENNDIMGYV |
| | EVPSIKVTLPIYHYTTDEVLTKGAGHLFGSALPVGGDGTHTVISAHRGLPSAEMFTNLNLVKKGDTF |
| | YFRVLNKVLAYKVDQILTVEPDQVTSLSGVMGKDYATLVTCTPYGVNTKRLLVRGHRIAYHYKKYQQ |
| | AKKAMKLVDKSRMWAEVVCAAFGVVIAIILVFMYSRVSAKKSK |
| 37 | IVSQVMYFQASHANINAFKEAVTKIDRVEINRRLELAYAYNASIAGAKTNGEYPALKAPYSAEQKQA |
| | GVVEYARMLEVKEQIGHVIIPRINQDIPIYAGSAEENLQRGVGHLEGTSLPVGGESTHAVLTAHRGL |
| | PTAKLFTNLDKVTVGDRFYIEHIGGKIAYQVDQIKVIAPDQLEDLYVIQGEDHVTLLTCTPYMINSH |
| | RLLVRGKRIPYVEKTVQKDSKTFRQQQYLTYAMWVVVGLILLSLLIWFKKTKQKKRRKNEKAASQNS |
| | HNNSK |
| 38 | ASHQNINQFKREVAKIDTNTVERRIALANAYNETLSRNPLLIAPFTSKQKEGLREYARMLEVHEQIG |
| | HVAIPSIGVDIPIYAGTSETVLQKGSGHLEGTSLPVGGLSTHSVLTAHRGLPTARLFTDLNKVKKGQ |
| | IFYVTNIKETLAYKVVSIKVVDPTALSEVKIVNGKDYITLLTCTPYMINSHRLLVKGERIPYDSTEA |
| | EKHKEQTVQDYRLSLVLKILLVLLIGLFIVIMMRRWMQHRQ |
| 39 | ESNNQTQDFERAAKKLSQKEINRRMALAQAYNDSLNNVHLEAPYEKKRIQKGIAEYARMLEVSEKIG |
| | IISVPKIGQKLPIFAGSSQEVLSKGAGHLEGTSLPIGGNSTHTVITAHSGIPDKELFSNLKKLKKGD |
| | KFYIQNIKETIAYQVDQIKVVTPDNFSDLLVVPGHDYATLLTCTPIMVNTHRLLVRGHRIPYKGPID |
| | EKLIKDGHLNTIYRYLFYISLVIIAWLLWLIKRQRQKNRLSSVRKGIES |
| 40 | KANNQVTNFDNQTQKLNAKEINRRFELAKAYNRTLDPSRLSAPYTEKEKKGIAEYAHMLEITEMIGY |
| | IDIPSIKQKLPIYAGTTSSVLEKGSGHLEGTSLPIGGKSSHTVITAHRGLPKAKLFTDLDKLKKGKI |
| | FYIHNIKEVLAYKVDQISVVKPDNFSKLLVVKGKDYATLLTCTPYSINSHRLLVRGHRIKYVPPVKE |
| | KNYLMKELQTHYKLYFLLSILVILILVALLLYLKRKFKERKRKGNQK |
| 41 | HQSRAIMDYQDRVTHMDENDYKKIINRAKEYNKQFKTSGAKWHMTSQERLDYNSQLAIDKTGNMGYI |
| | SIPKINIKLPLYHGTSEKVLQTSIGHLEGSSLPIGGDSTHSILSGHRGLPSSRLFSDLDKLKVGDHW |
| | TVSILNETYTYQVDQIRTVKPDDLRDLQIVKGKDYQTLVTCTPYGVNTHRLLVRGHRVPNDNGNALV |
| | VAEAIQIEPIYIAPFIAIFLTLILLLISLEVTRRARQRKKILKQAMRKEENNDL |
| 42 | ESNQQIADFDKEKATLDEADIDERMKLAQAFNDSLNNVVSGAPWSEEMKKKGRAEYARMLEIHERMG |
| | HVEIPVIDVDLPVYAGTAEEVLQQGAGHLEGTSLPIGGNSTHAVITAHTGLPTAKMFTDLTKLKVGD |
| | KFYVHNIKEVMAYQVDQVKVIEPTNFDDLLIVPGHDYVTLLTCTPYMINTHRLLVRGHRIPYVAEVE |
| | EEFIAANKLSHLYRYLFYVAVGLIVILLWIIRRLRKKKKQPEKALKALKAARKEVKVEDGQQ |
| 43 | ESNEVIKEFDETVSQMDKAELEERWRLAQAFNATLKPSEILAPFTEQEKKKGVSEYANMLKVHERIG |
| | YVEIPAIDQEIPMYVGTSEDILQKGAGLLEGASLPVGGENTHTVITAHRGLPTAELFSQLDKMKKGD |
| | IFYLHVLDQVLAYQVDQIVTVEPNDFEPVLIQHGEDYATLLTCTPYMINSHRLLVRGKRIPYTAPIA |
| | ERNRAVRERGQFWLWLLLGAMAVILLLLYRVYRNRRIVKGLEKQLEGRHVKD |
| 44 | QSLGQVKGHATFVKSMTTEMYQEQQNHSLAYNQRLASQNRIVAPFLAEGYEVNYQVSDDPDAVYGYL |
| | SIPSLEIMEPVYLGADYHHLGMGLAHVDGTPLPLDGTGIRSVIAGHRAEPSHVFFRHLDQLKVGDAL |
| | YYDNGQEIVEYQMMDTEIILPSEWEKLESVSSKNIMTLITCDPIPTFNKRLLVNFERVAVYQKSDPQ |
| | TAAVARVAFTKEGQSVSRVATSQWLYRGLVVLAFLGILFVLWKLARLLRGK |
| 45 | RDRQLLSTYHKQVTQKKPSEMEEVWQKAKAYNARLGIQPVPAAFSFRDGIHDKNYESLLQIENNDIM |
| | GYVEVPSIKVTLPIYHYTTDEVLTKGAGHLFGSALPVGGDGTHTVISAHRGLPSAEMFTNLNLVKKG |
| | DTFYFRVLNKVLAYKVDQILTVEPDQVTSLSGVMGKDYATLVTCTPYGVNTKRLLVRGHRIAYHYKK |
| | YQQAKKAMKLVDKSRMWAEVVCAAFGVVIAIILVFMYSRVSAKKSK |
| 46 | IVSQVMYFQASHANINAFKEAVTKIDRVEINRRLELAYAYNASIAGAKTNGEYPALKDPASAEQKQA |
| | GVVEYARMLEVKEQIGHVIIPRINQDIPIYAGSAEENLQRGVGHLEGTSLPVGGESTHAVLTAHRGL |
| | PTAKLFTNLDKVTVGDRFYIEHIGGKIAYQVDQIKVIAPDQLEDLYVIQGEDHVTLLTCTPYMINSH |
| | RLLVRGKRIPYVEKTVQKDSKTFRQQQYLTYAMWVVVGLILLSLLIWFKKTKQKKRRKNEKAASQNS |
| | HNNSK |
| 47 | ASHQNINQFKREVAKIDTNTVERRIALANAYNETLSRNPLLIDPATSKQKEGLREYARMLEVHEQIG |
| | HVAIPSIGVDIPIYAGTSETVLQKGSGHLEGTSLPVGGLSTHSVLTAHRGLPTARLFTDLNKVKKGQ |
| | IFYVTNIKETLAYKVVSIKVVDPTALSEVKIVNGKDYITLLTCTPYMINSHRLLVKGERIPYDSTEA |
| | EKHKEQTVQDYRLSLVLKILLVLLIGLFIVIMMRRWMQHRQ |
| 48 | ESNNQTQDFERAAKKLSQKEINRRMALAQAYNDSLNNVHLEDPAEKKRIQKGIAEYARMLEVSEKIG |
| | IISVPKIGQKLPIFAGSSQEVLSKGAGHLEGTSLPIGGNSTHTVITAHSGIPDKELFSNLKKLKKGD |
| | KFYIQNIKETIAYQVDQIKVVTPDNFSDLLVVPGHDYATLLTCTPIMVNTHRLLVRGHRIPYKGPID |
| | EKLIKDGHLNTIYRYLFYISLVIIAWLLWLIKRQRQKNRLSSVRKGIES |
| 49 | KANNQVTNFDNQTQKLNAKEINRRFELAKAYNRTLDPSRLSDPATEKEKKGIAEYAHMLEITEMIGY |
| | IDIPSIKQKLPIYAGTTSSVLEKGSGHLEGTSLPIGGKSSHTVITAHRGLPKAKLFTDLDKLKKGKI |
| | FYIHNIKEVLAYKVDQISVVKPDNFSKLLVVKGKDYATLLTCTPYSINSHRLLVRGHRIKYVPPVKE |
| | KNYLMKELQTHYKLYFLLSILVILILVALLLYLKRKFKERKRKGNQK |
| 50 | HQSRAIMDYQDRVTHMDENDYKKIINRAKEYNKQFKTSGMKAHMTSQERLDYNSQLAIDKTGNMGYI |
| | SIPKINIKLPLYHGTSEKVLQTSIGHLEGSSLPIGGDSTHSILSGHRGLPSSRLFSDLDKLKVGDHW |
| | TVSILNETYTYQVDQIRTVKPDDLRDLQIVKGKDYQTLVTCTPYGVNTHRLLVRGHRVPNDNGNALV |
| | VAEAIQIEPIYIAPFIAIFLTLILLLISLEVTRRARQRKKILKQAMRKEENNDL |
| 51 | ESNQQIADFDKEKATLDEADIDERMKLAQAFNDSLNNVVSGDPASEEMKKKGRAEYARMLEIHERMG |
| | HVEIPVIDVDLPVYAGTAEEVLQQGAGHLEGTSLPIGGNSTHAVITAHTGLPTAKMFTDLTKLKVGD |
| | KFYVHNIKEVMAYQVDQVKVIEPTNFDDLLIVPGHDYVTLLTCTPYMINTHRLLVRGHRIPYVAEVE |
| | EEFIAANKLSHLYRYLFYVAVGLIVILLWIIRRLRKKKKQPEKALKALKAARKEVKVEDGQQ |
| 52 | ESNEVIKEFDETVSQMDKAELEERWRLAQAFNATLKPSEILDPATEQEKKKGVSEYANMLKVHERIG |
| | YVEIPAIDQEIPMYVGTSEDILQKGAGLLEGASLPVGGENTHTVITAHRGLPTAELFSQLDKMKKGD |
| | IFYLHVLDQVLAYQVDQIVTVEPNDFEPVLIQHGEDYATLLTCTPYMINSHRLLVRGKRIPYTAPIA |
| | ERNRAVRERGQFWLWLLLGAMAVILLLLYRVYRNRRIVKGLEKQLEGRHVKD |
| 53 | QSLGQVKGHATFVKSMTTEMYQEQQNHSLAYNQRLASQNRIVDPALAEGYEVNYQVSDDPDAVYGYL |
| | SIPSLEIMEPVYLGADYHHLGMGLAHVDGTPLPLDGTGIRSVIAGHRAEPSHVFFRHLDQLKVGDAL |
| | YYDNGQEIVEYQMMDTEIILPSEWEKLESVSSKNIMTLITCDPIPTFNKRLLVNFERVAVYQKSDPQ |
| | TAAVARVAFTKEGQSVSRVATSQWLYRGLVVLAFLGILFVLWKLARLLRGK |
| 54 | RDRQLLSTYHKQVTQKKPSEMEEVWQKAKAYNARLGIQPVPDAASFRDGIHDKNYESLLQIENNDIM |
| | GYVEVPSIKVTLPIYHYTTDEVLTKGAGHLFGSALPVGGDGTHTVISAHRGLPSAEMFTNLNLVKKG |
| | DTFYFRVLNKVLAYKVDQILTVEPDQVTSLSGVMGKDYATLVTCTPYGVNTKRLLVRGHRIAYHYKK |
| | YQQAKKAMKLVDKSRMWAEVVCAAFGVVIAIILVFMYSRVSAKKSK |
| 55 | IVSQVMYFQASHANINAFKEAVTKIDRVEINRRLELAYAYNASIAGAKTNGEYPALKAPASAEQKQA |
| | GVVEYARMLEVKEQIGHVIIPRINQDIPIYAGSAEENLQRGVGHLEGTSLPVGGESTHAVLTAHRGL |
| | PTAKLFTNLDKVTVGDRFYIEHIGGKIAYQVDQIKVIAPDQLEDLYVIQGEDHVTLLTCTPYMINSH |
| | RLLVRGKRIPYVEKTVQKDSKTFRQQQYLTYAMWVVVGLILLSLLIWFKKTKQKKRRKNEKAASQNS |
| | HNNSK |
| 56 | ASHQNINQFKREVAKIDTNTVERRIALANAYNETLSRNPLLIAPATSKQKEGLREYARMLEVHEQIG |
| | HVAIPSIGVDIPIYAGTSETVLQKGSGHLEGTSLPVGGLSTHSVLTAHRGLPTARLFTDLNKVKKGQ |
| | IFYVTNIKETLAYKVVSIKVVDPTALSEVKIVNGKDYITLLTCTPYMINSHRLLVKGERIPYDSTEA |
| | EKHKEQTVQDYRLSLVLKILLVLLIGLFIVIMMRRWMQHRQ |
| 57 | ESNNQTQDFERAAKKLSQKEINRRMALAQAYNDSLNNVHLEAPAEKKRIQKGIAEYARMLEVSEKIG |
| | IISVPKIGQKLPIFAGSSQEVLSKGAGHLEGTSLPIGGNSTHTVITAHSGIPDKELFSNLKKLKKGD |
| | KFYIQNIKETIAYQVDQIKVVTPDNFSDLLVVPGHDYATLLTCTPIMVNTHRLLVRGHRIPYKGPID |
| | EKLIKDGHLNTIYRYLFYISLVIIAWLLWLIKRQRQKNRLSSVRKGIES |
| 58 | KANNQVTNFDNQTQKLNAKEINRRFELAKAYNRTLDPSRLSAPATEKEKKGIAEYAHMLEITEMIGY |
| | IDIPSIKQKLPIYAGTTSSVLEKGSGHLEGTSLPIGGKSSHTVITAHRGLPKAKLFTDLDKLKKGKI |
| | FYIHNIKEVLAYKVDQISVVKPDNFSKLLVVKGKDYATLLTCTPYSINSHRLLVRGHRIKYVPPVKE |
| | KNYLMKELQTHYKLYFLLSILVILILVALLLYLKRKFKERKRKGNQK |
| 59 | HQSRAIMDYQDRVTHMDENDYKKIINRAKEYNKQFKTSGAKAHMTSQERLDYNSQLAIDKTGNMGYI |
| | SIPKINIKLPLYHGTSEKVLQTSIGHLEGSSLPIGGDSTHSILSGHRGLPSSRLFSDLDKLKVGDHW |
| | TVSILNETYTYQVDQIRTVKPDDLRDLQIVKGKDYQTLVTCTPYGVNTHRLLVRGHRVPNDNGNALV |
| | VAEAIQIEPIYIAPFIAIFLTLILLLISLEVTRRARQRKKILKQAMRKEENNDL |
| 60 | ESNQQIADFDKEKATLDEADIDERMKLAQAFNDSLNNVVSGAPASEEMKKKGRAEYARMLEIHERMG |
| | HVEIPVIDVDLPVYAGTAEEVLQQGAGHLEGTSLPIGGNSTHAVITAHTGLPTAKMFTDLTKLKVGD |
| | KFYVHNIKEVMAYQVDQVKVIEPTNFDDLLIVPGHDYVTLLTCTPYMINTHRLLVRGHRIPYVAEVE |
| | EEFIAANKLSHLYRYLFYVAVGLIVILLWIIRRLRKKKKQPEKALKALKAARKEVKVEDGQQ |
| 61 | ESNEVIKEFDETVSQMDKAELEERWRLAQAFNATLKPSEILAPATEQEKKKGVSEYANMLKVHERIG |
| | YVEIPAIDQEIPMYVGTSEDILQKGAGLLEGASLPVGGENTHTVITAHRGLPTAELFSQLDKMKKGD |
| | IFYLHVLDQVLAYQVDQIVTVEPNDFEPVLIQHGEDYATLLTCTPYMINSHRLLVRGKRIPYTAPIA |
| | ERNRAVRERGQFWLWLLLGAMAVILLLLYRVYRNRRIVKGLEKQLEGRHVKD |
| 62 | QSLGQVKGHATFVKSMTTEMYQEQQNHSLAYNQRLASQNRIVAPALAEGYEVNYQVSDDPDAVYGYL |
| | SIPSLEIMEPVYLGADYHHLGMGLAHVDGTPLPLDGTGIRSVIAGHRAEPSHVFFRHLDQLKVGDAL |
| | YYDNGQEIVEYQMMDTEIILPSEWEKLESVSSKNIMTLITCDPIPTFNKRLLVNFERVAVYQKSDPQ |
| | TAAVARVAFTKEGQSVSRVATSQWLYRGLVVLAFLGILFVLWKLARLLRGK |
| 63 | RDRQLLSTYHKQVTQKKPSEMEEVWQKAKAYNARLGIQPVPAAASFRDGIHDKNYESLLQIENNDIM |
| | GYVEVPSIKVTLPIYHYTTDEVLTKGAGHLFGSALPVGGDGTHTVISAHRGLPSAEMFTNLNLVKKG |
| | DTFYFRVLNKVLAYKVDQILTVEPDQVTSLSGVMGKDYATLVTCTPYGVNTKRLLVRGHRIAYHYKK |
| | YQQAKKAMKLVDKSRMWAEVVCAAFGVVIAIILVFMYSRVSAKKSK |
| 64 | IVSQVMYFQASHANINAFKEAVTKIDRVEINRRLELAYAYNASIAGAKTNGEYPALKAAASAEQKQA |
| | GVVEYARMLEVKEQIGHVIIPRINQDIPIYAGSAEENLQRGVGHLEGTSLPVGGESTHAVLTAHRGL |
| | PTAKLFTNLDKVTVGDRFYIEHIGGKIAYQVDQIKVIAPDQLEDLYVIQGEDHVTLLTCTPYMINSH |
| | RLLVRGKRIPYVEKTVQKDSKTFRQQQYLTYAMWVVVGLILLSLLIWFKKTKQKKRRKNEKAASQNS |
| | HNNSK |
| 65 | ASHQNINQFKREVAKIDTNTVERRIALANAYNETLSRNPLLIAAATSKQKEGLREYARMLEVHEQIG |
| | HVAIPSIGVDIPIYAGTSETVLQKGSGHLEGTSLPVGGLSTHSVLTAHRGLPTARLFTDLNKVKKGQ |
| | IFYVTNIKETLAYKVVSIKVVDPTALSEVKIVNGKDYITLLTCTPYMINSHRLLVKGERIPYDSTEA |
| | EKHKEQTVQDYRLSLVLKILLVLLIGLFIVIMMRRWMQHRQ |
| 66 | ESNNQTQDFERAAKKLSQKEINRRMALAQAYNDSLNNVHLEAAAEKKRIQKGIAEYARMLEVSEKIG |
| | IISVPKIGQKLPIFAGSSQEVLSKGAGHLEGTSLPIGGNSTHTVITAHSGIPDKELFSNLKKLKKGD |
| | KFYIQNIKETIAYQVDQIKVVTPDNFSDLLVVPGHDYATLLTCTPIMVNTHRLLVRGHRIPYKGPID |
| | EKLIKDGHLNTIYRYLFYISLVIIAWLLWLIKRQRQKNRLSSVRKGIES |
| 67 | KANNQVTNFDNQTQKLNAKEINRRFELAKAYNRTLDPSRLSAAATEKEKKGIAEYAHMLEITEMIGY |
| | IDIPSIKQKLPIYAGTTSSVLEKGSGHLEGTSLPIGGKSSHTVITAHRGLPKAKLFTDLDKLKKGKI |
| | FYIHNIKEVLAYKVDQISVVKPDNFSKLLVVKGKDYATLLTCTPYSINSHRLLVRGHRIKYVPPVKE |
| | KNYLMKELQTHYKLYFLLSILVILILVALLLYLKRKFKERKRKGNQK |
| 68 | HQSRAIMDYQDRVTHMDENDYKKIINRAKEYNKQFKTSGAAAHMTSQERLDYNSQLAIDKTGNMGYI |
| | SIPKINIKLPLYHGTSEKVLQTSIGHLEGSSLPIGGDSTHSILSGHRGLPSSRLFSDLDKLKVGDHW |
| | TVSILNETYTYQVDQIRTVKPDDLRDLQIVKGKDYQTLVTCTPYGVNTHRLLVRGHRVPNDNGNALV |
| | VAEAIQIEPIYIAPFIAIFLTLILLLISLEVTRRARQRKKILKQAMRKEENNDL |
| 69 | ESNQQIADFDKEKATLDEADIDERMKLAQAFNDSLNNVVSGAAASEEMKKKGRAEYARMLEIHERMG |
| | HVEIPVIDVDLPVYAGTAEEVLQQGAGHLEGTSLPIGGNSTHAVITAHTGLPTAKMFTDLTKLKVGD |
| | KFYVHNIKEVMAYQVDQVKVIEPTNFDDLLIVPGHDYVTLLTCTPYMINTHRLLVRGHRIPYVAEVE |
| | EEFIAANKLSHLYRYLFYVAVGLIVILLWIIRRLRKKKKQPEKALKALKAARKEVKVEDGQQ |
| 70 | ESNEVIKEFDETVSQMDKAELEERWRLAQAFNATLKPSEILAAATEQEKKKGVSEYANMLKVHERIG |
| | YVEIPAIDQEIPMYVGTSEDILQKGAGLLEGASLPVGGENTHTVITAHRGLPTAELFSQLDKMKKGD |
| | IFYLHVLDQVLAYQVDQIVTVEPNDFEPVLIQHGEDYATLLTCTPYMINSHRLLVRGKRIPYTAPIA |
| | ERNRAVRERGQFWLWLLLGAMAVILLLLYRVYRNRRIVKGLEKQLEGRHVKD |
| 71 | QSLGQVKGHATFVKSMTTEMYQEQQNHSLAYNQRLASQNRIVAAALAEGYEVNYQVSDDPDAVYGYL |
| | SIPSLEIMEPVYLGADYHHLGMGLAHVDGTPLPLDGTGIRSVIAGHRAEPSHVFFRHLDQLKVGDAL |
| | YYDNGQEIVEYQMMDTEIILPSEWEKLESVSSKNIMTLITCDPIPTFNKRLLVNFERVAVYQKSDPQ |
| | TAAVARVAFTKEGQSVSRVATSQWLYRGLVVLAFLGILFVLWKLARLLRGK |
| 72 | MKLSKKLLFSAAVLTMVAGSTVEPVAQFATGMSIVRAAEVSQERPAKTTVNIYKLQADSYKSEITSN |
| | GGIENKDGEVISNYAKLGDNVKGLQGVQFKRYKVKTDISVDELKKLTTVEAADAKVGTILEEGVSLP |
| | QKTNAQGLVVDALDSKSNVRYLYVEDLKNSPSNITKAYAVPFVLELPVANSTGTGFLSEINTYPKNV |
| | VTDEPKTDKDVKKLGQDDAGYTIGEEFKWFLKSTIPANLGDYEKFEITDKFADGLTYKSVGKIKIGS |
| | KTLNRDEHYTIDEPTVDNQNTLKITFKPEKFKEIAELLKGMTLVKNQDALDKATANTDDAAFLEIPV |
| | ASTINEKAVLGKAIENTFELQYDHTPDKADNPKPSNPPRKPEVHTGGKRFVKKDSTETQTLGGAEFD |
| | LLASDGTAVKWTDALIKANTNKNYIAGEAVTGQPIKLKSHTDGTFEIKGLAYAVDANAEGTAVTYKL |
| | KETKAPEGYVIPDKEIEFTVSQTSYNTKPTDITVDSADATPDTIKNNKRPSIPNTGGIGTAIFVAIG |
| | AAVMAFAVKGMKRRTKDN |
| 73 | AEVSQERPAKTTVNIYKLQADSYKSEITSNGGIENKDGEVISNYAKLGDNVKGLQGVQFKRYKVKTD |
| | ISVDELKKLTTVEAADAKVGTILEEGVSLPQKTNAQGLVVDALDSKSNVRYLYVEDLKNSPSNITKA |
| | YAVPFVLELPVANSTGTGFLSEINTYPKNVVTDEPKTDKDVKKLGQDDAGYTIGEEFKWFLKSTIPA |
| | NLGDYEKFEITDKFADGLTYKSVGKIKIGSKTLNRDEHYTIDEPTVDNQNTLKITFKPEKFKEIAEL |
| | LKGMTLVKNQDALDKATANTDDAAFLEIPVASTINEKAVLGKAIENTFELQYDHTPDKADNPKPSNP |
| | PRKPEVHTGGKRFVKKDSTETQTLGGAEFDLLASDGTAVKWTDALIKANTNKNYIAGEAVTGQPIKL |
| | KSHTDGTFEIKGLAYAVDANAEGTAVTYKLKETKAPEGYVIPDKEIEFTVSQTSYNTKPTDITVDSA |
| | DATPDTIKNNKRPSIPNTGGIGTAIFVAIGAAVMAFAVKGMKRRTKDN |
| 74 | MKRINKYFAMFSALLLTLTSLLSVAPAFADEATTNTVTLHKILQTESNLNKSNFPGTTGLNGKDYKG |
| | GAISDLAGYFGEGSKEIEGAFFALALKEDKSGKVQYVKAKEGNKLTPALINKDGTPEITVNIDEAVS |
| | GLTPEGDTGLVFNTKGLKGEFKIVEVKSKSTYNNNGSLLAASKAVPVNITLPLVNEDGVVADAHVYP |
| | KNTEEKPEIDKNFAKTNDLTALTDVNRLLTAGANYGNYARDKATATAEIGKVVPYEVKTKIHKGSKY |
| | ENLVWTDIMSNGLTMGSTVSLKASGTTETFAKDTDYELSIDARGFTLKFTADGLGKLEKAAKTADIE |
| | FTLTYSATVNGQAIIDNPESNDIKLSYGNKPGKDLTELPVTPSKGEVTVAKTWSDGIAPDGVNVVYT |
| | LKDKDKTVASVSLTKTSKGTIDLGNGIKFEVSGNFSGKFTGLENKSYMISERVSGYGSAINLENGKV |
| | TITNTKDSDNPTPLNPTEPKVETHGKKFVKTNEQGDRLAGAQFVVKNSAGKYLALKADQSEGQKTLA |
| | AKKIALDEATAAYNKLSATDQKGEKGITAKELIKTKQADYDAAFIEARTAYEWITDKARAITYTSND |
| | QGQFEVTGLADGTYNLEETLAPAGFAKLAGNIKFVVNQGSYITGGNIDYVANSNQKDATRVENKKVT |
| | IPQTGGIGTILFTIIGLSIMLGAVVIMKRRQSKEA |
| 75 | MKKINKYFAVFSALLLTVTSLFSVAPVFAEEAKTTDTVTLHKIVMPRTAFDGFTAGTKGKDNTDYVG |
| | KQIEDLKTYFGSGEAKEIAGAYFAFKNEAGTKYITENGEEVDTLDTTDAKGCAVLKGLTTDNGFKFN |
| | TSKLTGTYQIVELKEKSTYNNDGSILADSKAVPVKITLPLVNDNGVVKDAHVYPKNTETKPQVDKNF |
| | ADKELDYANNKKDKGTVSASVGDVKKYHVGTKILKGSDYKKLIWTDSMTKGLTFNNDIAVTLDGATL |
| | DATNYKLVADDQGFRLVLTDKGLEAVAKAAKTKDVEIKITYSATLNGSAVVEVLETNDVKLDYGNNP |
| | TIENEPKEGIPVDKKITVNKTWAVDGNEVNKADETVDAVFTLQVKDGDKWVNVDSAKATAATSFKHT |
| | FENLDNAKTYRVIERVSGYAPEYVSFVNGVVTIKNNKDSNEPTPINPSEPKVVTYGRKFVKTNKDGK |
| | ERLAGATFLVKKDGKYLARKSGVATDAEKAAVDSTKSALDAAVKAYNDLTKEKQEGQDGKSALATVS |
| | EKQKAYNDAFVKANYSYEWVEDKNAKNVVKLISNDKGQFEITGLTEGQYSLEETQAPTGYAKLSGDV |
| | SFNVNATSYSKGSAQDIEYTQGSKTKDAQQVINKKVTIPQTGGIGTIFFTIIGLSIMLGAVVIMKRR |
| | QSEEV |
| 76 | MKKINKCLTMFSTLLLILTSLFSVAPAFADDATTDTVTLHKIVMPQAAFDNFTEGTKGKNDSDYVGK |
| | QINDLKSYFGSTDAKEIKGAFFVFKNETGTKFITENGKEVDTLEAKDAEGGAVLSGLTKDNGFVFNT |
| | AKLKGIYQIVELKEKSNYDNNGSILADSKAVPVKITLPLVNNQGVVKDAHIYPKNTETKPQVDKNFA |
| | DKDLDYTDNRKDKGVVSATVGDKKEYIVGTKILKGSDYKKLVWTDSMTKGLTFNNNVKVTLDGEDFP |
| | VLNYKLVTDDQGFRLALNATGLAAVAAAAKDKDVEIKITYSATVNGSTTVEIPETNDVKLDYGNNPT |
| | EESEPQEGTPANQEIKVIKDWAVDGTITDANVAVKAIFTLQEKQTDGTWVNVASHEATKPSRFEHTF |
| | TGLDNAKTYRVVERVSGYTPEYVSFKNGVVTIKNNKNSNDPTPINPSEPKVVTYGRKFVKTNQANTE |
| | RLAGATFLVKKEGKYLARKAGAATAEAKAAVKTAKLALDEAVKAYNDLTKEKQEGQEGKTALATVDQ |
| | KQKAYNDAFVKANYSYEWVADKKADNVVKLISNAGGQFEITGLDKGTYGLEETQAPAGYATLSGDVN |
| | FEVTATSYSKGATTDIAYDKGSVKKDAQQVQNKKVTIPQTGGIGTILFTIIGLSIMLGAVVIMKKRQ |
| | SEEA |
| 77 | MKRINKYFAMFSALLLILTSLLSVAPVFAAEMGNITKTVTLHKIVQTSDNLAKPNFPGINGLNGTKY |
| | MGQKLTDISGYFGQGSKEIAGAFFAVMNESQTKYITESGTEVESIDAAGVLKGLTTENGITFNTANL |
| | KGTYQIVELLDKSNYKNGDKVLADSKAVPVKITLPLYNEEGIVVDAEVYPKNTEEAPQIDKNFAKAN |
| | KLLNDSDNSAIAGGADYDKYQAEKAKATAEIGQEIPYEVKTKIQKGSKYKNLAWVDTMSNGLTMGNT |
| | VNLEASSGSFVEGTDYNVERDDRGFTLKFTDTGLTKLQKEAETQAVEFTLTYSATVNGAAIDDKPES |
| | NDIKLQYGNKPGKKVKEIPVTPSNGEITVSKTWDKGSDLENANVVYTLKDGGTAVASVSLTKTTPNG |
| | EINLGNGIKFTVTGAFAGKFSGLTDSKTYMISERIAGYGNTITTGAGSAAITNTPDSDNPTPLNPTE |
| | PKVVTHGKKFVKTSSTETERLQGAQFVVKDSAGKYLALKSSATISAQTTAYTNAKTALDAKIAAYNK |
| | LSADDQKGTKGETAKAEIKTAQDAYNAAFIVARTAYEWVTNKEDANVVKVTSNADGQFEVSGLATGD |
| | YKLEETQAPAGYAKLAGDVDFKVGNSSKADDSGNIDYTASSNKKDAQRIENKKVTIPQTGGIGTILF |
| | TIIGLSIMLGAVIIMKRRQSEEA |
| 78 | MKKINKYFAVFSALLLTVTSLLSVAPAFADEATTNTVTLHKILQTESNLNKSNFPGTTGLNGDDYKG |
| | ESISDLAEYFGSGSKEIDGAFFALALEEEKDGVVQYVKAKANDKLTPDLITKGTPATTTKVEEAVGG |
| | LTTGTGIVFNTAGLKGNFKIIELKDKSTYNNNGSLLAASKAVPVKITLPLVSKDGVVKDAHVYPKNT |
| | ETKPEVDKNFAKTNDLTALKDATLLKAGADYKNYSATKATVTAEIGKVIPYEVKTKVLKGSKYEKLV |
| | WTDTMSNGLTMGDDVNLAVSGTTTTFIKDIDYTLSIDDRGFTLKFKATGLDKLEEAAKASDVEFTLT |
| | YKATVNGQAIIDNPEVNDIKLDYGNKPGTDLSEQPVTPEDGEVKVTKTWAAGANKADAKVVYTLKNA |
| | TKQVVASVALTAADTKGTINLGKGMTFEITGAFSGTFKGLQNKAYTVSERVAGYTNAINVTGNAVAI |
| | TNTPDSDNPTPLNPTQPKVETHGKKFVKVGDADARLAGAQFVVKNSAGKFLALKEDAAVSGAQTELA |
| | TAKTDLDNAIKAYNGLTKAQQEGADGTSAKELINTKQSAYDAAFIKARTAYTWVDEKTKAITFTSNN |
| | QGQFEVTGLEVGSYKLEETLAPAGYAKLSGDIEFTVGHDSYTSGDIKYKTDDASNNAQKVFNKKVTI |
| | PQTGGIGTILFTIIGLSIMLGAVVIMKRRQSEEA |
| 79 | MKKINKFFVAFSALLLILTSLLSVAPAFAEEERTTETVTLHKILQTETNLKNSAFPGTKGLDGTEYD |
| | GKAIDKLDSYFGNDSKDIGGAYFILANSKGEYIKANDKNKLKPEFSGNTPKTTLNISEAVGGLTEEN |
| | AGIKFETTGLRGDFQIIELKDKSTYNNGGAILADSKAVPVKITLPLINKDGVVKDAHVYPKNTETKP |
| | QIDKNFADKNLDYINNQKDKGTISATVGDVKKYTVGTKILKGSDYKKLVWTDSMTKGLTFNNDVTVT |
| | LDGANFEQSNYTLVADDQGFRLVLNATGLSKVAEAAKTKDVEIKINYSATVNGSTVVEKSENNDVKL |
| | DYGNNPTTENEPQTGNPVNKEITVRKTWAVDGNEVNKGDEKVDAVFTLQVKDSDKWVNVDSATATAA |
| | TDFKYTFKNLDNAKTYRVVERVSGYAPAYVSFVGGVVTIKNNKNSNDPTPINPSEPKVVTYGRKFVK |
| | TNQDGSERLAGATFLVKNSQSQYLARKSGVATNEAHKAVTDAKVQLDEAVKAYNKLTKEQQESQDGK |
| | AALNLIDEKQTAYNEAFAKANYSYEWVVDKNAANVVKLISNTAGKFEITGLNAGEYSLEETQAPTGY |
| | AKLSSDVSFKVNDTSYSEGASNDIAYDKDSGKTDAQKVVNKKVTIPQTGGIGTILFTIIGLSIMLGA |
| | VVIMKRRQSEEA |
| 80 | MKKKMIQSLLVASLAFGMAVSPVTPIAFAAETGTITVQDTQKGATYKAYKVFDAEIDNANVS |
| | DSNKDGASYLIPQGKEAEYKASTDFNSLFTTTTNGGRTYVTKKDTASANEIATWAKSISANT |
| | TPVSTVTESNNDGTEVINVSQYGYYYVSSTVNNGAVIMVTSVTPNATIHEKNTDATWGDGGG |
| | KTVDQKTYSVGDTVKYTITYKNAVNYHGTEKVYQYVIKDTMPSASVVDLNEGSYEVTITDGS |
| | GNITTLTQGSEKATGKYNLLEENNNFTITIPWAATNTPTGNTQNGANDDFFYKGINTITVTY |
| | TGVLKSGAKPGSADLPENTNIATINPNTSNDDPGQKVTVRDGQITIKKIDGSTKASLQGAIF |
| | VLKNATGQFLNFNDTNNVEWGTEANATEYTTGADGIITITGLKEGTYYLVEKKAPLGYNLLD |
| | NSQKVILGDGATDTTNSDNLLVNPTVENNKGTELPSTGGIGTTIFYIIGAILVIGAGIVLVA |
| | RRRLRS |
| 81 | AETGTITVQDTQKGATYKAYKVFDAEIDNANVSDSNKDGASYLIPQGKEAEYKASTDFNSLF |
| | TTTTNGGRTYVTKKDTASANEIATWAKSISANTTPVSTVTESNNDGTEVINVSQYGYYYVSS |
| | TVNNGAVIMVTSVTPNATIHEKNTDATWGDGGGKTVDQKTYSVGDTVKYTITYKNAVNYHGT |
| | EKVYQYVIKDTMPSASVVDLNEGSYEVTITDGSGNITTLTQGSEKATGKYNLLEENNNFTIT |
| | IPWAATNTPTGNTQNGANDDFFYKGINTITVTYTGVLKSGAKPGSADLPENTNIATINPNTS |
| | NDDPGQKVTVRDGQITIKKIDGSTKASLQGAIFVLKNATGQFLNFNDTNNVEWGTEANATEY |
| | TTGADGIITITGLKEGTYYLVEKKAPLGYNLLDNSQKVILGDGATDTTNSDNLLVNPTVENN |
| | KGTE |
| 82 | MKSINKFLTMLAALLLTASSLFSAATVFAAGTTTTSVTVHKLLATDGDMDKIANELETGN |
| | YAGNKVGVLPANAKEIAGVMFVWTNTNNEIIDENGQTLGVNIDPQTFKLSGAMPATAMKK |
| | LTEAEGAKFNTANLPAAKYKIYEIHSLSTYVGEDGATLTGSKAVPIEIELPLNDVVDAHV |
| | YPKNTEAKPKIDKDFKGKANPDTPRVDKDTPVNHQVGDVVEYEIVTKIPALANYATANWS |
| | DRMTEGLAFNKGTVKVTVDDVALEAGDYALTEVATGFDLKLTDAGLAKVNDQNAEKTVKI |
| | TYSATLNDKAIVEVPESNDVTFNYGNNPDHGNTPKPNKPNENGDLTLTKTWVDATGAPIP |
| | AGAEATFDLVNAQTGKVVQTVTLTTDKNTVTVNGLDKNTEYKFVERSIKGYSADYQEITT |
| | AGEIAVKNWKDENPKPLDPTEPKVVTYGKKFVKVNDKDNRLAGAEFVIANADNAGQYLAR |
| | KADKVSQEEKQLVVTTKDALDRAVAAYNALTAQQQTQQEKEKVDKAQAAYNAAVIAANNA |
| | FEWVADKDNENVVKLVSDAQGRFEITGLLAGTYYLEETKQPAGYALLTSRQKFEVTATSY |
| | SATGQGIEYTAGSGKDDATKVVNKKITIPQTGGIGTIIFAVAGAAIMGIAVYAYVKNNKD |
| | EDQLA |
| 83 | MKSINKFLTMLAALLLTASSLFSAATVFAADNVSTAPDAVTKTLTIHKLLLSEDDLKTWD |
| | TNGPKGYDGTQSSLKDLTGVVAEEIPNVYFELQKYNLTDGKEKENLKDDSKWTTVHGGLT |
| | TKDGLKIETSTLKGVYRIREDRTKTTYVGPNGQVLTGSKAVPALVTLPLVNNNGTVIDAH |
| | VFPKNSYNKPVVDKRIADTLNYNDQNGLSIGTKIPYVVNTTIPSNATFATSFWSDEMTEG |
| | LTYNEDVTITLNNVAMDQADYEVTKGNNGFNLKLTEAGLAKINGKDADQKIQITYSATLN |
| | SLAVADIPESNDITYHYGNHQDHGNTPKPTKPNNGQITVTKTWDSQPAPEGVKATVQLVN |
| | AKTGEKVGAPVELSENNWTYTWSGLDNSIEYKVEEEYNGYSAEYTVESKGKLGVKNWKDN |
| | NPAPINPEEPRVKTYGKKFVKVDQKDTRLENAQFVVKKADSNKYIAFKSTAQQAADEKAA |
| | ATAKQKLDAAVAAYTNAADKQAAQALVDQAQQEYNVAYKEAKFGYVEVAGKDEAMVLTSN |
| | TDGQFQISGLAAGTYKLEEIKAPEGFAKIDDVEFVVGAGSWNQGEFNYLKDVQKNDATKV |
| | VNKKITIPQTGGIGTIIFAVAGAAIMGIAVYAYVKNNKDEDQLA |
| 84 | MKSINKFLTILAALLLTVSSLFSAATVFAAEQKTKTLTVHKLLMTDQELDAWNSDAITTA |
| | GYDGSQNFEQFKQLQGVPQGVTEISGVAFELQSYTGPQGKEQENLTNDAVWTAVNKGVTT |
| | ETGVKFDTEVLQGTYRLVEVRKESTYVGPNGKVLTGMKAVPALITLPLVNQNGVVENAHV |
| | YPKNSEDKPTATKTFDTAAGFVDPGEKGLAIGTKVPYIVTTTIPKNSTLATAFWSDEMTE |
| | GLDYNGDVVVNYNGQPLDNSHYTLEAGHNGFILKLNEKGLEAINGKDAEATITLKYTATL |
| | NALAVADVPEANDVTFHYGNNPGHGNTPKPNKPKNGELTITKTWADAKDAPIAGVEVTFD |
| | LVNAQTGEVVKVPGHETGIVLNQTNNWTFTATGLDNNTEYKFVERTIKGYSADYQTITET |
| | GKIAVKNWKDENPEPINPEEPRVKTYGKKFVKVDQKDERLKEAQFVVKNEQGKYLALKSA |
| | AQQAVNEKAAAEAKQALDAAIAAYTNAADKNAAQAVVDAAQKTYNDNYRAARFGYVEVER |
| | KEDALVLTSNTDGQFQISGLAAGSYTLEETKAPEGFAKLGDVKFEVGAGSWNQGDFNYLK |
| | DVQKNDATKVVNKKITIPQTGGIGTIIFAVAGAVIMGIAVYAYVKNNKDEDQLA |
| 85 | MKKRQKIWRGLSVTLLILSQIPFGILVQGETQDTNQALGKVIVKKTGDNATPLGKATFVLKNDNDKS |
| | ETSHETVEGSGEATFENIKPGDYTLREETAPIGYKKTDKTWKVKVADNGATIIEGMDADKAEKRKEV |
| | LNAQYPKSAIYEDTKENYPLVNVEGSKVGEQYKALNPINGKDGRREIAEGWLSKKITGVNDLDKNKY |
| | KIELTVEGKTTVETKELNQPLDVVVLLDNSNSMNNERANNSQRALKAGEAVEKLIDKITSNKDNRVA |
| | LVTYASTIFDGTEATVSKGVADQNGKALNDSVSWDYHKTTFTATTHNYSYLNLTNDANEVNILKSRI |
| | PKEAEHINGDRTLYQFGATFTQKALMKANEILETQSSNARKKLIFHVTDGVPTMSYAINFNPYISTS |
| | YQNQFNSFLNKIPDRSGILQEDFIINGDDYQIVKGDGESFKLFSDRKVPVTGGTTQAAYRVPQNQLS |
| | VMSNEGYAINSGYIYLYWRDYNWVYPFDPKTKKVSATKQIKTHGEPTTLYFNGNIRPKGYDIFTVGI |
| | GVNGDPGATPLEAEKFMQSISSKTENYTNVDDTNKIYDELNKYFKTIVEEKHSIVDGNVTDPMGEMI |
| | EFQLKNGQSFTHDDYVLVGNDGSQLKNGVALGGPNSDGGILKDVTVTYDKTSQTIKINHLNLGSGQK |
| | VVLTYDVRLKDNYISNKFYNTNNRTTLSPKSEKEPNTIRDFPIPKIRDVREFPVLTISNQKKMGEVE |
| | FIKVNKDKHSESLLGAKFQLQIEKDFSGYKQFVPEGSDVTTKNDGKIYFKALQDGNYKLYEISSPDG |
| | YIEVKTKPVVTFTIQNGEVTNLKADPNANKNQIGYLEGNGKHLITNTPKRPPGVFPKTGGIGTIVYI |
| | LVGSTFMILTICSFRRKQL |
| 86 | MRKYQKFSKILTLSLFCLSQIPLNTNVLGESTVPENGAKGKLVVKKTDDQNKPLSKATFVLKTTAHP |
| | ESKIEKVTAELTGEATFDNLIPGDYTLSEETAPEGYKKTNQTWQVKVESNGKTTIQNSGDKNSTIGQ |
| | NQEELDKQYPPTGIYEDTKESYKLEHVKGSVPNGKSEAKAVNPYSSEGEHIREIPEGTLSKRISEVG |
| | DLAHNKYKIELTVSGKTIVKPVDKQKPLDVVFVLDNSNSMNNDGPNFQRHNKAKKAAEALGTAVKDI |
| | LGANSDNRVALVTYGSDIFDGRSVDVVKGFKEDDKYYGLQTKFTIQTENYSHKQLTNNAEEIIKRIP |
| | TEAPKAKWGSTTNGLTPEQQKEYYLSKVGETFTMKAFMEADDILSQVNRNSQKIIVHVTDGVPTRSY |
| | AINNFKLGASYESQFEQMKKNGYLNKSNFLLTDKPEDIKGNGESYFLFPLDSYQTQIISGNLQKLHY |
| | LDLNLNYPKGTIYRNGPVKEHGTPTKLYINSLKQKNYDIFNFGIDISGFRQVYNEEYKKNQDGTFQK |
| | LKEEAFKLSDGEITELMRSFSSKPEYYTPIVTSADTSNNEILSKIQQQFETILTKENSIVNGTIEDP |
| | MGDKINLQLGNGQTLQPSDYTLQGNDGSVMKDGIATGGPNNDGGILKGVKLEYIGNKLYVRGLNLGE |
| | GQKVTLTYDVKLDDSFISNKFYDTNGRTTLNPKSEDPNTLRDFPIPKIRDVREYPTITIKNEKKLGE |
| | IEFIKVDKDNNKLLLKGATFELQEFNEDYKLYLPIKNNNSKVVTGENGKISYKDLKDGKYQLIEAVS |
| | PEDYQKITNKPILTFEVVKGSIKNIIAVNKQISEYHEEGDKHLITNTHIPPKGIIPMTGGKGILSFI |
| | LIGGAMMSIAGGIYIWKRYKKSSDMSIKKD |
| 87 | MRKYQKFSKILTLSLFCLSQIPLNTNVLGESTVPENGAKGKLVVKKTDDQNKPLSKATFVLKPTSHS |
| | ESKVEKVTTEVTGEATFDNLTPGDYTLSEETAPEGYKKTTQTWQVKVESNGKTTIQNSDDKKSIIEQ |
| | RQEELDKQYPLTGAYEDTKESYNLEHVKNSIPNGKLEAKAVNPYSSEGEHIREIQEGTLSKRISEVN |
| | DLDHNKYKIELTVSGKSIIKTINKDEPLDVVFVLDNSNSMKNNGKNNKAKKAGEAVETIIKDVLGAN |
| | VENRAALVTYGSDIFDGRTVKVIKGFKEDPYYGLETSFTVQTNDYSYKKFTNIAADIIKKIPKEAPE |
| | AKWGGTSLGLTPEKKREYDLSKVGETFTMKAFMEADTLLSSIQRKSRKIIVHLTDGVPTRSYAINSF |
| | VKGSTYANQFERIKEKGYLDKNNYFITDDPEKIKGNGESYFLFPLDSYQTQIISGNLQKLHYLDLNL |
| | NYPKGTIYRNGPVREHGTPTKLYINSLKQKNYDIFNFGIDISGFRQVYNEDYKKNQDGTFQKLKEEA |
| | FELSDGEITELMNSFSSKPEYYTPIVTSADVSNNEILSKIQQQFEKILTKENSIVNGTIEDPMGDKI |
| | NLHLGNGQTLQPSDYTLQGNDGSIMKDSIATGGPNNDGGILKGVKLEYIKNKLYVRGLNLGEGQKVT |
| | LTYDVKLDDSFISNKFYDTNGRTTLNPKSEEPDTLRDFPIPKIRDVREYPTITIKNEKKLGEIEFTK |
| | VDKDNNKLLLKGATFELQEFNEDYKLYLPIKNNNSKVVTGENGKISYKDLKDGKYQLIEAVSPKDYQ |
| | KITNKPILTFEVVKGSIQNIIAVNKQISEYHEEGDKHLITNTHIPPKGIIPMTGGKGILSFILIGGA |
| | MMSIAGGIYIWKRHKKSSDASIEKD |
| 88 | MRKYQKFSKILTLSLFCLSQIPLNTNVLGESTVPENGAKGKLVVKKTDDQNKPLSKATFVLKTTAHP |
| | ESKIEKVTAELTGEATFDNLIPGDYTLSEETAPEGYKKTNQTWQVKVESNGKTTIQNSGDKNSTIGQ |
| | NQEELDKQYPPTGIYEDTKESYKLEHVKGSVPNGKSEAKAVNPYSSEGEHIREIPEGTLSKRISEVG |
| | DLAHNKYKIELTVSGKTIVKPVDKQKPLDVVFVLDNSNSMNNDGPNFQRHNKAKKAAEALGTAVKDI |
| | LGANSDNRVALVTYGSDIFDGRSVDVVKGFKEDDKYYGLQTKFTIQTENYSHKQLTNNAEEIIKRIP |
| | TEAPKAKWGSTTNGLTPEQQKEYYLSKVGETFTMKAFMEADDILSQVNRNSQKIIVHVTDGVPTRSY |
| | AINNFKLGASYESQFEQMKKNGYLNKSNFLLTDKPEDIKGNGESYFLFPLDSYQTQIISGNLQKLHY |
| | LDLNLNYPKGTFYRNGPVREHGTPTKLYINSLKQKNYDIFNFGIDISGFRQVYNEDYKKNQDGTFQK |
| | LKEEAFELSDGEITELMKSFSSKPEYYTPIVTSSDASNNEILSKIQQQFEKILTKENSIVNGTIEDP |
| | MGDKINLQLGNGQTLQPSDYTLQGNDGSIMKDSIATGGPNNDGGILKGVKLEYIKNKLYVRGLNLGE |
| | GQKVTLTYDVKLDDSFISNKFYDTNGRTTLNPKSEDPNTLRDFPIPKIRDVREYPTITIKNEKKLGE |
| | IEFTKVDKDNNKLLLKGATFELQEFNEDYKLYLPIKNNNSKVVTGENGKISYKDLKDGKYQLIEAVS |
| | PKDYQKITNKPILTFEVVKGSIQNIIAVNKQISEYHEEGDKHLITNTHIPPKGIIPMTGGKGILSFI |
| | LIGGSMMSIAGGIYIWKRYKKSSDISREKD |
| 89 | MRKYQKFSKILTLSLFCLSQIPLNTNVLGESTVPENGAKGKLVVKKTDDQNKPLSKATFVLKTTAHP |
| | ESKIEKVTAELTGEATFDNLIPGDYTLSEETAPEGYKKTNQTWQVKVESNGKTTIQNSGDKNSTIGQ |
| | NQEELDKQYPPTGIYEDTKESYKLEHVKGSVPNGKSEAKAVNPYSSEGEHIREIPEGTLSKRISEVG |
| | DLAHNKYKIELTVSGKTIVKPVDKQKPLDVVFVLDNSNSMNNDGPNFQRHNKAKKAAEALGTAVKDI |
| | LGANSDNRVALVTYGSDIFDGRSVDVVKGFKEDDKYYGLQTKFTIQTENYSHKQLTNNAEEIIKRIP |
| | TEAPKAKWGSTTNGLTPEQQKEYYLSKVGETFTMKAFMEADDILSQVNRNSQKIIVHVTDGVPTRSY |
| | AINNFKLGASYESQFEQMKKNGYLNKSNFLLTDKPDDIKGNGESYFLFPLDSYQTQIISGNLQKLHY |
| | LDLNLNYPKGTIYRNGPVKEHGTPTKLYINSLKQKNYDIFNFGIDISGFRQVYNEEYKKNQDGTFQK |
| | LKEEAFKLSDGEITELMRSFSSKPEYYTPIVTSADTSNNEILSKIQQQFETILTKENSIVNGTIEDP |
| | MGDKINLQLGNGQILQPSDYTLQGNDGSVMKDGIATGGPNNDGGILKGVKLEYIGNKLYVRGLNLGE |
| | GQKVTLTYDVKLDDSFISNKFYDTNGRTTLNPKSEDPNTLRDFPIPKIRDVREYPTITIKNEKKLGE |
| | IEFIKVDKDNNKLLLKGATFELQEFNEDYKLYLPIKNNNSKVVTGENGKISYKDLKDGKYQLIEAVS |
| | PEDYQKITNKPILTFEVVKGSIKNIIAVNKQISEYHEEGDKHLITNTHIPPKGIIPKTGGKGILSFI |
| | LIGGAMMSIAGGIYIWKRYKKSSDMSIKKD |
| 90 | MRKYQKFSKILTLSLFCLSQIPLNTNVLGESTVPENGAKGKLVVKKTDDQNKPLSKATFVLKTTAHP |
| | ESKIEKVTAELTGEATFDNLIPGDYTLSEETAPEGYKKTNQTWQVKVESNGKTTIQNSGDKNSTIGQ |
| | NHEELDKQYPPTGIYEDTKESYKLEHVKGSVPNGKSEAKAVNPYSSEGEHIREIPEGTLSKRISEVG |
| | DLAHNKYKIELTVSGKTIVKPVDKQKPLDVVFVLDNSNSMNNDGPNFQRHNKAKKAAEALGTAVKDI |
| | LGANSDNRVALVTYGSDIFDGRSVDVVKGFKEDDKYYGLQTKFTIQTENYSHKQLTNNAEEIIKRIP |
| | TEAPRAKWGSTTNGLTPEQQKQYYLSKVGETFTMKAFMEADDILSQVDRNSQKIIVHITDGVPTRSY |
| | AINNFKLGASYESQFEQMKKNGYLNKSNFLLTDKPEDIKGNGESYFLFPLDSYQTQIISGNLQKLHY |
| | LDLNLNYPKGTIYRNGPVREHGTPTKLYINSLKQKNYDIFNFGIDISAFRQVYNEDYKKNQDGTFQK |
| | LKEEAFELSDGEITELMKSFSSKPEYYTPIVTSSDASNNEILSKIQQQFEKVLTKENSIVNGTIEDP |
| | MGDKINLQLGNGQTLQPSDYTLQGNDGSIMKDSIATGGPNNDGGILKGVKLEYIKNKLYVRGLNLGE |
| | GQKVTLTYDVKLDDSFISNKFYDTNGRTTLNPKSEDPNTLRDFPIPKIRDVREYPTITIKNEKKLGE |
| | IEFTKVDKDNNKLLLKGATFELQEFNEDYKLYLPIKNNNSKVVTGENGKISYKDLKDGKYQLIEAVS |
| | PKDYQKITNKPILTFEVVKGSIQNIIAVNKQISEYHEEGDKHLITNTHIPPKGIIPMTGGKGILSFI |
| | LIGGSMMSIAGGIYIWKRYKKSSDISREKD |
| 91 | MRKYQKFSKILTLSLFCLSQIPLNTNVLGESTVPENGAKGKLVVKKTDDQNKPLSKATFVLKPTSHS |
| | ESKVEKVTTEVTGEATFDNLTPGDYTLSEETAPEGYKKTTQTWQVKVESNGKTTIQNSDDKKSIIEQ |
| | RQEELDKQYPLTGAYEDTKESYNLEHVKNSIPNGKLEAKAVNPYSSEGEHIREIQEGTLSKRISEVN |
| | DLDHNKYKIELTVSGKSIIKTINKDEPLDVVFVLDNSNSMKNNGKNNKAKKAGEAVETIIKDVLGAN |
| | VENRAALVTYGSDIFDGRTVKVIKGFKEDPYYGLETSFTVQTNDYSYKKFTNIAADIIKKIPKEAPE |
| | AKWGGTSLGLTPEKKREYDLSKVGETFTMKAFMEADTLLSSIQRKSRKIIVHLTDGVPTRSYAINSF |
| | VTGSTYANQFERIKEKGYLDKNNYFITDDPEKIKGNGESYFLFPLDSYQTQIISGNLQKLHYLDLNL |
| | NYPKGTIYRNGPVREHGTPTKLYINSLKQKNYDIFNFGIDISGFRQVYNEDYKKNQDGTFQKLKEEA |
| | FELSDGEITELMNSFSSKPEYYTPIVTSADVSNNEILSKIQQQFEKILTKENSIVNGTIEDPMGDKI |
| | NLQLGNGQTLQPSDYTLQGNDGSIMKDSIATGGPNNDGGILKGVKLEYIKNKLYVRGLNLGEGQKVT |
| | LTYDVKLDDSFISNKFYDTNGRTTLNPKSEEPDTLRDFPIPKIRDVREYPTITIKNEKKLGEIEFTK |
| | VDKDNNKLLLKGATFELQEFNEDYKLYLPIKNNNSKVVTGENGKISYKDLKDGKYQLIEAVSPKDYQ |
| | KITNKPILTFEVVKGSIQNIIAVNKQISEYHEEGDKHLITNTHIPPKGIIPMTGGKGILSFILIGGA |
| | MMSIAGGIYIWKRHKKSSDASIEKD |
| 92 | MRKYQKFSKILTLSLFCLSQIPLNTNVLGESTVPENGAKGKLVVKKTDDQNKPLSKATFVLKTTAHP |
| | ESKIEKVTAEVTGEATFDNLTPGDYTLSEETAPEGYKKTTQTWQVKVESNGKTTIQNSDDKKSIIEQ |
| | RQEELDKQYPLTGAYEDTKESYNLEHVKNSIPNGKLEAKAVNPYSSEGEHIREIQEGTLSKRISEVN |
| | DLDHNKYKIELTVSGKSIIKTINKDEPLDVVFVLDNSNSMKNNGKNNKAKKAGEAVETIIKDVLGAN |
| | VENRAALVTYGSDIFDGRTVKVIKGFKEDPYHGLETSFTVQTNDYSYKKFTNIAADIIKKIPKEAPE |
| | AKWGGTSLGLTPEKKREYDLSKVGETFTMKAFMEADTLLSSIQRKSRKIIVHLTDGVPTRSYAINSF |
| | VTGSTYANQFERIKEKGYLDKNNYFITDDPEKIKGNGESYFLFPLDSYQTQIISGNLQKLHYLDLNL |
| | NYPKGTIYRNGPVREHGTPTKLYINSLKQKNYDIFNFGIDISGFRQVYNEDYKKNQDGTFQKLKEEA |
| | FELSGGEITELMKSFSSKPEYYTPIVTSADVSNNEILSKIQQQFEKILTKENSIVNGTIEDPMGDKI |
| | NLQLGNGQTLQPSDYTLQGNDGSIMKDSIATGGPNNDGGILKGVKLEYIKNKLYVRGLNLGEGQKVT |
| | LTYDVKLDDSFISNKFYDTNGRTTLNPKSEEPDTLRDFPIPKIRDVREYPTITIKNEKKLGEIEFTK |
| | VDKDNNKLLLKGATFELQEFNEDYKLYLPIKNNNSKVVTGENGKISYKDLKDGKYQLIEAVSPKDYQ |
| | KITNKPILTFEVVKGSIQNIIAVNKQISEYHEEGDKHLITNTHIPPKGIIPMTGGKGILSFILIGGA |
| | MMSIAGGIYIWKKHKKSSDASIEKD |
| 93 | MLKKCQTFIIESLKKKKHPKEWKIIMWSLMILTTFLTTYFLILPAITVEETKTDDVGITLENKNSSQ |
| | VTSSTSSSQSSVEQSKPQTPASSVTETSSSEEAAYREEPLMFRGADYTVTVTLTKEAKIPKNADLKV |
| | TELKDNSATFKDYKKKALTEVAKQDSEIKNFKLYDITIESNGKEAEPQAPVKVEVNYDKPLEASDEN |
| | LKVVHFKDDGQTEVLKSKDTAETKNTSSDVAFKTDSFSIYAIVQEDNTEVPRLTYHFQNNDGTDYDF |
| | LTASGMQVHHQIIKDGESLGEVGIPTIKAGEHFNGWYTYDPTTGKYGDPVKFGEPITVTETKEICVR |
| | PFMSKVATVTLYDDSAGKSILERYQVPLDSSGNGTADLSSFKVSPPTSTLLFVGWSKTQNGAPLSES |
| | EIQALPVSSDISLYPVFKESYGVEFNTGDLSTGVTYIAPRRVLTGQPASTIKPNDPTRPGYTFAGWY |
| | TAASGGAAFDFNQVLTKDTTLYAHWSPAQTTYTINYWQQSATDNKNATDAQKTYEYAGQVTRSGLSL |
| | SNQTLTQQDINDKLPTGFKVNNTRTETSVMIKDDGSSVVNVYYDRKLITIKFAKYGGYSLPEYYYSY |
| | NWSSDADTYTGLYGTTLAANGYQWKTGAWGYLANVGNNQVGTYGMSYLGEFILPNDTVDSDVIKLFP |
| | KGNIVQTYRFFKQGLDGTYSLADTGGGAGADEFTFTEKYLGFNVKYYQRLYPDNYLFDQYASQTSAG |
| | VKVPISDEYYDRYGAYHKDYLNLVVWYERNSYKIKYLDPLDNTELPNFPVKDVLYEQNLSSYAPDTT |
| | TVQPKPSRPGYVWDGKWYKDQAQTQVFDFNTTMPPHDVKVYAGWQKVTYRVNIDPNGGRLSKTDDTY |
| | LDLHYGDRIPDYTDITRDYIQDPSGTYYYKYDSRDKDPDSTKDAYYTTDTSLSNVDTTTKYKYVKDA |
| | YKLVGWYYVNPDGSIRPYNFSGAVTQDINLRAIWRKAGDYHIIYSNDAVGTDGKPALDASGQQLQTS |
| | NEPTDPDSYDDGSHSALLRRPTMPDGYRFRGWWYNGKIYNPYDSIDIDAHLADANKNITIKPVIIPV |
| | GDIKLEDTSIKYNGNGGTRVENGNVVTQVETPRMELNSTTTIPENQYFTRTGYNLIGWHHDKDLADT |
| | GRVEFTAGQSIGIDNNPDATNTLYAVWQPKEYTVRVSKTVVGLDEDKTKDFLFNPSETLQQENFPLR |
| | DGQTKEFKVPYGTSISIDEQAYDEFKVSESITEKNLATGEADKTYDATGLQSLTVSGDVDISFTNTR |
| | IKQKVRLQKVNVENDNNFLAGAVFDIYESDANGNKASHPMYSGLVTNDKGLLLVDANNYLSLPVGKY |
| | YLTETKAPPGYLLPKNDISVLVISTGVTFEQNGNNATPIKENLVDGSTVYTFKITNSKGTELPSTGG |
| | IGTHIYILVGLALALPSGLILYYRKKI |
| 94 | MKKVRKIFQKAVAGLCCISQLTAFSSIVALAETPETSPAIGKVVIKETGEGGALLGDAVFELKNNTD |
| | GTTVSQRTEAQTGEAIFSNIKPGTYTLTEAQPPVGYKPSTKQWTVEVEKNGRTTVQGEQVENREEAL |
| | SDQYPQTGTYPDVQTPYQIIKVDGSEKNGQHKALNPNPYERVIPEGTLSKRIYQVNNLDDNQYGIEL |
| | TVSGKTVYEQKDKSVPLDVVILLDNSNSMSNIRNKNARRAERAGEATRSLIDKITSDPENRVALVTY |
| | ASTIFDGTEFTVEKGVADKNGKRLNDSLFWNYDQTSFTTNTKDYSYLKLTNDKNDIVELKNKVPTEA |
| | EDHDGNRLMYQFGATFTQKALMKADEILTQQARQNSQKVIFHITDGVPTMSYPINFNHATFAPSYQN |
| | QLNAFFSKSPNKDGILLSDFITQATSGEHTIVRGDGQSYQMFTDKTVYEKGAPAAFPVKPEKYSEMK |
| | AAGYAVIGDPINGGYIWLNWRESILAYPFNSNTAKITNHGDPTRWYYNGNIAPDGYDVFTVGIGING |
| | DPGTDEATATSFMQSISSKPENYTNVTDTTKILEQLNRYFHTIVTEKKSIENGTITDPMGELIDLQL |
| | GTDGRFDPADYTLTANDGSRLENGQAVGGPQNDGGLLKNAKVLYDTTEKRIRVTGLYLGTDEKVTLT |
| | YNVRLNDEFVSNKFYDTNGRTTLHPKEVEQNTVRDFPIPKIRDVRKYPEITISKEKKLGDIEFIKVN |
| | KNDKKPLRDAVFSLQKQHPDYPDIYGAIDQNGTYQNVRTGEDGKLTFKNLSDGKYRLFENSEPAGYK |
| | PVQNKPIVAFQIVNGEVRDVTSIVPQDIPAGYEFTNDKHYITNEPIPPKREYPRTGGIGMLPFYLIG |
| | CMMMGGVLLYTRKHP |
| 95 | MKQTLKLMFSFLLMLGTMFGISQTVLAQETHQLTIVHLEARDIDRPNPQLEIAPKEGTPIEGVLYQL |
| | YQLKSTEDGDLLAHWNSLTITELKKQAQQVFEATTNQQGKATFNQLPDGIYYGLAVKAGEKNRNVSA |
| | FLVDLSEDKVIYPKIIWSTGELDLLKVGVDGDTKKPLAGVVFELYEKNGRTPIRVKNGVHSQDIDAA |
| | KHLETDSSGHIRISGLIHGDYVLKEIETQSGYQIGQAETAVTIEKSKTVTVTIENKKVPTPKVPSRG |
| | GLIPKTGEQQAMALVIIGGILIALALRLLSKHRKHQNKD |
| 96 | MKKIRKSLGLLLCCFLGLVQLAFFSVASVNADTPNQLTITQIGLQPNTTEEGISYRLWTVTDNLKVD |
| | LLSQMTDSELNQKYKSILTSPTDTNGQTKIALPNGSYFGRAYKADQSVSTIVPFYIELPDDKLSNQL |
| | QINPKRKVETGRLKLIKYTKEGKIKKRLSGVIFVLYDNQNQPVRFKNGRFTTDQDGITSLVTDDKGE |
| | IEVEGLLPGKYIFREAKALTGYRISMKDAVVAVVANKTQEVEVENEKETPPPTNPKPSQPLFPQSFL |
| | PKTGMIIGGGLTILGCIILGILFIFLRKTKNSKSERNDTV |
| 97 | MTMQKMQKMISRIFFVMALCFSLVWGAHAVQAQEDHTLVLQLENYQEVVSQLPSRDGHRLQVWKLDD |
| | SYSYDDRVQIVRDLHSWDENKLSSFKKTSFEMTFLENQIEVSHIPNGLYYVRSIIQTDAVSYPAEFL |
| | FEMTDQTVEPLVIVAKKTDTMTTKVKLIKVDQDHNRLEGVGFKLVSVARDGSEKEVPLIGEYRYSSS |
| | GQVGRTLYTDKNGEIFVTNLPLGNYRFKEVEPLAGYAVTTLDTDVQLVDHQLVTITVVNQKLPRGNV |
| | DFMKVDGRTNTSLQGAMFKVMKEESGHYTPVLQNGKEVVVTSGKDGRFRVEGLEYGTYYLWELQAPT |
| | GYVQLTSPVSFTIGKDTRKELVTVVKNNKRPRIDVPDTGEETLYILMLVAILLFGSGYYLTKKPNN |
| 98 | HQSRAIMDYQDRVTHMDENDYKKIINRAKEYNKQFKTSGMKWHMTSQERLDYNSQLAIDKTGNMGYI |
| | SIPKINIKLPLYHGTSEKVLQTSIGHLEGSSLPIGGDSTHSILSGHRGLPSSRLFSDLDKLKVGDHW |
| | TVSILNETYTYQVDQIRTVKPDDLRDLQIVKGKDYQTLVTCTPYGVNTHRLLVRGHRVPNDNGNALV |
| | VAEAIQIEPIYIAPFIAIFLTLILLLISLEVTRRARQRKKILKQAMRKEENNDL |
| 99 | NSQLAIDKTGNMGYISIPKINIKLPLYHGTSEKVLQTSIGHLEGSSLPIGGDSTHSILSGHRGLPSS |
| | RLFSDLDKLKVGDHWTVSILNETYTYQVDQIRTVKPDDLRDLQIVKGKDYQTLVTCTPYGVNTHRLL |
| | VRGHRVPNDNGNALVVAEAIQIEPIYIAPFIAIFLTLILLLISLEVTRRARQRKKILKQAMRKEENN |
| | DL |
| 100 | QSRAIMDYQDRVTHMDENDYKKIINRAKEYNKQFKTSGMKWHMTSQERLDYNSQLAIDKTGNMGYIS |
| | IPKINIKLPLYHGTSEKVLQTSIGHLEGSSLPIGGDSTHSILSGHRGLPSSRLFSDLDKLKVGDHWT |
| | VSILNETYTYQVDQIRTVKPDDLRDLQIVKGKDYQTLVTCTPYGVNTHRLLVRGHRVPNDNGNALVV |
| | AEAIQIEPIYIAPFIAIFLTLILLLISLEVTRRARQRKKILKQAMRKEENNDL |
| 101 | QSRAIMDYQDRVTHMDENDYKKIINRAKEYNKQFKTSGMKWHMTSQERLDYNSQLAIDKTGNMGYIS |
| | IPKINIKLPLYHGTSEKVLQTSIGHLEGSSLPIGGDSTHSILSGHRGLPSSRLFSDLDKLKVGDHWT |
| | VSILNETYTYQVDQIRTVKPDDLRDLQIVKGKDYQTLVTCTPYGVNTHRLLVRGHRVPNDNGN |
| 102 | QSRAIMDYQDRVTHMDENDYKKIINRAKEYNKQFKTSGMKAHMTSQERLDYNSQLAIDKTGNMGYIS |
| | IPKINIKLPLYHGTSEKVLQTSIGHLEGSSLPIGGDSTHSILSGHRGLPSSRLFSDLDKLKVGDHWT |
| | VSILNETYTYQVDQIRTVKPDDLRDLQIVKGKDYQTLVTCTPYGVNTHRLLVRGHRVPNDNGNALVV |
| | AEAIQIEPIYIAPFIAIFLTLILLLISLEVTRRARQRKKILKQAMRKEENNDL |
| 103 | QSRAIMDYQDRVTHMDENDYKKIINRAKEYNKQFKTSGMKWHMTSQERLDYNSQLAIDKTGNMGYIS |
| | IPKINIKLPLYHGTSEKVLQTSIGHLEGSSLPIGGDSTHSILSGHRGLPSSRLFSDLDKLKVGDHWT |
| | VSILNETYTYQVDQIRTVKPDDLRDLQIVKGKDYQTLVTATPYGVNTHRLLVRGHRVPNDNGNALVV |
| | AEAIQIEPIYIAPFIAIFLTLILLLISLEVTRRARQRKKILKQAMRKEENNDL |
| 104 | ATGGCTTATCCTTCACTTGCTAATTATTGGAATTCATTTCACCAATCTCGAGCGATTATGGATTACC |
| | AAGACCGCGTAACGCATATGGATGAAAACGATTATAAAAAAATTATTAACCGAGCCAAAGAATATAA |
| | TAAGCAATTTAAAACTTCAGGAATGAAGTGGCACATGACTAGCCAAGAGCGTTTGGATTATAATTCA |
| | CAACTGGCTATCGATAAAACGGGTAATATGGGTTATATTTCAATTCCAAAGATAAACATAAAATTAC |
| | CACTTTATCATGGTACAAGTGAAAAAGTGCTTCAAACTTCTATTGGTCATTTAGAAGGAAGTAGTCT |
| | TCCAATTGGAGGAGACTCAACTCATTCTATTTTATCAGGACATAGAGGTTTACCCTCTTCAAGGCTT |
| | TTTTCTGATTTGGATAAGTTAAAAGTTGGAGACCACTGGACAGTCAGTATCTTAAATGAAACATATA |
| | CTTATCAAGTGGATCAAATCAGAACAGTTAAACCGGATGATTTGAGGGATTTACAAATTGTTAAAGG |
| | TAAAGACTACCAAACTTTGGTGACGTGTACACCATATGGCGTTAATACCCATCGGTTACTAGTGAGA |
| | GGACATCGTGTACCAAACGATAATGGTAACGCTTTGGTAGTAGCAGAGGCAATACAAATAGAGCCTA |
| | TTTATATCGCACCATTTATCGCTATTTTCCTTACTTTGATTTTACTTTTAATCTCTTTAGAAGTAAC |
| | TAGGAGAGCACGTCAACGTAAGAAAATTTTAAAACAAGCAATGAGAAAGGAAGAGAACAATGATTTA |
| | TAA |
| 105 | MIRRYSANFLAILGIILVSSGIYWGWYNINQAHQADLTSQHIVKVLDKSITHQVKGSENGELPVKKL |
| | DKTDYLGTLDIPNLKLHLPVAANYSFEQLSKTPTRYYGSYLTNNMVICAHNFPYHFDALKNVDMGTD |
| | VYFTTTTGQIYHYKISNREIIEPTAIEKVYKTATSDNDWDLSLFTCTKAGVARVLVRCQLIDVKN |
| 106 | MIRRYSANFLAILGIILVSSGIYWGWYNINQAHQADLTSQHIVKVLDKSITHQVKGSENGELPVKKL |
| | DKTDYLGTLDIPNLKLHLPVAANYSFEQLSKTPTRYYGSYLTNNMVIAAHNFPYHFDALKNVDMGTD |
| | VYFTTTTGQIYHYKISNREIIEPTAIEKVYKTATSDNDWDLSLFTATKAGVARVLVRAQLIDVKN |
| 107 | GTGATTAGAAGATATTCAGCAAATTTTTTAGCTATACTCGGAATTATTCTGGTAAGTTCTGGAATCT |
| | ATTGGGGTTGGTATAATATTAATCAGGCGCATCAAGCTGATTTAACTTCTCAGCATATTGTCAAGGT |
| | GCTTGATAAATCTATTACGCATCAAGTAAAGGGTTCAGAAAATGGAGAATTACCTGTAAAAAAGTTG |
| | GATAAAACAGATTACTTGGGAACTCTGGATATTCCGAACTTAAAACTGCATTTACCGGTAGCTGCTA |
| | ATTATAGTTTTGAACAACTGTCTAAGACGCCTACAAGGTATTATGGTTCTTATTTAACTAATAACAT |
| | GGTGATTTGTGCGCATAATTTTCCTTATCATTTTGATGCTTTAAAAAATGTAGATATGGGAACGGAT |
| | GTTTATTTTACAACTACAACAGGGCAAATCTATCACTACAAAATCAGTAATAGAGAAATTATTGAAC |
| | CAACAGCGATTGAAAAAGTTTATAAAACTGCCACATCAGACAATGATTGGGACTTAAGCTTGTTTAC |
| | TTGTACAAAGGCAGGAGTAGCTAGAGTATTAGTGCGCTGTCAATTAATTGATGTTAAAAATTAA |
| 108 | QAHQADLTSQHIVKVLDKSITHQVKGSENGELPVKKLDKTDYLGTLDIPNLKLHLPVAANYSFEQLS |
| | KTPTRYYGSYLTNNMVICAHNFPYHFDALKNVDMGTDVYFTTTTGQIYHYKISNREIIEPTAIEKVY |
| | KTATSDNDWDLSLFTCTKAGVARVLVRCQLIDVKN |
| 109 | LAILGIILVSSGIYWGWYNINQAHQADLTSQHIVKVLDKSITHQVKGSENGELPVKKLDKTDYLGTL |
| | DIPNLKLHLPVAANYSFEQLSKTPTRYYGSYLTNNMVICAHNFPYHFDALKNVDMGTDVYFTTTTGQ |
| | IYHYKISNREIIEPTAIEKVYKTATSDNDWDLSLFTCTKAGVARVLVRCQLIDVKN |
| 110 | MKKKMIQSLLVASLAFGMAVSPVTPIAFAAETGTITVQDTQKGATYKAYKVFDAEIDNANVSDSNKD |
| | GASYLIPQGKEAEYKASTDFNSLFTTTTNGGRTYVTKKDTASANEIATWAKSISANTTPVSTVTESN |
| | NDGTEVINVSQYGYYYVSSTVNNGAVIMVTSVTPNATIHEKNTDATWGDGGGKTVDQKTYSVGDTVK |
| | YTITYKNAVNYHGTEKVYQYVIKDTMPSASVVDLNEGSYEVTITDGSGNITTLTQGSEKATGKYNLL |
| | EENNNFTITIPWAATNTPTGNTQNGANDDFFYKGINTITVTYTGVLKSGAKPGSADLPENTNIATIN |
| | PNTSNDDPGQKVTVRDGQITIKKIDGSTKASLQGAIFVLKNATGQFLNFNDTNNVEWGTEANATEYT |
| | TGADGIITITGLKEGTYYLVEKKAPLGYNLLDNSQKVILGDGATDTTNSDNLLVNPTVENNKGTELP |
| | STGGIGTTIFYIIGAILVIGAGIVLVARRRLRS |
| 111 | AETGTITVQDTQKGATYKAYKVFDAEIDNANVSDSNKDGASYLIPQGKEAEYKASTDFNSL |
| | FTTTTNGGRTYVTKKDTASANEIATWAKSISANTTPVSTVTESNNDGTEVINVSQYGYYYV |
| | SSTVNNGAVIMVTSVTPNATIHEKNTDATWGDGGGKTVDQKTYSVGDTVKYTITYKNAVNY |
| | HGTEKVYQYVIKDTMPSASVVDLNEGSYEVTITDGSGNITTLTQGSEKATGKYNLLEENNN |
| | FTITIPWAATNTPTGNTQNGANDDFFYKGINTITVTYTGVLKSGAKPGSADLPENTNIATI |
| | NPNTSNDDPGQKVTVRDGQITIKKIDGSTKASLQGAIFVLKNATGQFLNFNDTNNVEWGTE |
| | ANATEYTTGADGIITITGLKEGTYYLVEKKAPLGYNLLDNSQKVILGDGATDTTNSDNLLV |
| | NPTVENNKGTE |
| 112 | AETGTITVQDTKKGATYKAYKVFDAEIDNANVSDSNKDGASYLIPQGKEAEYKASTDFNSL |
| | FTTTTNGGRTYVTKKDTASANEIATWAKSISANTTPVSTVTESNNDGTEVINVSQYGYYYV |
| | SSTVNNGAVIMVTSVTPNATIHEKNTDATWGDGGGKTVDQKTYSVGDTVKYTITYKNAVNY |
| | HGTEKVYQYVIKDTMPSASVVDLNEGSYEVTITDGSGNITTLTQGSEKATGKYNLLEENNN |
| | FTITIPWAATNTPTGNTQNGANDDFFYKGINTITVTYTGVLKSGAKPGSADLPENTNIATI |
| | NPNTSNDDPGQKVTVRDGQITIKKIDGSTKASLQGAIFVLKNATGQFLNFNDTNNVEWGTE |
| | ANATEYTTGADGIITITGLKEGTYYLVEKKAPLGYNLLDNSQKVILGDGATDTTNSDNLLV |
| | NPTVENNKGTE |
| 113 | attccacaaacaggtggtattggtacaTAACGCGACTTAATTAAACGG |
| 114 | TGTACCAATACCACCTGTTTGTGGAATCTTGTACAGCTCGTCCATGCC |
| 115 | CTTTAAGAAGGAGATATACATACCCATGGGATCTGATAAAATTCATCATCATCATCATCAC |
| | GAAAACCTGTACTTCCAGGGCatggtgagcaagggcgaggagctgttcaccggggtggtgc |
| | ccatcctggtcgagctggacggcgacgtaaacggccacaagttcagcgtgtccggcgaggg |
| | cgagggcgatgccacctacggcaagctgaccctgaagttcatctgcaccaccggcaagctg |
| | cccgtgccctggcccaccctcgtgaccaccctgacctacggcgtgcagtgcttcagccgct |
| | accccgaccacatgaagcagcacgacttcttcaagtccgccatgcccgaaggctacgtcca |
| | ggagcgcaccatcttcttcaaggacgacggcaactacaagacccgcgccgaggtgaagttc |
| | gagggcgacaccctggtgaaccgcatcgagctgaagggcatcgacttcaaggaggacggca |
| | acatcctggggcacaagctggagtacaactacaacagccacaacgtctatatcatggccga |
| | caagcagaagaacggcatcaaggtgaacttcaagatccgccacaacatcgaggacggcagc |
| | gtgcagctcgccgaccactaccagcagaacacccccatcggcgacggccccgtgctgctgc |
| | ccgacaaccactacctgagcacccagtccgccctgagcaaagaccccaacgagaagcgcga |
| | tcacatggtcctgctggagttcgtgaccgccgccgggatcactctcggcatggacgagctg |
| | tacaagTAACGCGACTTAATTAAACGGTCTCCAGCTTGGCTGTTTTGGCGGATGAGAGAAG |
| | ATTTTCAGCCTGATACAGATTAAATC |
| 116 | MGSDKIHHHHHHENLYFQGMVSKGEELFTGVVPILVELDGDVNGHKFSVSGEGEGDATYGK |
| | LTLKFICTTGKLPVPWPTLVTTLTYGVQCFSRYPDHMKQHDFFKSAMPEGYVQERTIFFKD |
| | DGNYKTRAEVKFEGDTLVNRIELKGIDFKEDGNILGHKLEYNYNSHNVYIMADKQKNGIKV |
| | NFKIRHNIEDGSVQLADHYQQNTPIGDGPVLLPDNHYLSTQSALSKDPNEKRDHMVLLEFV |
| | TAAGITLGMDELYK |
| 117 | CTTTAAGAAGGAGATATACATACCCATGGGATCTGATAAAATTCATCATCATCATCATCAC |
| | GAAAACCTGTACTTCCAGGGCatggtgagcaagggcgaggagctgttcaccggggtggtgc |
| | ccatcctggtcgagctggacggcgacgtaaacggccacaagttcagcgtgtccggcgaggg |
| | cgagggcgatgccacctacggcaagctgaccctgaagttcatctgcaccaccggcaagctg |
| | cccgtgccctggcccaccctcgtgaccaccctgacctacggcgtgcagtgcttcagccgct |
| | accccgaccacatgaagcagcacgacttcttcaagtccgccatgcccgaaggctacgtcca |
| | ggagcgcaccatcttcttcaaggacgacggcaactacaagacccgcgccgaggtgaagttc |
| | gagggcgacaccctggtgaaccgcatcgagctgaagggcatcgacttcaaggaggacggca |
| | acatcctggggcacaagctggagtacaactacaacagccacaacgtctatatcatggccga |
| | caagcagaagaacggcatcaaggtgaacttcaagatccgccacaacatcgaggacggcagc |
| | gtgcagctcgccgaccactaccagcagaacacccccatcggcgacggccccgtgctgctgc |
| | ccgacaaccactacctgagcacccagtccgccctgagcaaagaccccaacgagaagcgcga |
| | tcacatggtcctgctggagttcgtgaccgccgccgggatcactctcggcatggacgagctg |
| | tacaagattccacaaacaggtggtattggtacaTAACGCGACTTAATTAAACGGTCTCCAG |
| | CTTGGCTGTTTTGGCGGATGAGAGAAGATTTTCAGCCTGATACAGATTAAATC |
| 118 | MGSDKIHHHHHHENLYFQGMVSKGEELFTGVVPILVELDGDVNGHKFSVSGEGEGDATYGK |
| | LTLKFICTTGKLPVPWPTLVTTLTYGVQCFSRYPDHMKQHDFFKSAMPEGYVQERTIFFKD |
| | DGNYKTRAEVKFEGDTLVNRIELKGIDFKEDGNILGHKLEYNYNSHNVYIMADKQKNGIKV |
| | NFKIRHNIEDGSVQLADHYQQNTPIGDGPVLLPDNHYLSTQSALSKDPNEKRDHMVLLEFV |
| | TAAGITLGMDELYKIPQTGGIGT |

### REFERENCES

[1] Rosini et al, Molecular Microbiology, 2006, 61(1): 126-141
[2] Margarit et al, Journal of Infectious Diseases, 2009, 199: 108-115
[3] Cozzi et al, The FASEBJ, 2011, 25: 1874-1886
[4] Manzano et al, 2008, Structure, 16: 1838-1848
[5] Neiers et al, 2009, J. Mol. Biol. 393, 704-716
[6] Cozzi et al, 2012, FASEBJ, 26:1-11
[7] Telford et al, 2006, Nature Reviews Microbiology 4: 509-519
[8] Gennaro (2000) Remington: The Science and Practice of Pharmacy. 20th edition, ISBN: 0683306472.
[9] Vaccine Design (1995) eds. Powell & Newman. ISBN: 030644867X. Plenum.
[10] WO90/14837.
[11] WO90/14837.
[12] Podda & Del Giudice (2003) Expert Rev Vaccines 2:197-203.
[13] Podda (2001) Vaccine 19: 2673-2680.
[14] Vaccine Design: The Subunit and Adjuvant Approach (eds. Powell & Newman)
Plenum Press 1995 (ISBN 0-306-44867-X).
[15] Vaccine Adjuvants: Preparation Methods and Research Protocols (Volume 42 of Methods in Molecular Medicine series). ISBN: 1-59259-083-7. Ed. O'Hagan.
[16] U.S. Pat. No. 5,057,540.
[17] Niikura et al. (2002) Virology 293:273-280.
[18] Lenz et al. (2001) J Immunol 166:5346-5355.
[19] Pinto et al. (2003) J Infect Dis 188:327-338.
[20] Gerber et al. (2001) J Virol 75:4752-4760.
[21] WO03/024480.
[22] WO03/024481.
[23] Gluck et al. (2002) Vaccine 20:B10-B16.
[24] Meraldi et al. (2003) Vaccine 21:2485-2491.
[25] Pajak et al. (2003) Vaccine 21:836-842.
[26] Krieg (2003) Nature Medicine 9:831-835.
[27] McCluskie et al. (2002) FEMS Immunology and Medical Microbiology 32:179-185.
[28] WO98/40100.
[29] U.S. Pat. No. 6,207,646.
[30] U.S. Pat. No. 6,239,116.
[31] U.S. Pat. No. 6,429,199.
[32] Schellack et al. (2006) Vaccine 24:5461-72.
[33] Johnson et al. (1999) Bioorg Med Chem Lett 9:2273-2278.
[34] Evans et al. (2003) Expert Rev Vaccines 2:219-229.
[35] Beignon et al. (2002) Infect Immun 70:3012-3019.
[36] Pizza et al. (2001) Vaccine 19:2534-2541.
[37] Pizza et al. (2000) Int J Med Microbiol 290:455-461.
[38] Scharton-Kersten et al. (2000) Infect Immun 68:5306-5313.
[39] Ryan et al. (1999) Infect Immun 67:6270-6280.
[40] Partidos et al. (1999) Immunol Lett 67:209-216.
[41] Peppoloni et al. (2003) Expert Rev Vaccines 2:285-293.
[42] Pine et al. (2002) J Control Release 85:263-270.
[43] WO99/40936.
[44] WO99/44636.
[45] Singh et al] (2001) J Cont Release 70:267-276.
[46] WO99/27960.
[47] U.S. Pat. No. 6,090,406.
[48] U.S. Pat. No. 5,916,588.
[49] EP-A-0626169.
[50] WO99/52549.
[51] Andrianov et al. (1998) Biomaterials 19:109-115.
[52] Payne et al. (1998) Adv Drug Delivery Review 31:185-196.
[53] Stanley (2002) Clin Exp Dermatol 27:571-577.
[54] Jones (2003) Curr Opin Investig Drugs 4:214-218.
[55] WO99/11241.
[56] WO94/00153.
[57] WO98/57659.
[58] European patent applications 0835318, 0735898 and 0761231.
[59] Ogunniyi et al. (2001) Infect Immun 69:5997-6003.
[60] WO2006/110603.
[61] Watson (2000) Pediatr Infect Dis J 19:331-332.
[62] Rubin (2000) Pediatr Clin North Am 47:269-285, v.
[63] Jedrzejas (2001) Microbiol Mol Biol Rev 65:187-207.
[64] Bell (2000) Pediatr Infect Dis J 19:1187-1188.
[65] Iwarson (1995) APMIS 103:321-326.
[66] Gerlich et al. (1990) Vaccine 8 Suppl:S63-68 & 79-80.
[67] Vaccines (1988) eds. Plotkin & Mortimer. ISBN 0-7216-1946-0.
[68] Del Guidice et al. (1998) Molecular Aspects of Medicine 19:1-70.
[69] Gustafsson et al. (1996) N. Engl. J. Med. 334:349-355.
[70] Rappuoli et al. (1991) TIBTECH 9:232-238.
[71] Costantino et al. (1999) Vaccine 17:1251-1263.
[72] Sutter et al. (2000) Pediatr Clin North Am 47:287-308.
[73] Zimmerman & Spann (1999) Am Fam Physician 59:113-118, 125-126.
[74] McMichael (2000) Vaccine 19 Suppl 1:S101-107.
[75] Schuchat (1999) Lancet 353(9146):51-6.
[76] WO02/34771.
[77] Dale (1999) Infect Dis Clin North Am 13:227-43, viii.
[78] Ferretti et al. (2001) PNAS USA 98: 4658-4663.
[79] Kuroda et al. (2001) Lancet 357(9264):1225-1240; see also pages 1218-1219.
[80] EP-A-0372501
[81] EP-A-0378881
[82] EP-A-0427347
[83] WO93/17712
[84] WO94/03208
[85] WO98/58668
[86] EP-A-0471177
[87] WO00/56360
[88] WO91/01146
[89] WO00/61761
[90] WO01/72337
[91] Research Disclosure, 453077 (Jan 2002)
[92] Gennaro (2000) Remington: The Science and Practice of Pharmacy. 20th edition, ISBN: 0683306472.
[93] Methods In Enzymology (S. Colowick and N. Kaplan, eds., Academic Press, Inc.)
[94] Handbook of Experimental Immunology, Vols. I-IV (D. M. Weir and C. C. Blackwell, eds, 1986, Blackwell Scientific Publications)
[95] Sambrook et al. (2001) Molecular Cloning: A Laboratory Manual, 3rd edition (Cold Spring Harbor Laboratory Press).
[96] Handbook of Surface and Colloidal Chemistry (Birdi, K. S. ed., CRC Press, 1997)
[97] Ausubel et al. (eds) (2002) Short protocols in molecular biology, 5th edition (Current Protocols).
[98] Molecular Biology Techniques: An Intensive Laboratory Course, (Ream et al., eds., 1998, Academic Press)
[99] PCR (Introduction to Biotechniques Series), 2nd ed. (Newton & Graham eds., 1997, Springer Verlag)
[100] Current Protocols in Molecular Biology (F. M. Ausubel et al., eds., 1987) Supplement 30
[101] Smith & Waterman (1981) Adv. Appl. Math. 2: 482-489.
[102] Sendi P et al, (2008) Infection. 36(2):100-11
[103] Dramsi S et al, (2006), Mol Microbiol. 60(6):1401-13.
[104] Nobbs A H, et at (2008) Infect Immun.; 76(8):3550-60.
[105] Persson K. (2011) Acta Crystallogr D Biol Crystallogr. 67(Pt 3):212-7.
[106] Lu G, et al, (2011) Proteins. 79(9):2764-9.
[107] Nuccitelli A, et at (2011) Proc Natl Acad Sci USA. 21; 108(25):10278-83.
[108] Maione D, et al (2005) Science 309(5731):148-50

1. A method of ligating at least two moieties comprising contacting the at least two moieties with a pilus-related sortase C enzyme in vitro under conditions suitable for a sortase mediated transpeptidation reaction to occur, wherein the pilus-related sortase C enzyme comprises an exposed active site.

2. The method of claim 1, wherein the pilus-related sortase C enzyme comprises an amino acid sequence having at least 60% identity to or at least 50 consecutive amino acids of a sortase C polypeptide from Streptococcus.

3. The method of claim 2 wherein the Streptococcus is selected from the group consisting of Streptococcus agalactiae (GBS), Streptococcus pneumonia (pneumococcus) and Streptococcus pyogenes (GAS).

4. The method of claim 1, wherein the pilus-related sortase C enzyme is a sortase C1 enzyme (srtC1), sortase C2 enzyme (SrtC2), a sortase C3 enzyme (SrtC3), or combination thereof.

5. The method of claim 1, wherein the pilus-related sortase C enzyme comprises a deletion of part or all of the lid region of the sortase C enzyme.

6. The method of claim 1, wherein the pilus-related sortase C enzyme comprises a mutation at positions 84, 85 and/or 86 of the amino acid sequence of the GBS sortase C1 enzyme of PI-2a (SEQ ID NO:3), or at corresponding positions in the amino acid sequence of another pilus-related sortase C enzyme, wherein the mutation is a deletion, substitution, or combination thereof.

7. (canceled)

8. The method of claim 1, wherein the pilus-related sortase C enzyme comprises or consists of an amino acid sequence selected from the group consisting of SEQ ID NO: 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70 and 71.

9. The method of claim 1, wherein the at least two moieties comprise an LPxTG motif, a pilin motif, an E-Box motif, or combination thereof.

10. The method of claim 1, wherein the at least two moieties correspond to polypeptides of Gram-positive bacteria.

11. (canceled)

12. The method of claim 10, wherein the at least two moieties are polypeptides having 50% or more identity to or a fragment with at least 20 consecutive amino acids of Streptococcal backbone proteins and/or ancillary proteins.

13. (canceled)

14. The method of claim 12, wherein the at least two moieties comprise or consist of an amino acid sequence: (a) having 50% or more identity to a polypeptide having the amino acid sequence of any one of SEQ ID NOs: 72, 73, 74, 75, 76, 77, 78, 79, 80, 81, 82, 83, 84, 85, 86, 87, 88, 89, 90, 91, 92, 93, 94, 95, 96, or 97; or (b) that is a fragment of at least ‘n’ consecutive amino acids of one of these sequences wherein ‘n’ is 20 or more.

15. An artificial pilus obtained or obtainable by the method of claim 1.

16. An artificial pilus which comprises at least two variants of backbone protein GBS59 and wherein the at least two variants are selected from the group consisting of Group B Streptococcus strains 2603, H36B, 515, CJB111, CJB110 and DK21.

17-18. (canceled)

19. A method of treating or preventing Streptococcal infection in a patient in need thereof comprising administering to the patient the artificial pilus of claim 15 or 16 in an amount effective to treat or prevent the Streptococcal infection.

20. The method of claim 1, wherein the at least two moieties comprise a first moiety comprising the amino acid motif LPXTG, wherein X is any amino acid, and a second moiety comprising at least one amino acid.

21-25. (canceled)

26. The method of claim 20, wherein either the first moiety or the second moiety comprises a detectable label.

27. (canceled)

28. The method according to claim 20, wherein either the first moiety or the second moiety is:
i) a polypeptide and the other moiety is a protein or glycoprotein on the surface of a cell;
ii) a polypeptide and the other moiety comprises amino acids conjugated to a solid support; or,
iii) a polypeptide and the other moiety comprises at least one amino acid conjugated to a polynucleotide.

29-30. (canceled)

31. The method of claim 20, wherein the first moiety and the second moiety are the N-terminus and C-terminus of a polypeptide chain, and ligation results in the formation of a circular polypeptide.

32. (canceled)

33. A kit comprising a PI-2b sortase C1 or a PI-2b sortase C2 enzyme from Streptococcus agalactiae and a moiety comprising the amino acid motif LPXTG, wherein X is any amino acid.

34. A conjugate obtained or obtainable by the method of claim 20.
